(19) United States
(12) Patent Application Publication
(10) Pub. No.: US 2026/0364375 A1
(43) Pub. Date: Nov. 19, 2026

(54) SYSTEMS, METHODS, AND APPARATUS FOR ADAPTIVE MULTI-SITE NEUROSTIMULATION USING CLOSED-LOOP PHYSIOLOGICAL FEEDBACK AND FRACTIONAL-ORDER CONTROL

(71) Applicant: Quietmind Associates LLC, Linwood, NJ (US)

(72) Inventor: Marvin Berman, Egg Harbor, NJ (US)

(73) Assignee: Quietmind Associates LLC, Linwood, NJ (US)

(21) Appl. No.: 19/675,389

(22) Filed: May 12, 2026

Related U.S. Application Data
(60) Provisional application No. 63/806,110, filed on May 15, 2025.

(51) Int. Cl. A61N 5/06; A61M 21/02

(52) U.S. Cl. CPC A61N 5/0622; A61M 21/02; A61N 5/0603; A61N 5/0618; A61M 2205/3303; A61M 2205/3368; A61M 2205/52; A61M 2209/088; A61M 2210/0618; A61M 2210/0693; A61M 2210/1021; A61M 2230/005; A61M 2230/04; A61M 2230/10; A61M 2230/20; A61N 2005/0607; A61N 2005/0628; A61N 2005/0632; A61N 2005/0647; A61N 2005/0652; A61N 2005/0658

(57) ABSTRACT

A neurostimulation system includes a cranial photobiomodulation module, an intranasal stimulation module, an abdominal photobiomodulation module, a sensor subsystem, and a controller. The sensor subsystem acquires physiological signals during a stimulation session, including electroencephalographic, heart rate variability, and peripheral oxygen saturation signals. The controller extracts physiological features from the signals and maintains a fractional-order dynamic model representing memory-dependent physiological response to coordinated stimulation. The controller generates a predicted physiological response for candidate stimulation parameters, compares the predicted physiological response with a measured physiological response, and selects updated stimulation parameters for the cranial, intranasal, and abdominal modules based on an error signal and a metabolic-substrate input pathway associated with short-chain fatty acid availability. The controller iteratively updates stimulation parameters during the stimulation session to converge the measured physiological response toward a target physiological state.

### CROSS REFERENCE TO RELATED APPLICATIONS

This application claims the benefit of U.S. Provisional Application No. 63/806,110, filed May 15, 2025, the entire disclosure of which is incorporated herein by reference.

### FIELD OF INVENTION

The present invention relates generally to the field of neurostimulation and cognitive modulation technologies. More particularly, the invention pertains to wearable systems, apparatuses, and methods for delivering personalized, adaptive neurostimulation therapies using energy emitters and sensor feedback. The invention incorporates artificial intelligence (AI) to dynamically configure, and control stimulation parameters based on physiological, neurological, and behavioral inputs, with applications in cognitive enhancement, emotional regulation, stress relief, sleep modulation, rehabilitation, and related domains.

The present invention further relates to closed-loop, adaptive neurostimulation and photobiomodulation (PBM) systems that integrate one or more of cranial, intranasal, ocular, and peripheral (e.g., abdominal) energy delivery modules with real-time physiological feedback, including electroencephalography (EEG) and/or quantitative EEG (QEEG), heart rate variability (HRV), and peripheral oxygenation (SpO₂). The invention further relates to control architectures employing fractional-order (non-integer order) dynamic models for memory-dependent prediction and iterative parameter optimization of stimulation delivery, and to multi-modal systems combining photonic stimulation with electromagnetic (EM) stimulation and/or with adjunctive metabolic-substrate optimization (including short-chain fatty acids (SCFAs) such as butyrate) to regulate mitochondrial and neural function across central and enteric (gut-brain) domains.

### BACKGROUND OF THE INVENTION

The human brain exhibits significant neuroplasticity, allowing its neural circuits to be shaped and reshaped over time by internal and external stimuli. In recent years, cognitive neuroscience and brain health research have focused on leveraging this plasticity for therapeutic, rehabilitative, and performance-enhancing purposes. A wide range of brain modulation methods has been explored to influence neural function, yet their practical implementations remain constrained by technical and physiological limitations.

Modern neuroscience and cognitive health research have increasingly emphasized the plastic nature of the human brain. It is well established that neural circuits can be reshaped, trained, or modulated through repeated stimulation, therapeutic practices, or behavioral interventions. However, traditional methods for achieving such modulation—whether for relaxation, cognitive enhancement, rehabilitation, or emotional regulation—remain constrained by rigidity, lack of personalization, and absence of real-time feedback.

The field of neurostimulation has evolved significantly over the past decades, encompassing a variety of technologies aimed at altering neural activity for therapeutic or enhancement purposes. These include electrical stimulation techniques such as transcranial direct current stimulation (tDCS), transcranial magnetic stimulation (TMS), electroencephalography (EEG)-based neurofeedback systems, and photobiomodulation methods that utilize specific wavelengths of light to influence brain function. Despite continued development, these technologies suffer from several limitations that restrict their effectiveness, accessibility, and personalization.

Transcranial direct current stimulation (tDCS) is a widely used non-invasive method that delivers low levels of direct electrical current to the scalp to influence cortical excitability. However, tDCS lacks precise targeting and often stimulates large areas of the brain indiscriminately. The effects are highly variable between individuals, and optimal stimulation parameters are not well established. Moreover, the technique generally requires supervision by trained professionals and is not easily adaptable to dynamic changes in brain activity during a session.

Another commonly used technique is transcranial magnetic stimulation (TMS), which involves generating magnetic fields to induce electric currents in the brain. Although TMS has been approved for use in treatment-resistant depression and other psychiatric conditions, it is generally restricted to clinical settings due to the size, cost, and complexity of the equipment. TMS is not easily portable or affordable for most users and does not support continuous at-home application.

EEG-based neurofeedback is a non-invasive technique that provides real-time feedback about a person's brainwave activity with the goal of training the user to alter neural patterns. While promising in theory, neurofeedback systems often require long training periods, are highly dependent on user compliance, and typically do not provide any form of direct stimulation to enhance or reinforce the learning process. Many users struggle to translate visual or auditory feedback into meaningful internal changes, especially without professional guidance.

Photobiomodulation (PBM) has emerged as an alternative approach involving the delivery of light, typically in the red or near-infrared spectrum, to the scalp. These light therapies are believed to influence mitochondrial function and enhance cellular energy production in neurons. However, commercially available PBM systems are limited in their capacity to target specific brain regions or dynamically adjust to real-time brain states. Most PBM systems deliver light uniformly across broad areas of the scalp without regard to functional brain anatomy or individual neurological needs.

Many existing systems in this domain are rigid and offer fixed protocols that do not adapt to individual variability in physiology, mental state, or therapeutic response. These limitations reduce the effectiveness of treatment and often necessitate multiple iterations of trial and error to find suitable parameters for each user.

Further, most devices are designed to operate in a uniform mode of stimulation, typically using either a constant or periodically pulsed emission pattern. This can lead to habituation, where the user's brain adapts to stimulation and becomes less responsive over time. There is a lack of flexibility in stimulation paradigms such as alternating, randomized, or dynamically sequenced patterns that may improve outcomes by preventing neural accommodation.

Additionally, the majority of existing neurostimulation devices do not integrate real-time physiological sensing, such as EEG or heart rate variability (HRV), as inputs to inform or adapt the stimulation protocol. As a result, these devices are unable to personalize treatments based on moment-to-moment changes in the user's cognitive or emotional state.

Accordingly, there remains a significant and long-standing need for an improved, user-friendly, modular, sensor-integrated, and context-aware neurostimulation system that overcomes the shortcomings of the prior art by enabling individualized, adaptive, and safe modulation of brain activity across a wide range of applications and user environments.

Existing commercially available photobiomodulation systems (for example, transcranial and intranasal PBM products such as those marketed by Vielight Inc.) generally rely on a fixed, preconfigured stimulation protocol. Such systems do not dynamically adjust pulse frequency, duty cycle, wavelength selection, intensity, or duration in response to real-time physiological measurements acquired during a session. They are not multi-site systems in the sense contemplated herein (concurrent or coordinated operation across cranial, intranasal, ocular, and abdominal targets), they do not employ memory-dependent fractional-order control models, and they do not couple photonic stimulation to metabolic-substrate optimization (such as endogenous or exogenous SCFA availability). Accordingly, prior art PBM and neurostimulation systems fail to provide closed-loop, multi-domain regulatory operation in which stimulation parameters are continuously and adaptively selected based on measured physiological response across central and enteric domains.

### SUMMARY OF THE DISCLOSURE

Accordingly, the present invention provides systems, apparatuses, and methods for real-time, individualized brain modulation using a wearable device comprising a plurality of energy-emitting and/or sensing portals configured to interact with various regions of the brain through precisely controlled stimulation protocols. The invention addresses long-standing limitations in conventional neurostimulation technologies by enabling targeted, configurable, and adaptive energy delivery informed by real-time physiological and behavioral inputs.

The present invention differs from prior photobiomodulation and neurostimulation systems by incorporating metabolic-substrate optimization and closed-loop physiological feedback to dynamically regulate mitochondrial function across central and enteric domains, and by employing a fractional-order adaptive control engine that converges stimulation parameters toward a target physiological state during the session rather than executing a predetermined stimulation protocol.

In some embodiments, the invention includes a helmet or headgear apparatus configured to be worn over the user's head, wherein the inner surface of the helmet includes multiple zones, each having one or more independently controllable portals. Each portal may comprise one or more energy-emitting elements, one or more sensors, or a combination thereof. The energy-emitting elements may include light-emitting diodes (LEDs), lasers, pulsed light sources, electromagnetic emitters, ultrasonic transducers, or other directed energy devices. The sensors may include EEG sensors, heart rate sensors, temperature sensors, motion sensors, or combinations thereof.

In some embodiments, each portal includes an emitter-sensor pair, wherein the emitter delivers energy to the user's scalp or cranial region, and the sensor detects physiological signals such as brainwave activity or blood flow changes in the corresponding region. In other embodiments, a portal may comprise only a sensor or only an emitter. In yet another embodiment, each portal includes multiple emitters and one sensor, or multiple sensors and one emitter, depending on the intended therapeutic or diagnostic purpose.

In some embodiments, the plurality of portals is arranged in a hemispheric or radial pattern to correspond with specific brain regions, such as the prefrontal cortex, temporal lobes, motor cortex, parietal regions, or occipital cortex. The system may activate specific portals based on user-selected treatment goals, such as focus enhancement, memory improvement, sleep induction, mood regulation, or motor rehabilitation.

In some embodiments, the system may operate in a manual mode in which the user selects a predefined stimulation protocol using a control interface or mobile application. The selected protocol may include settings such as energy type, frequency, intensity, pulse width, duration, and sequence of portal activation. For example, a user may select a “focus mode,” which activates frontal cortex emitters with blue light at 18 Hz for 20 minutes, while suppressing temporal lobe stimulation.

In another embodiment, the system operates in an AI-guided adaptive mode wherein real-time sensor data is processed by a controller running an artificial intelligence engine. The controller analyzes EEG waveforms, heart rate variability, or other physiological inputs to select or adjust the stimulation parameters. For example, if the user's EEG indicates excess theta activity (associated with drowsiness) during a focus session, the controller may shift stimulation frequency to the beta range and increase emitter intensity to maintain alertness.

In some embodiments, the apparatus may include a user interface that supports a diagnostic or interactive questionnaire mode, wherein the system presents a sequence of questions to the user to determine their present cognitive or emotional state. Based on user responses—either through manual input or voice recognition—the system adjusts its emission strategy to deliver personalized therapy. For example, if the user reports anxiety, the system may engage alternating left-right temporal stimulation at calming frequencies combined with heart-rate-synchronized pulsed light.

In yet another embodiment, the system is operable in offline or wireless-free mode using a preloaded control chip embedded within the helmet. This configuration supports environments where electromagnetic field (EMF) exposure is undesirable or prohibited, such as clinical sleep labs or EMF-sensitive user populations. The device may be charged or configured through a USB or similar wired interface.

In some embodiments, the system includes a mode selection switch or touchscreen interface mounted on the helmet surface, allowing the user to toggle between modes such as sleep, meditation, learning, or visual enhancement. Alternatively, the device may connect via a wired or wireless link to a companion mobile application that provides an interactive dashboard for control, progress tracking, and stimulation history logging.

In some embodiments, stimulation patterns may be randomized, alternating, or sequentially rotated among portals to reduce neural habituation and promote distributed plasticity. For example, a session may involve stimulating left parietal, then right prefrontal, then bilateral occipital portals in 10-second intervals with microvariations in frequency and pulse timing. These patterns may be predefined or AI-generated.

In another embodiment, stimulation parameters may be dynamically linked to detected physiological rhythms. For example, emitter pulse frequency may be synchronized to the user's respiratory rate or heartbeat. A slow, rhythmic breathing pattern may result in pulsed light stimulation synchronized with exhalation to reinforce parasympathetic activation and calming effects.

In some embodiments, the helmet includes replaceable or repositionable emitter pods, allowing the user or clinician to configure the system for specific brain targets. These pods may magnetically attach to predefined socket areas or slide into place along a rail system. This configuration supports personalization for neurorehabilitation or developmental neurotherapy.

In yet another embodiment, the invention provides a modular architecture wherein additional pads, strips, or peripheral emitters can be added to the base helmet, for example, for vagus nerve stimulation near the ear, cervical nerve zones, or peripheral muscle feedback integration. These modules may communicate with the main controller via wired or inductive connectors.

In some embodiments, the invention includes safety monitoring subsystems such as thermal sensors or impedance monitors to detect overheating, excessive skin contact pressure, or abnormal resistance. Upon detecting a hazard, the controller may deactivate emitters or reduce power to protect the user.

In another embodiment, the system includes onboard data logging memory that records session details, sensor readings, and stimulation outcomes. These logs may be accessed via a secure application interface or exported for clinical review. Optionally, cloud-based storage may be used for remote monitoring or physician-guided therapy adjustment.

In some embodiments, the invention incorporates a voice-command interface through which the user may initiate, pause, or modify the session without touching the device or app interface. This feature is particularly beneficial for mobility-impaired users or when operating in meditative states.

In some embodiments, the system provides visual or auditory feedback synchronized with stimulation patterns to support entrainment of the user's mental state. For example, during a focus session, a gentle pulsing light pattern may be shown on a smartphone screen in sync with emitter frequencies to reinforce the target cognitive rhythm.

In yet another embodiment, the invention supports multi-user profiles, wherein different users of the same device may access customized settings, session logs, and AI-generated treatment paths based on their unique physiological data and usage history.

In some configurations, the device may integrate motion or posture sensors to detect head tilts, slouching, or movement. The system may pause stimulation during excessive movement or coach the user toward ideal posture, for example, during a deep breathing or meditative session.

In some embodiments, the invention enables the use of gamified biofeedback, wherein users are rewarded for achieving target brain states. For example, if EEG sensors detect a sustained alpha rhythm during a meditation session, the system may trigger visual or auditory rewards, increasing user engagement and long-term adherence.

In some embodiments, the system includes support for therapist-assisted operation. A remote interface may allow a clinician to monitor the user's physiological state, recommend treatment parameters, or push live updates to the control system for real-time adjustment.

In yet another embodiment, stimulation is tailored based on demographic and health parameters such as age, sex, medical history, circadian rhythm preferences, and preexisting neurological conditions. These factors may inform the system's choice of stimulation protocol to improve compatibility and therapeutic value.

In some embodiments, the system is designed to support both acute use (e.g., for pre-exam focus or anxiety reduction) and chronic use (e.g., neuroplasticity enhancement over months). The control software may automatically adjust stimulation goals, intensity levels, and recovery intervals based on session history and observed outcomes.

In some embodiments, the invention allows the user to view real-time brainwave activity on a mobile screen, accompanied by guidance for self-regulation. For example, an overactive beta state may be highlighted with suggestions for breathing techniques or system mode switches.

In another embodiment, the invention may include a wearable eye cover or goggles that deliver targeted photic stimulation to the optic nerve while synchronizing brainwave modulation through cranial portals. This configuration may support visual pathway training or visual-motor coordination.

In some embodiments, the invention is configured to adjust stimulation parameters to specific brainwave bands associated with functional states: delta (1-4 Hz) for sleep, theta (4-8 Hz) for meditative or subconscious access, alpha (8-12 Hz) for relaxation, beta (13-30 Hz) for focus, and gamma (>30 Hz) for memory and learning. The selection may be automatic or user defined.

In yet another embodiment, the system integrates facial expression or voice tone recognition algorithms to assess emotional states such as frustration, calmness, or distraction. These assessments may be used to automatically shift the stimulation pattern in real-time.

In some embodiments, the invention is used in support of specific therapeutic programs including ADHD attention training, PTSD desensitization, post-stroke recovery, or age-related cognitive decline. In each use case, portal placement, energy type, and timing may be customized for optimal results.

In some embodiments, the present invention relates to systems, apparatuses, and methods for adaptive neurostimulation therapy through a head-mounted wearable device configured to interact with a user interface, sensor arrays, and artificial intelligence (AI)-enabled controllers to deliver personalized brain stimulation protocols based on real-time physiological and contextual data. The disclosed invention addresses the growing need for non-invasive, responsive, and modular neuromodulation systems applicable across home, clinical, and remote-care settings.

In one or more embodiments, the invention includes a head-mounted neurostimulation apparatus configured with one or more energy emitter portals, each of which may be selectively activated to deliver targeted stimulation to specific brain regions, including the frontal, parietal, occipital lobes, or cerebellum. The apparatus may include a plurality of integrated sensors for detecting neurological, physiological, or emotional parameters such as EEG signals, heart rate variability (HRV), skin temperature, or facial expression dynamics. A controller embedded within the apparatus is operatively connected to an AI engine, which is programmed to evaluate incoming data in real-time and adaptively adjust stimulation parameters including frequency, emission intensity, duration, and spatial targeting.

The invention further includes multiple configurations for energy emitter portal layouts, including modular segmented portions or a unitary inner shell incorporating emitter arrays across a contoured interior surface. In some embodiments, the apparatus includes removable emitter segments that may be inserted into the outer shell via plug-in connectors and activated via wired or fiber optic links. Alternatively, the system may be implemented as an integrated unit where emitter arrays are embedded within a continuous substrate.

The system supports multiple therapy modes, such as sleep regulation, cognitive enhancement, attention modulation, memory improvement, and relaxation induction. These modes may be initiated manually by the user through a connected user device or activated automatically based on AI-driven assessment of the user's current state. In certain embodiments, the apparatus may be used in standalone mode without requiring constant input from a user device. Wireless communication may be selectively enabled before or after therapy sessions to transmit summary data to a remote server or healthcare provider, thereby reducing electromagnetic exposure during active stimulation periods.

Another feature of the invention is the inclusion of peripheral sensing probes that may be removably attached to the user's body—such as the chest or knees—via wired or wireless connectors. These probes may monitor localized physiological metrics including pulse rate, muscle tension, or skin conductivity and transmit such data to the central controller for therapy refinement.

The invention also supports user interaction through dynamic questionnaires displayed via the user device, as well as emotion recognition through facial expression analysis or vocal tone monitoring. These inputs may supplement biometric sensor data to increase the precision of therapy objective selection. In some implementations, therapy recommendations are displayed as selectable options, or activated automatically when consistent emotional or physiological trends are detected.

Energy stimulation may be deployed across the emitter portals in multiple configurable patterns, including simultaneous activation, alternate firing, or random sequences with variable intensity and frequency. These patterns may be predetermined or generated dynamically by the AI engine based on therapy objectives and session progress.

The invention also incorporates logging and visualization interfaces for tracking user progress across sessions. Metrics such as sleep improvement, cognitive responsiveness, and HRV stability may be recorded and graphed to provide feedback to the user or caregiver. Voice feedback entries and emotion logs may be timestamped and stored for review, analysis, and model training purposes.

In another embodiment, the system enables multi-user functionality, wherein distinct user profiles are stored in encrypted storage and selected via biometric identifiers (e.g., facial recognition, fingerprint, or voice ID) or manual profile switching. Each profile is linked to individualized therapy calibration, progress logs, and configuration preferences, allowing the same apparatus to be shared across multiple users in household or clinical environments.

The invention supports real-time communication with medical professionals or remote monitoring systems. Using the wireless communication module, session data may be transmitted to a remote server for archiving, or to a healthcare professional for live review or intervention. Such architecture supports hybrid therapy delivery models involving professional oversight and autonomous AI-based modulation.

The modularity, personalization, and intelligent control embedded in the present invention offer a scalable and flexible neurostimulation platform that may be used for therapeutic, developmental, or rehabilitative purposes across a broad spectrum of neurological and psychological conditions. Embodiments of the invention may be customized to support adult, pediatric, or geriatric populations, as well as specialized use cases such as post-stroke recovery, attention disorders, or emotional regulation training.

In some embodiments, a neurostimulation apparatus includes a wearable housing designed to be comfortably and securely positioned on the head of a user. The interior surface of the housing supports one or more emitter portions. Each emitter portion includes a plurality of energy emitter portals that are spatially distributed to align with various neuroanatomical regions of the user's head, such as the prefrontal cortex, parietal lobes, temporal regions, or occipital cortex. These portals are operatively configured to deliver stimulation energy, such as modulated light, thermal pulses, or electromagnetic signals, depending on the therapy requirements.

The apparatus further includes a controller housed within the wearable structure. The controller includes a processor, non-transitory memory, and an artificial intelligence (AI) engine. The controller may operatively be connected to the emitter portions and configured to execute intelligent therapy personalization based on real-time and historical data. The AI engine receives input data indicative of the user's current physiological or neurological state through one or more sensors. These sensors may be embedded in the wearable housing or may be part of a set of peripheral modules coupled to the apparatus. The sensor inputs may include electroencephalographic (EEG) signals, galvanic skin response, heart rate variability, temperature, or other biosignals associated with cognitive load, stress levels, or emotional state.

Upon receiving the sensor data, the AI engine references one or more user profiles stored locally or accessed via external sources such as a user device or remote server. Each user profile corresponds to an individual user and includes personalized therapy parameters, historical session data, past physiological responses, and previously applied stimulation patterns. The AI engine analyzes the incoming data in context with the user's profile to determine a customized therapy configuration. The configuration may include a selection of one or more emitter portions, designation of specific emitter portals, definition of stimulation frequencies, determination of emission intensities, choice of stimulation patterns (such as simultaneous, alternating, or pseudo-random), and assignment of session duration.

Following the generation of the therapy configuration, the controller activates the designated energy emitter portals in accordance with the AI engine's instructions. Each emitter portal may be individually addressable, allowing fine-grained control of stimulation across the entire head surface. The stimulation energy may be continuously or intermittently modulated throughout the session.

The apparatus is also configured to support multi-user operation. Upon initial interaction, the user may be identified using biometric data or manual input. Biometric identification may include voiceprint recognition through an onboard microphone, facial scanning via a paired camera, or fingerprint authentication using a sensor on a connected device. Once the user is identified, the controller retrieves the corresponding user profile, either from local memory or via a wired or wireless link to an external device or secure server. This allows multiple individuals to use the same apparatus without compromising personalized therapy continuity.

The apparatus may connect to a user device such as a smartphone or tablet via USB, Bluetooth, or other communication protocols. The user device may be used to display therapy options, visualize session metrics, receive user input, or provide feedback. In some embodiments, the user device is capable of executing supplementary AI-based analysis to augment the decision-making process of the onboard controller or provide cloud-based processing where deeper neural models may be employed.

Additionally, the system incorporates a questionnaire interface accessible through the user device. This interface presents a series of therapy-related questions that adapt based on the user's responses. For example, if the user indicates elevated stress levels, the system may follow up with additional queries to determine whether the stress is cognitive, emotional, or physiological. The input from this dynamic questionnaire is processed by the controller and contributes to the determination or adjustment of the therapy configuration.

Throughout the session, the AI engine dynamically evaluates ongoing data streams from the sensors and any user interaction detected. If the system detects a deviation from the expected response—for example, increased physiological arousal during a calming routine—it may immediately adjust parameters such as intensity, frequency, or the active emitter region. This closed-loop feedback mechanism allows the apparatus to deliver therapy that is continuously tailored to the user's state in real time, maximizing therapeutic impact while minimizing overstimulation or adverse reactions.

For example, during a cognitive enhancement routine, if the user begins showing signs of overstimulation in the prefrontal area, the AI engine may reduce the intensity in that region and shift stimulation to the parietal regions at a lower frequency. If the session is intended to aid sleep and the AI detects increasing EEG beta wave activity (indicating alertness), it may activate emitter portals near the occipital lobe with low-frequency infrared pulses while triggering calming audio through an onboard or connected speaker.

The apparatus may also log each session's configuration, biometric responses, and AI decisions to the user profile for ongoing personalization. These logs may be stored locally or synchronized to a remote server for access by healthcare professionals, researchers, or for use in training future iterations of the AI model. The system supports secure, encrypted communication to protect sensitive health-related data.

In some embodiments, a method for delivering personalized neurostimulation therapy to a user begins with positioning a wearable neurostimulation apparatus onto the head of the user. The apparatus includes an interior surface that supports one or more emitter portions, each emitter portion comprising a plurality of energy emitter portals. These portals are strategically arranged to target different brain regions and are capable of delivering stimulation energy in various modalities including optical (e.g., infrared, near-infrared, visible light), thermal, or vibrational forms depending on the therapy design.

The method proceeds by receiving one or more user inputs through an integrated or connected input interface. The user input may include, for example, a manual selection made via a physical control button, touchscreen interface, or a user device application. Additionally, inputs may be received as voice commands processed by a microphone and interpreted using voice recognition algorithms. In other scenarios, the user may be identified and authenticated based on biometric input such as facial recognition, voiceprint, or fingerprint scan. The user may also be prompted with a dynamic questionnaire, and the responses provided by the user may further influence the configuration of the therapy session.

The received input is processed by a controller embedded within the apparatus. The controller includes a processor, a memory module, and an artificial intelligence (AI) engine. The AI engine is tasked with analyzing the input data to determine a personalized therapy configuration for the session. This configuration may include determining which emitter portions or emitter portals should be activated, selecting an optimal stimulation frequency for the user's condition, deciding on the energy emission intensity, defining the stimulation pattern (such as alternating or simultaneous pulses), and setting the total duration of the session.

Once the therapy configuration has been determined, the controller activates the selected emitter portals. Each portal may be driven by its own control signal to enable independent operation, or multiple portals may be grouped for coordinated stimulation across specific regions. The energy delivery may begin with a pre-programmed baseline or be modulated from the onset depending on the input data.

As the session progresses, the AI engine continues to operate in a closed-loop fashion. The system receives ongoing input from one or more onboard sensors and optionally from peripheral sensing probes. Based on the continuous analysis of sensor feedback, user interaction (such as physical responses or real-time commands), emotional state estimation, or observed therapy progression, the controller modifies the ongoing therapy configuration. For example, if sensor input indicates overstimulation or physiological stress, the AI may reduce the stimulation frequency or deactivate selected portals to prevent adverse effects.

In some implementations of the method, the emitter portions of the apparatus may be provided as removably attachable modules. During setup, an emitter portion may be selected and attached based on the therapy's target region—such as the prefrontal cortex for cognitive therapy or the occipital lobe for relaxation. This modularity allows clinicians or users to customize the device layout based on therapy goals or anatomical preferences.

Further, the method may include the step of receiving physiological data from one or more peripheral sensing probes. These probes may be positioned on non-head regions of the user's body such as the chest, forearm, wrist, or leg. The probes may be configured to detect various physiological parameters relevant to therapy adjustment. These parameters include heart rate variability (HRV), which may indicate stress or parasympathetic response; skin temperature, which may fluctuate in response to emotional arousal; and galvanic skin response (GSR), which provides a measure of electrodermal activity often correlated with alertness or stress.

As an example of the method in use, a user preparing for a cognitive enhancement session places the apparatus on their head and launches the companion app on a smartphone. The app presents a few targeted questions, and the user provides answers indicating mental fatigue and upcoming high-focus tasks. The system automatically selects an emitter portion aligned with the prefrontal region. Peripheral probes positioned on the user's wrist begin collecting real-time HRV and GSR data. The AI engine determines a therapy profile of alternating high-frequency pulses directed to the prefrontal portals, with a ramp-down in intensity after the first 10 minutes.

As the session continues, sensor data indicates a drop in HRV and increased GSR, suggesting elevated sympathetic activity. In response, the AI dynamically reduces stimulation frequency, adds low-frequency pulses to the occipital region, and plays a synchronized tone through an optional speaker to induce relaxation while maintaining focus. The session continues with real-time adjustments until completion, after which all data is logged and optionally transmitted to a remote server or healthcare provider for review.

The disclosed invention thus offers a comprehensive and modular brain modulation system that can operate in real-time, adaptively respond to user physiology, and support a wide range of therapeutic, cognitive, and behavioral goals. The ability to customize portal configuration, stimulation type, and control logic enables broad applicability and marks a significant advancement over prior neurostimulation technologies.

In some embodiments, the invention provides an adaptive multi-site neurostimulation system comprising one or more of: (i) a cranial photobiomodulation (PBM) module; (ii) an intranasal stimulation module configured to deliver light energy into a nasal cavity of the user; and (iii) one or more peripheral stimulation modules configured for placement on the abdomen, chest, limbs, or cervical region, wherein the system coordinates stimulation across two or more of said modules based on real-time physiological signals.

In some embodiments, the intranasal stimulation module includes an insertable probe configured to emit near-infrared light at approximately 810 nm and/or approximately 1070 nm, with an output power of at least about 50 mW, and in particular embodiments up to about 80 mW. The intranasal stimulation module may include one or more interchangeable probe tips, one or more thermal sensors configured to regulate safe operation within nasal tissues, and a retention or alignment feature configured to position the probe relative to the cribriform plate, the olfactory mucosa, or the sphenoidal recess.

In some embodiments, the system acquires EEG signals (including quantitative EEG (QEEG) features such as dominant frequency, amplitude, variability, and inter-channel coherence) at sampling rates sufficient to extract said features in real time, and concurrently acquires HRV data and SpO₂ data to provide autonomic, cardiovascular, and oxygen-utilization indicators of the user's state.

In some embodiments, the system includes a controller executing a closed-loop adaptive algorithm wherein stimulation parameters—including pulse frequency, duty cycle, intensity, waveform, wavelength selection (e.g., 810 nm versus 1070 nm), site selection (cranial versus intranasal versus peripheral), and session duration—are iteratively adjusted during the session based on the measured physiological response. The stimulation parameters are dynamically selected and updated based on the measured physiological response rather than execution of a predetermined stimulation protocol.

In some embodiments, the controller employs a fractional-order (non-integer order) dynamic model characterized by one or more non-integer order differential equations representing memory-dependent system behavior. The model is configured to (a) predict a physiological response of the user to a stimulation input, (b) compare the predicted response to a measured physiological response to generate an error signal, and (c) update the stimulation parameters to reduce the error signal over successive iterations, the model being continuously updated based on observed data.

In some embodiments, the stimulation parameters include pulsed frequencies such as approximately 10 Hz (alpha-band entrainment), approximately 40 Hz (gamma-band, with relevance to neurodegenerative disorders), approximately 48 Hz, approximately 432 Hz, and additional non-linear frequencies; duty cycles between approximately 30% and approximately 90%; and session durations between approximately 15 and approximately 60 minutes.

In some embodiments, the system may coordinate stimulation across multiple sites, including simultaneous or alternating activation of cranial and intranasal modules, and may further integrate electromagnetic (EM) stimulation modalities synchronized with photonic stimulation. Coordination of intranasal and cranial stimulation provides synergistic central nervous system engagement (via the olfactory and trans-cranial pathways), and addition of abdominal PBM permits regulation of gut perfusion, microbiome composition, and SCFA production, thereby engaging the gut-brain axis.

In some embodiments, the system implements a dual-input energetic regulation model wherein photobiomodulation provides a mitochondrial efficiency input and metabolic substrates (for example, butyrate or other SCFAs, whether endogenously produced via abdominal PBM-driven microbiome modulation or exogenously administered) provides a metabolic substrate input. This dual-input architecture provides a two-axis control over cellular energetics that supports mitochondrial regulation in central and enteric tissues.

In some embodiments, the controller is further configured to store historical physiological data and stimulation outcomes across sessions, and to use the stored data to initialize subsequent sessions and to improve parameter selection across sessions (a learning-across-sessions function).

### BRIEF DESCRIPTION OF THE DRAWINGS

The accompanying drawings, which are incorporated into and constitute a part of this specification, illustrate several embodiments of the present invention. Together with the description, these drawings serve to illustrate some aspects of the present invention.

FIG. 1 illustrates a perspective view of a head-mounted wearable apparatus, in accordance with some embodiments of the present invention.

FIG. 1A illustrates exemplary configurations of energy emitter portals, each configuration comprising one or more energy emitters and one or more sensors arranged within a portal, in accordance with various embodiments of the present invention.

FIG. 1B illustrates an exemplary head-mounted neurostimulation apparatus comprising a unitary inner shell structure having a plurality of integrated energy emitter portals, in accordance with various embodiments of the present invention.

FIG. 2 illustrates an exploded view of a head-mounted wearable apparatus showing a plurality of interchangeable modular emission inserts configured for energy delivery within the apparatus, in accordance with some embodiments of the present invention.

FIG. 3 illustrates an exploded view of a modular head-mounted wearable apparatus comprising a plurality of removable emitter portions, each having a respective array of energy emitter portals, in accordance with some embodiments of the present invention.

FIG. 4 illustrates a head-mounted neurostimulation apparatus connected to a user device, wherein the user device displays a plurality of therapy options and stimulation parameters, in accordance with various embodiments of the present invention.

FIGS. 4A-4D illustrate various user interface modes associated with a head-mounted neurostimulation apparatus, in accordance with various embodiments of the present invention.

FIG. 4E illustrates an exemplary head-mounted neurostimulation apparatus configured for voice-based operation, in accordance with some embodiments of the present invention.

FIG. 5 illustrates a head-mounted neurostimulation apparatus configured to target different brain regions, with a user device displaying corresponding therapy areas and stimulation parameters including region-specific activation frequencies, in accordance with various embodiments of the present invention.

FIG. 5A illustrates a schematic view of a head-mounted neurostimulation apparatus showing targeted application of energy emitters to specific brain regions, wherein different energy emitters may be selectively activated based on the therapy requirements.

FIG. 6 illustrates an exemplary head-mounted neurostimulation apparatus being operatively connected to a user device and a plurality of peripheral sensing probes positioned on a user's body, in accordance with some embodiments of the present invention.

FIG. 7 illustrates an exemplary head-mounted neurostimulation apparatus comprising a goggle-type wearable device configured with a plurality of energy emitters positioned over the ocular and facial regions, in accordance with some implementations of the present invention.

FIG. 8 illustrates an exemplary neurostimulation apparatus comprising a helmet portion and a removably attachable goggle portion, in accordance with some embodiments of the present invention.

FIG. 9 illustrates an exemplary neurostimulation goggle configured with a see-through lens portion and a surrounding energy emitter portion to deliver targeted stimulation to the ocular and periorbital regions, in accordance with some embodiments of the present invention.

FIG. 10 illustrates a block diagram of an exemplary neurostimulation apparatus, including a controller configured with an AI engine and operatively coupled to various functional components, in accordance with various embodiments of the present invention.

FIGS. 11A-11B illustrate exemplary method steps that may be executed in some embodiments of the present invention.

FIG. 12 illustrates an exemplary multi-user profile management system for a neurostimulation apparatus, in accordance with some embodiments of the present invention.

FIG. 13 illustrates an exemplary communication architecture in which a neurostimulation apparatus interfaces with one or more user devices, a remote server, and a medical professional, via wired and/or wireless connections, enabling session control, data exchange, remote monitoring, and professional intervention, in accordance with various embodiments of the present invention.

FIG. 14 illustrates exemplary stimulation pattern variants including simultaneous, alternate, and random energy emission patterns, demonstrating variable intensity and frequency modulation across emitter portals or portions, in accordance with some embodiments of the present invention.

FIG. 15 illustrates an exemplary user progress and feedback logging interface, showing tracked metrics such as sleep, cognition, and HRV stability across multiple sessions, along with emotion recognition and timestamped voice feedback, in accordance with various embodiments of the present invention.

FIG. 16 illustrates a high-level system architecture of an adaptive multi-site neurostimulation system, showing a cranial photobiomodulation module, an intranasal stimulation module, one or more peripheral stimulation modules, a sensor subsystem, and a controller embodying a closed-loop adaptive engine, in accordance with various embodiments of the present invention.

FIG. 17 illustrates a closed-loop control diagram of the adaptive neurostimulation system, including signal acquisition (EEG/QEEG, HRV, SpO₂), feature extraction, an adaptive control engine, stimulation output, and a feedback path used to iteratively modify stimulation parameters during a session, in accordance with various embodiments of the present invention.

FIG. 18 illustrates a schematic of a fractional-order dynamic model used by the controller, including representation of non-integer order differential operators, memory-dependent state, predicted-versus-measured response comparison, error generation, and parameter update, in accordance with various embodiments of the present invention.

FIG. 19 illustrates an intranasal stimulation device, including a probe body, one or more interchangeable distal tips configured for light delivery (for example, at approximately 810 nm and/or approximately 1070 nm), a thermal sensor, retention or alignment feature, and a connector configured to interface with the controller, in accordance with various embodiments of the present invention.

FIG. 20 illustrates a parameter optimization layer in which stimulation parameters (frequency, duty cycle, intensity, wavelength, duration) are progressively adjusted based on measured physiological response surfaces, in accordance with various embodiments of the present invention.

FIG. 21 illustrates multi-modal integration in which photobiomodulation, electromagnetic stimulation, and metabolic-substrate optimization (for example, butyrate or other SCFAs) operate as concurrent inputs to a dual-input energetic regulation model targeting central and enteric domains, in accordance with various embodiments of the present invention.

FIG. 22 illustrates a learning-across-sessions curve in which one or more outcome metrics (for example, prediction error, time-to-target physiological state, or therapeutic response score) decrease over successive sessions as the controller refines stimulation parameters and updates its stored fractional-order model, in accordance with various embodiments of the present invention.

### DETAILED DESCRIPTION

The present invention provides systems, methods, and apparatus for delivering personalized neurostimulation therapy to a user. In some embodiments, a neurostimulation apparatus includes a wearable housing configured to be positioned on the head of a user to facilitate targeted delivery of stimulation energy. The wearable housing may be constructed of lightweight, ergonomically contoured material with internal padding to conform to various head shapes and sizes. The housing may support modular integration of one or more emitter portions mounted on its interior surface. Each emitter portion may include a plurality of energy emitter portals configured to deliver stimulation energy to specific anatomical regions of the user's head, including the frontal lobe, parietal region, occipital region, and cerebellum.

Some embodiments include an adaptive multi-site neurostimulation system and method for delivering personalized, closed-loop stimulation therapy to a user based on real-time physiological feedback. The system includes one or more stimulation modules—including a cranial photobiomodulation module, an intranasal stimulation module configured to deliver near-infrared light (for example, at approximately 810 nm and/or approximately 1070 nm, at outputs up to about 80 mW, via interchangeable probe tips with thermal sensing), and one or more peripheral stimulation modules including an abdominal photobiomodulation applicator—together with a sensor subsystem acquiring EEG/QEEG, HRV, and SpO₂ signals, and a controller. The controller executes a closed-loop adaptive engine in which a fractional-order, memory-dependent dynamic model predicts physiological response and iteratively updates stimulation parameters, frequency (for example, approximately 10 Hz, 40 Hz, or 432 Hz), duty cycle (for example, approximately 30-90%), intensity, waveform, wavelength selection (810/1070 nm), site selection, and duration (for example, approximately 15-60 minutes)—to reduce a prediction error and converge toward a target physiological state. The system further integrates metabolic-substrate optimization (including short-chain fatty acids such as butyrate, modulated endogenously via abdominal PBM influence on the gut-brain axis or administered exogenously) as a co-input to a dual-input energetic regulation of mitochondrial and neural function across central and enteric domains, and learns across sessions by storing and refining the fractional-order model.

The energy emitter portals may include light emitters capable of emitting in one or more selected wavelength ranges such as visible light, infrared, or near-infrared. In some configurations, individual energy emitter portals may also include one or more sensors to detect physiological signals, including, but not limited to EEG signals, skin temperature, or hemodynamic responses. In this dual-function design, the portal may function as both a stimulation emitter and a localized sensor, enhancing the system's ability to perform closed-loop feedback therapy.

The apparatus may further comprise a modular architecture such that one or more of the emitter portions are removably attachable. This enables replacement or customization of emitter modules for different users or therapeutic goals. The modular emitter portions may be available in multiple shapes and sizes, each optimized for targeted coverage of specific cortical regions.

A controller embedded within the apparatus may include a processor, memory, and an artificial intelligence (AI) engine. The controller is operatively coupled to the emitter portions and input interface. The AI engine is configured to process one or more user inputs to determine a personalized therapy configuration. Inputs may include manual selections via a touchscreen interface, voice commands received via a microphone, biometric identifiers such as fingerprint or facial recognition, or responses to a dynamic questionnaire presented via a user device. In some implementations, the questionnaire may include a sequence of therapy-related questions whose content and order are adapted in real time based on the user's prior responses.

Once user input is received, the AI engine may determine a therapy configuration that includes parameters such as which emitter portions or emitter portals are activated, the stimulation frequency, emission intensity, emission pattern, and the total duration of the stimulation. The emission pattern may include a simultaneous emission across multiple portals, an alternating pattern in a specified sequence, or a pseudo-random pattern to minimize habituation and maximize therapeutic effect.

The AI engine may also be configured to identify the user from among a plurality of stored profiles using biometric or manual selection methods. Once identified, a user profile may be retrieved from internal memory, an external user device, or a remote server. The user profile may include personalized therapy parameters and historical session data. The apparatus may connect to the user device via a wired or wireless interface. The user device may be configured to display therapy options, collect real-time feedback, visualize stimulation metrics such as intensity and duration, or transmit data to and from a remote server or medical platform.

In one example, the apparatus may be used by a user experiencing high stress. The user may initiate the session using voice input, and the apparatus may respond by verifying the user's identity via a stored voiceprint. A dynamic questionnaire presented on the user's smartphone may assess the user's emotional state. Based on the responses and baseline physiological data obtained via integrated sensors and/or peripheral probes (e.g., heart rate variability measured via a chest probe), the AI engine may determine that a calming stimulation pattern applied primarily to the parietal and occipital regions is optimal. The emitter portals in these regions may then be activated using a randomized, low-frequency infrared stimulation at gradually decreasing intensity.

The apparatus may also support optional audio feedback via an integrated speaker. The speaker may deliver synchronized auditory cues, including relaxing tones or guided meditation tracks, matched in rhythm with the energy stimulation pattern. The microphone may concurrently monitor user vocal output for changes in pitch, pace, and tone as indicators of stress reduction or emotional shift, allowing the AI engine to make real-time adjustments to stimulation parameters.

Additional peripheral components may be connected via a physical port located on the wearable housing. These may include removable external sensing probes configured to be placed on the user's chest, arm, or leg, for monitoring physiological indicators such as blood circulation, respiration, skin conductance, or temperature. A goggle module may also be attached to deliver light stimulation to the periorbital region, without obstructing the user's vision. This may be used in protocols targeting circadian rhythm regulation, mood enhancement, or visual processing rehabilitation.

In more advanced use cases, the apparatus may employ emitter portals capable of operating at different frequencies simultaneously. For example, emitter portals directed at the frontal region may deliver high-frequency stimulation to support attention, while those near the occipital region may deliver lower-frequency stimulation to promote relaxation. Such configurations are determined and managed by the AI engine, which continuously evaluates user status via feedback from onboard sensors and adjusts the therapy parameters accordingly.

The stimulation parameters may also be modified in real time based on therapy progression. If the user begins to show signs of cognitive fatigue or emotional discomfort during a session, the AI engine may reduce the stimulation intensity or reconfigure the activation sequence to prioritize recovery. This closed-loop adjustment is based on a combination of historical session data, real-time sensor inputs, and AI-determined projections of therapeutic response.

The apparatus may function in standalone mode or in conjunction with a user device such as a smartphone or tablet. In standalone mode, the apparatus operates based on preset therapy routines and internal AI processing. In connected mode, the user device provides a larger interface for user interaction, cloud syncing, therapy scheduling, and external monitoring. Session logs, performance metrics, and recommendations may be visualized on the user device, aiding in long-term therapy tracking and outcome analysis.

In some embodiments, the neurostimulation apparatus includes a wearable housing configured to be placed over the user's head, the housing enclosing an internal compartment that contains the controller and associated circuitry. The internal compartment may be electromagnetically shielded to minimize interference from external or internal wireless signals and to preserve the fidelity of real-time physiological sensing. The apparatus may be powered by an integrated rechargeable battery module, allowing untethered use across multiple therapy sessions.

The apparatus includes one or more emitter portions, with at least one embodiment comprising a single emitter portion lining the interior surface of the wearable housing. This single emitter portion may incorporate a dense array of energy emitter portals configured to deliver stimulation to various regions of the head. The emitter portion may be flexible or contoured to accommodate the natural curvature of different skull shapes. Multiple sizes of such emitter portions may be made available, with interchangeable inserts tailored to users with different head size categories, improving fit, contact reliability, and stimulation accuracy.

Each energy emitter portal within the emitter portion is individually addressable and controllable by the controller. The portals are configured to deliver stimulation energy using a variety of waveform types, including pulsed waveforms, sine waves, square waves, or custom-modulated patterns. This enables precise tailoring of the stimulation profile to match therapeutic goals. In certain implementations, different portals may operate simultaneously at distinct frequencies to stimulate various brain regions in parallel. For example, a high-frequency stimulation pattern could be delivered to the frontal cortex to promote focus, while low-frequency emissions may be used at the occipital region for relaxation.

The controller includes a processor and memory, and is integrated with an artificial intelligence (AI) engine configured to analyze various forms of input and sensor data. Prior to the beginning of a session, the AI engine may receive one or more user inputs via the input interface—such as biometric identifiers (e.g., voiceprint, fingerprint, or facial scan), responses to a dynamic therapy questionnaire, or manually selected session goals. The controller uses this input to generate a personalized session-specific therapy protocol, including selection of emitter regions, frequencies, intensities, and waveform patterns.

The questionnaire interface may dynamically adapt the types of questions presented based on the selected therapy category. For example, if the user selects a cognitive enhancement protocol, the interface may prioritize questions related to focus, memory, and mental clarity. For stress-related routines, the questionnaire may inquire about recent emotional events, physical symptoms of anxiety, or sleep disturbances. The controller is configured to interpret the answers, build a therapy configuration accordingly, and continuously refine the system's predictive accuracy across sessions.

During therapy, the AI engine may monitor data streams from onboard sensors and optional peripheral probes to classify the user's current neurological or emotional state. These inputs may include EEG signals from integrated electrodes, galvanic skin response, heart rate variability, or body temperature. The AI engine may additionally interpret facial muscle movement via connected vision systems, and voice tone or cadence captured through the microphone, to estimate mood or stress levels.

The AI engine is configured to modify the therapy configuration in real time based on detected emotional or physiological transitions. For example, if the system detects increased sympathetic nervous system activation, the controller may lower stimulation intensity or transition to a calming frequency pattern. In another scenario, detection of high user engagement or focus might prompt the system to increase stimulation in the prefrontal regions. To maintain a safe experience, the AI may suppress or deactivate certain emitter portals in response to sensor feedback indicating overstimulation or localized discomfort.

For real-world usability, the wearable housing is also equipped with one or more external control buttons for manual operation, allowing the user to initiate, pause, or terminate therapy sessions without accessing a connected device. The controller may disable wireless transmission functions during an active session to reduce possible electromagnetic interference with neural activity and sensing. After completion of the session, data from the therapy may be logged locally and optionally transmitted via a data transmission module to a remote server, cloud system, or authorized medical professional.

In clinical or remote care applications, the controller may be configured to receive therapy parameters directly from a healthcare provider, either through manual entry on the user device or via remote communication. This enables a physician to configure therapy settings for patients with limited autonomy or for those enrolled in monitored rehabilitation programs. When operating in standalone mode without an internet connection, the apparatus can rely entirely on locally stored AI models and user data to make decisions, enhancing its utility in privacy-sensitive environments or areas with limited connectivity.

In the following sections, detailed descriptions of examples and methods will be given. The description of both preferred and alternative examples, though thorough, are exemplary only. It is understood by those skilled in the art, that various modifications and alterations may be apparent and within the scope of the present invention. Unless otherwise indicated by the language of the claims, the examples do not limit the broadness of the aspects of the underlying invention as defined by the claims.

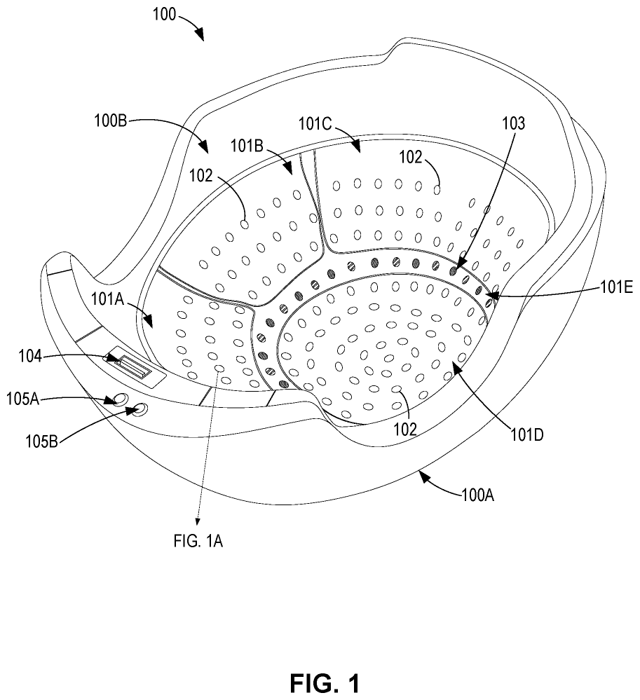

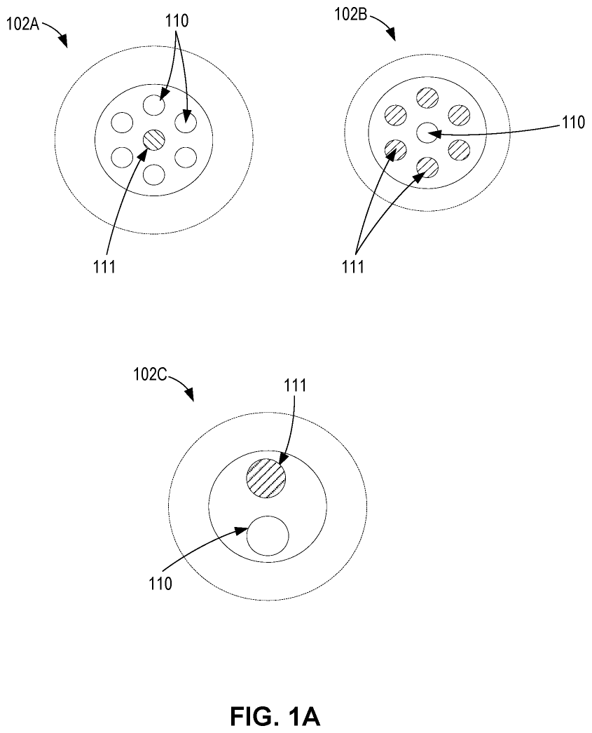

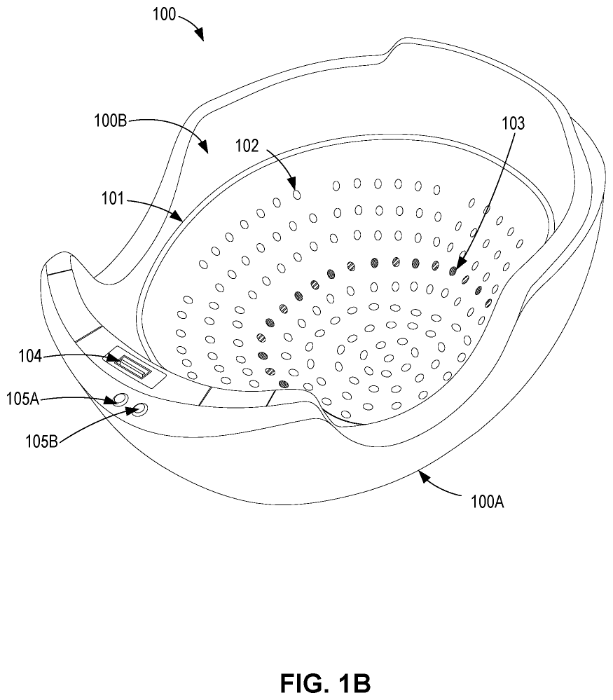

Referring to FIG. 1, a perspective view of a head-mounted wearable apparatus 100 is illustrated, which may include a plurality of emitter-sensor portals 102 distributed across its inner surface for neurostimulation and sensing, in accordance with various embodiments of the present invention. The apparatus 100 may comprise an outer shell 100A and an inner cavity 100B (wearable housing) designed for accommodating the head of a wearer. The outer shell 100A may be formed from durable, lightweight materials such as high-impact polycarbonate, acrylonitrile butadiene styrene (ABS), or carbon-reinforced polymers. In some embodiments, thermoplastic polyurethane (TPU) or silicone-based materials may be used for flexibility and comfort.

The inner cavity 100B may be shaped to conform to the general curvature of a human head and may feature various geometries across different product variants to support users of different age groups, cranial sizes, or therapeutic goals. The inner cavity 100B may be lined with a cushioned support layer or a breathable mesh to facilitate prolonged use. Further, the inner cavity 100B may function as the structural interface for receiving and securing a plurality of emitter portions 101A, 101B, 101C, 101D, and 101E.

Each emitter portion 101A through 101E may be arranged to target a specific region of the user's cranial anatomy. Emitter portion 101A may be positioned to correspond with the frontal region of the user's head and may be particularly configured to stimulate the prefrontal cortex, which is commonly associated with decision-making, focus, and executive function. Emitter portion 101B may be positioned to extend along the left parietal region, while emitter portion 101C may correspond to the right parietal region. Emitter portion 101D may generally be configured along the occipital contour of the skull to influence visual processing areas. Emitter portion 101E may be centrally located to cover the top of the skull or regions involved in motor planning.

Each of the emitter portions 101A through 101E includes a plurality of energy emitter portals 102. These energy emitter portals 102 may include light-emitting diodes (LEDs), laser diodes, or other suitable energy sources configured to emit electromagnetic energy in specific wavelength bands, such as red light (630-670 nm), near-infrared (780-850 nm), or other ranges appropriate for transcranial photobiomodulation. In some embodiments, the energy emitter portals 102 are configured to emit waveforms selected from: pulsed, square wave, sine wave, or custom modulated patterns.

In some embodiments, the energy emitter portals 102 may also include embedded sensors capable of detecting one or more physiological parameters. These sensors may include electroencephalogram (EEG) electrodes, photoplethysmographic (PPG) sensors, skin temperature sensors, galvanic skin response (GSR) sensors, and accelerometers. EEG sensors may be utilized for detecting electrical brain activity in real time, allowing the system to adapt stimulation patterns based on sensed neurological states.

The emitter portions 101A through 101E may have tailored shapes and densities of energy emitter portals 102, depending on the therapeutic or diagnostic requirements. For example, emitter portion 101A, targeting the frontal cortex, may include a dense matrix of LED-based energy emitter portals 102 to provide distributed low-level light therapy (LLLT) across the dorsolateral prefrontal cortex. Emitter portion 101D, aligned to the occipital lobe, may have fewer but higher-intensity portals for visual pathway stimulation.

In some configurations, the energy emitter portals 102 may comprise hybrid structures where light emitters and biosensors are positioned side-by-side within the same portal footprint. Alternatively, some portals may be dedicated exclusively to emission, while others may be reserved for sensing. In some embodiments, emitter portion 101E may comprise only sensors 103, such as high-resolution EEG nodes or dedicated temperature and hydration sensors, to serve as a real-time monitoring zone.

Dedicated sensors 103 may be configured to measure localized electrical activity, temperature fluctuations, skin conductivity, or vascular response, and may be specifically tuned to detect variations in cognitive workload, emotional state, or neurological anomalies. In therapeutic applications, sensors 103 located in emitter portion 101E may support feedback-driven motor rehabilitation by monitoring muscle activation patterns via electromyography (EMG) or tracking motion using inertial measurement units (IMUs).

In some embodiments, the sensors integrated within the energy emitter portals 102 may differ from the dedicated sensors 103 in type, function, resolution, or data sampling rate. For example, a sensor within an energy portal 102 may be configured for basic HRV or alpha wave detection, whereas sensor 103 may be configured for multi-channel EEG acquisition, motion tracking, or real-time event logging for neurological trend analysis.

The apparatus 100 may further comprise a control and processing assembly embedded within or mounted to the outer shell 100A. This assembly may include a microcontroller, system-on-chip (SoC), or digital signal processor (DSP) configured to manage energy emission cycles, acquire sensor data, and execute closed-loop control algorithms. Memory modules may be provided for storing session data, user profiles, and firmware instructions.

Printed circuit board (PCB) assemblies may be integrated into one or more interior or exterior regions of the apparatus 100 for electrical interconnection of the emitter portals 102, sensors 103, and user interface components. The PCB may support high-speed signal routing, analog-to-digital conversion, and power distribution networks to energize the emitter arrays and sensor modules.

The apparatus 100 may include wireless communication circuitry, such as Bluetooth Low Energy (BLE), Wi-Fi, ZigBee, or near-field communication (NFC), to interface with external computing devices, mobile phones, or cloud-based platforms. In some embodiments, the wireless circuitry may remain disabled during active sessions to reduce electromagnetic interference or user sensitivity to wireless exposure.

An external connection port 104 may be located along the surface of the outer shell 100A. This port may comprise a USB-C, micro-USB, or magnetic interface for charging the apparatus 100 or for establishing a direct data link with external devices such as smartphones, tablets, or desktop computers. This physical connection may also allow firmware updates, session downloads, or calibration procedures to be performed.

The apparatus 100 may include one or more physical interface buttons 105A and 105B disposed on the outer shell 100A. Button 105A may serve as a primary power switch for turning the device on or off, while button 105B may be configured to cycle through stimulation modes or initiate wireless pairing. In some embodiments, button 105B may toggle between therapy presets or enable voice command functionality.

Voice activation support may be provided via a microphone interface (not shown in this figure) coupled to the processing unit. This voice interface may be used to receive verbal commands from the user to initiate, pause, or modify therapy settings, allowing for hands-free control in various use environments.

In certain configurations, the apparatus 100 may include onboard storage of therapy programs that can be manually or automatically activated based on sensed brainwave patterns, user behavior, or external command inputs. These therapy programs may include settings for session duration, emission frequency, waveform characteristics, and sensor sampling intervals.

The outer shell 100A of the apparatus 100 may be shaped in various helmet-like forms, including full cranial enclosures, open-top bands, or rear-skull braces, depending on use case. While the figure shows a full-coverage configuration, alternate embodiments may provide modular form factors suited for localized stimulation, such as frontal-only or occipital-only variants.

The inner liner shell 100B may be fabricated as a removable or replaceable component to allow for different fits or to swap different emitter-sensor configurations. In some embodiments, inner liner shell 100B may be magnetic or clip-mounted to the outer shell 100A, enabling ease of disassembly and cleaning.

Each emitter portion 101A-101E may be detachably mounted to the inner liner shell 100B using mechanical fasteners, flexible printed connectors, or spring-loaded contacts. This modular design may allow the user or technician to replace, upgrade, or reposition emitter portions based on individual therapy goals or wear patterns.

The shape, size, and density of emitter portals 102 within each emitter portion 101A-101E may be customized for different demographics. For example, pediatric versions may include smaller emitter arrays with lower energy intensity, whereas rehabilitation-focused designs may prioritize high-density sensors in motor-associated areas.

In further embodiments, each emitter portion 101A-101E may support multiple energy modalities such as pulsed infrared, visible red, or blue light for skin-surface stimulation. In another embodiment, ultrasonic transducers or microcurrent emitters may be embedded within selected portals 102 to facilitate deeper penetration or non-photonic therapies.

To support user safety, the apparatus 100 may be configured to self-monitor electrical output, thermal performance, and sensor data consistency. Alerts may be generated in case of abnormal sensor readings, excessive heat buildup, or disconnection of any emitter portion. The inner surface of the apparatus 100 may be designed to maintain stable contact with the scalp without creating pressure hotspots. This may be achieved by alternating the placement of firm emitter portions 101A-101E with flexible cushioning regions or compliant gaskets.

The orientation and angle of energy emitter portals 102 within each emitter portion 101A-101E may be designed to direct energy perpendicularly to the scalp surface, or at a specific angle to reach cortical regions more effectively. Adjustable mounts or pivoting joints may be incorporated for this purpose. Energy emitter portals 102 may be capable of emitting programmable waveforms, such as sinusoidal, square, or modulated pulses. These waveforms may be tuned to optimize absorption by different tissue depths or neuroreceptive profiles.

The sensor data collected from sensors 103 or those embedded in portals 102 may be processed using onboard machine learning models trained to classify brain states such as focused attention, drowsiness, relaxation, or emotional stress. Therapy output may be dynamically adjusted based on these classifications. In one example embodiment, sensor 103 within emitter portion 101E may detect reduced alpha band activity, prompting the system to activate frontal emitter portion 101A at a higher pulse frequency to stimulate attentional control regions.

In another embodiment, accelerometer data collected from a sensor embedded in emitter portion 101C may detect head nodding associated with fatigue. The system may respond by increasing light intensity in emitter portion 101D to activate alertness pathways. Apparatus 100 may be used in a wide range of scenarios including workplace fatigue mitigation, meditation enhancement, memory retention training, vision therapy, and neurorehabilitation after stroke or trauma.

Various accessories may be provided with apparatus 100, such as a carrying case, removable hygiene liners, firmware programming cables, or supplemental data visualization tools for health practitioners. In certain use cases, the apparatus 100 may be integrated into broader therapeutic systems including synchronized breath control, auditory cueing, or tactile feedback via secondary wearables such as wristbands or chest straps.

Data collected and processed by apparatus 100 may be logged and optionally uploaded to cloud-based analytics platforms for longitudinal tracking, compliance verification, or comparative analysis against normative databases. The outer shell 100A may include customizable features such as adjustable straps, padding configurations, or aesthetic covers to support user preferences and encourage regular usage.

In multi-user environments such as clinics or research labs, apparatus 100 may include user authentication mechanisms such as RFID-based login or facial recognition, linked to secure user profiles stored locally or remotely. Emitter portions 101A-101E and the inner liner shell 100B may be color-coded or labeled to assist in correct placement and configuration, particularly when modular components are designed to be swappable or replaceable.

The apparatus 100 may include diagnostic functions that guide the user through setup validation steps, such as confirming contact integrity, verifying emitter responsiveness, or calibrating sensor baselines. The control system of the apparatus 100 may be programmable via a graphical user interface (GUI) accessible on a mobile device, computer, or touchscreen panel embedded in the helmet itself.

In some embodiments, the emitter portions 101A, 101B, 101C, 101D, and 101E may be permanently integrated into the structure of the neurostimulation apparatus 100 or may alternatively be configured as modular components that are removably attachable, allowing for replacement, customization, or maintenance. The apparatus 100 may further include a touch-sensitive interface comprising a display screen, which may be used to present therapy options, receive user input, display session status, or enable direct interaction with therapy settings without the need for a separate user device.

Referring now to FIG. 1A, exemplary configurations of energy emitter portals 102A, 102B, and 102C are illustrated, each configuration comprising one or more energy emitters 110 and one or more sensors 111 arranged within a single localized portal structure, in accordance with various embodiments of the present invention. These portal configurations (102A, 102B, and 102C) may be implemented in a head-mounted neurostimulation apparatus (100), such as the one illustrated and described in FIG. 1, where a plurality of such portals 102 are integrated across emitter portions 101A through 101E.

Portal 102A illustrates a configuration in which a central emitter 110 may be surrounded by a circular array of six peripheral sensors 111. This arrangement allows for localized emission of energy while simultaneously capturing a ring of sensor data from multiple angles around the emitted region. Such a configuration may be well-suited for applications in which real-time spatial feedback is used to monitor biological responses from different peripheries adjacent to the stimulation site. In some embodiments, emitter 110 in this configuration may comprise a near-infrared LED diode, while sensors 111 may comprise photodetectors, thermistors, or EEG electrodes.

Portal 102B presents an alternate configuration in which four emitters 110 and four sensors 111 are arranged in an interleaved alternating pattern. This symmetrical distribution allows balanced stimulation and sensing across a single compact region of the scalp. This layout facilitates applications in which both the emission and sensing must be synchronized in time and uniformly distributed in space. For example, the system may simultaneously monitor EEG fluctuations while delivering pulsed energy to modulate brainwave activity.

Portal 102C shows a dual-component configuration comprising one emitter 110 and one sensor 111 arranged asymmetrically. This configuration supports directional targeting, such as when stimulation is desired on one side of a neural region while measurement is collected from another. The spacing between the emitter 110 and sensor 111 in this arrangement may be variable, thereby allowing angular or lateral separation to be leveraged in therapeutic strategies such as hemispheric balancing or lateralized stimulation protocols.

In some embodiments, the emitter 110 of portal 102A may operate in a pulsed wave mode at a frequency of 10 Hz to stimulate alpha wave activity, while the surrounding sensors 111 detect regional thermal response and electrophysiological signals to characterize user relaxation states. The circular symmetry in this configuration allows spatial averaging and detection of anomalous asymmetries.

In another embodiment, portal 102B may be integrated into emitter portion 101A, which is aligned with the user's prefrontal cortex. Here, emitters 110 may emit red light at 660 nm while sensors 111 detect pulse-wave velocity or oxygen saturation, facilitating cognitive enhancement protocols where cerebral blood flow modulation is a goal.

Portal 102C, with its asymmetrical configuration, may be positioned in emitter portion 101C to support lateralized stimulation targeting the right parietal cortex. This configuration may be effective in scenarios involving hemispheric activation asymmetry, such as in attention modulation therapies or stroke rehabilitation tasks focused on specific lobes.

Each of the configurations 102A, 102B, and 102C may be fabricated on a common substrate using micromachining or printed flexible circuit technologies. Emitters 110 and sensors 111 may be bonded onto these substrates using conductive adhesives or micro-soldering techniques, and the entire portal assembly may be encapsulated in a transparent biocompatible polymer to isolate skin contact points while maintaining optical coupling.

In some embodiments, emitter 110 in portal 102A may be configured to emit modulated near-infrared energy in the 810 nm range, where penetration depth is optimized for cortical activation, while sensors 111 operate as short-circuit current detectors that measure reflected photon density, indicating local hemoglobin response.

Portal 102B's interleaved emitter-sensor array may be operated in a sequenced time-multiplexed scheme, where each emitter 110 activates sequentially while adjacent sensors 111 simultaneously capture differential response data. This method allows the controller to model energy diffusion patterns and dynamically tune emission parameters for optimal user response.

In portal 102C, the physical separation of emitter 110 and sensor 111 allows for time-delayed feedback measurements, such as detecting metabolic responses or neural delays after energy application. This may particularly be beneficial for research-driven applications where signal latency and propagation characteristics are to be studied.

In some embodiments, sensors 111 in portals 102A-102C may include surface-mounted biopotential electrodes, each configured to record electrophysiological signals with minimal impedance. These sensors may be fabricated using Ag/AgCl coatings and may optionally include gel-reservoirs or dry-contact surfaces depending on use conditions.

Alternatively, sensors 111 may be configured as optical sensors tuned for near-infrared reflectance, allowing simultaneous photoplethysmographic analysis during light therapy sessions. Such integration allows real-time monitoring of tissue perfusion and vascular tone.

Emitters 110, in various embodiments, may include broadband light sources capable of being tuned to emit in red, blue, green, or infrared wavelengths depending on the therapy protocol selected by the system controller or the user interface.

The use of circular configurations in portal 102A enhances thermal dissipation and allows uniform cooling, especially when continuous or high-intensity stimulation is applied. Materials such as copper-backed PCB or aluminum nitride may be employed to manage heat.

Portal 102B's arrangement may be advantageous in layered sensing-emission designs where vertical stacking is impractical. The side-by-side configuration allows planar integration without increasing device height, thus preserving ergonomic comfort for the user. In some embodiments, portal 102C may include sensor 111 configured as a motion detector or accelerometer embedded within the substrate. This setup enables correlating energy stimulation to postural or behavioral changes detected via motion.

Each portal configuration 102A, 102B, and 102C may further include conductive shielding or isolation layers to prevent crosstalk between emitter 110 and sensor 111. This may particularly be relevant in configurations like 102B where inter-component proximity is minimized. Electrical routing from the emitters 110 and sensors 111 may be handled via multilayer flexible circuitry extending from each portal to the central controller, where digitization, signal processing, and closed-loop algorithm execution is performed.

The selection of portal configuration for a given emitter portion (e.g., 101A or 101E) may be based on clinical objectives, such as mood regulation, sleep induction, alertness modulation, or motor recovery support. Each configuration allows tailored spatial and functional engagement with the underlying neural substrate.

Multiple types of these portals may coexist within a single helmet, allowing hybrid operation where different regions of the brain receive stimulation via portals configured as 102A, while others are monitored via 102C. In some embodiments, emitter 110 in portal 102A may be configured to modulate its output frequency based on signal coherence detected by surrounding sensors 111. When coherence increases beyond a threshold, the controller may attenuate stimulation or transition to a different protocol.

The mechanical assembly of portals 102A-102C may include soft elastomeric gaskets or memory foam to maintain consistent contact pressure across irregular scalp surfaces, thereby improving both emission efficiency and sensor signal quality. Portals may be color-coded, illuminated with diagnostic LEDs, or equipped with tactile markings to facilitate manual alignment or maintenance procedures by the user or technician.

The dimensions of each portal may range from 5 mm to 20 mm in diameter, depending on the density of emitter 110 and sensor 111 components and the anatomical target zone. The casing may be circular, hexagonal, or polygonal depending on the packing arrangement required. In some embodiments, sensors 111 in configuration 102B may be programmed to detect sudden voltage spikes indicative of epileptiform activity. The system may use this detection to automatically suppress emitter 110 output and alert the user or healthcare provider.

Portal 102C may be incorporated in regions like the temple or mastoid area, where it is preferable to isolate sensing from stimulation due to user sensitivity or contraindications for energy delivery. Emitters 110 may support pulse width modulation (PWM) or amplitude modulation (AM) schemes, with frequency ranges from 0.5 Hz to 100 Hz for synchronization with specific brainwave patterns. Sensors 111 may concurrently monitor entrainment metrics.

The substrate material housing emitter 110 and sensor 111 in each portal configuration may include polyimide, PET, or thermoplastic silicone, depending on flexibility requirements and thermal tolerance. Each of the portals 102A-102C may be indexed or registered in software as an addressable node, allowing the controller to activate, monitor, or calibrate each independently based on user profile or therapy session history.

In a system-wide architecture, a combination of 102A, 102B, and 102C portal types may be used to implement a spatial therapy map, where portal types are assigned based on regional sensitivity, cortical density, or therapy responsiveness trends. Additional variations of the configurations shown in FIG. 1A may include dual-emitter single-sensor layouts, quad-sensor orthogonal arrangements, or concentric ring emitters with central sensors, depending on design evolution.

Firmware governing the operation of emitter 110 and sensor 111 in each portal may include safety cutoffs, data smoothing filters, and real-time diagnostics to support uninterrupted and reliable operation. The combination of these portals within a wearable neurostimulation system enables adaptable, responsive, and location-specific modulation of brain activity for both wellness and clinical applications, forming the basis for diverse embodiments described herein.

Referring now to FIG. 1B, an exemplary head-mounted neurostimulation apparatus 100 is illustrated. The apparatus 100 may comprise a unitary energy emitter portion 101 (instead of multiple energy portions 101A-101E) configured with a plurality of integrated energy emitter portals 102, wherein the emitter portion 101 functions as a singular emitter array substrate for delivering stimulation across multiple regions of the user's head. Unlike modular arrangements where emitter portions are segregated into distinct physical zones, the embodiment shown in FIG. 1B presents a cohesive and uninterrupted architecture that promotes structural uniformity and design simplification.

The unitary energy emitter portion 101 may be fabricated using a continuous thermoplastic elastomer or a flexible polymer composite material that offers conformal coverage across the user's cranial contour. This configuration allows for better distribution of contact pressure, improved fit across various head sizes, and reduced complexity in emitter alignment. In some embodiments, materials such as polyimide, TPU, or silicone may be laminated with conductive pathways to support embedded electronics.

The integrated energy emitter portals 102 may be strategically embedded across the surface of the emitter portion 101. These portals 102 may include a combination of light emitters, electrodes, temperature sensors, and other bio-responsive components. The uniform grid layout of energy emitter portals 102 enables the apparatus 100 to stimulate multiple brain regions simultaneously or sequentially based on selected therapy profiles.

In some embodiments, each energy (e.g., light) emitter portal 102 may be equipped with an LED diode configured to emit narrow-band light in the near-infrared (NIR) range, ideal for photobiomodulation treatments targeting deeper neural structures. The distribution of portals 102 across the emitter portion 101 is designed to maximize surface area coverage without overlapping energy fields.

The continuous shell configuration of emitter portion 101 allows the emitters (102) to follow a radial or concentric pattern originating from a central point near the crown of the head. This pattern may be optimized for uniformity in light distribution or synchronized phase emission for hemispheric entrainment. This layout can also be adapted for circular, elliptical, or skull-conformal grids depending on therapeutic requirements.

In some implementations, the unitary emitter portion 101 may be contoured to provide increased density of energy emitter portals 102 in high-function neural zones such as the prefrontal cortex or visual cortex regions, while maintaining lower density in less-sensitive or non-therapeutic areas. This non-linear distribution permits energy conservation and targeted impact.

The structure of emitter portion 101 may also incorporate embedded waveguides or fiber optic filaments to channel light from centralized emitters to distant regions within the shell. This technique can enable use of fewer high-efficiency emitters while maintaining comprehensive stimulation coverage across the scalp.

The apparatus 100 may also include dedicated sensors 103 that may be integrated directly into the same substrate as energy emitter portals 102 or positioned at selected high-value sites across the emitter portion 101. These sensors 103 may comprise EEG electrodes, photodetectors, or biometric modules configured to capture neural or physiological responses in real time.

The combination of uniform emitter placement and embedded sensors allows the apparatus 100 to operate in a closed-loop feedback system where sensor readings directly influence stimulation output. For example, reduced alpha activity in a frontal region may automatically trigger adjustment in light frequency or pulse modulation for the corresponding portals 102.

The unitary design of emitter portion 101 further allows a more compact and self-contained electronics assembly. Control circuitry, memory components, power delivery systems, and communication modules may all be distributed within or along the perimeter of the shell, removing the need for external mounting or compartmentalization.

In some embodiments, the emitter portion 101 may be produced through additive manufacturing methods, such as 3D printing or molded polymer casting. These techniques allow for precise embedding of energy emitter portals 102 and sensors 103 within a single fabrication process, enhancing reliability and reducing manufacturing costs.

The interface between the emitter portion 101 and the outer shell 100A may be secured using adhesive bonding, friction-fit tabs, or locking clips. This attachment may be semi-permanent or serviceable, allowing replacement of the emitter portion 101 when required due to wear, upgrades, or hygiene protocols.

In alternate embodiments, the emitter portion 101 may be configured to support multiple types of therapy through a programmable control unit. For example, certain sets of energy emitter portals 102 may be activated for deep relaxation sessions using pulsed low-frequency light, while others may be reserved for cognitive engagement therapies using high-frequency stimulation.

The energy emitter portals 102 may be arranged in rows and columns or in an organic pattern designed to align with EEG scalp zones (e.g., 10-20 international system locations). This alignment supports research-grade neuromodulation therapies and allows consistent targeting of neural circuits across sessions.

The emitter portion 101 may also support differential control zones, wherein separate groups of emitter portals 102 are controlled independently. These control zones may be predefined or user-configurable through a graphical user interface, allowing real-time selection and modification of active regions during a session.

Sensors 103 may also provide diagnostic capabilities, such as verifying contact integrity, detecting dry skin conditions, or monitoring thermal load near each active portal 102. Such feedback helps maintain optimal stimulation conditions and enhances session safety.

The surface of the emitter portion 101 may include ergonomic contours or recessed channels that promote airflow and reduce sweat accumulation. These design features also improve user comfort during extended sessions and reduce the risk of slippage or poor contact.

In certain embodiments, the emitter portion 101 may be lined with antimicrobial or hypoallergenic coatings to support medical and personal use across diverse populations. Replaceable liners or disposable hygiene covers may also be employed without interfering with the functionality of portals 102 or sensors 103.

The outer shell 100A may serve as a protective housing for the emitter portion 101 and its components while also offering aesthetic customization. The emitter portion 101 may remain discreetly hidden while allowing maintenance access through openings, removable panels, or user tools.

Portals 102 embedded in the emitter portion 101 may support a variety of emission waveforms including sinusoidal, triangular, burst, and pseudo-random patterns. These waveform options may be selected based on stimulation objectives such as enhancing neuroplasticity, reinforcing memory encoding, or dampening anxiety-associated rhythms.

Each energy emitter portal 102 may be uniquely addressable through a digital multiplexing network integrated within the emitter portion 101. This allows for complex sequences and patterns of activation across the surface of the emitter portion 101 without requiring individual wiring for each emitter (102).

In addition to emitting and sensing capabilities, some portals 102 may incorporate microcontrollers or field programmable gate arrays (FPGAs) to support localized processing, energy modulation, or communication relay functions, thus decentralizing the control logic.

The uniform design of the emitter portion or shell 101 simplifies packaging and transport, particularly when the apparatus 100 is used in professional settings such as clinics, laboratories, or distributed home-based care environments. The reduced part count and integration reduce the likelihood of assembly errors.

Thermal modeling of the emitter portion 101 during use may be performed to optimize portal placement, minimize heat buildup, and support safe operation during long therapy sessions. Active cooling systems may be integrated if required, though passive thermal design is generally sufficient. In some embodiments, the emitter portion 101 may also incorporate light-diffusing layers above the emitters in portals 102. These diffusion layers scatter emitted light across a wider area, improving tissue coverage and reducing point intensity, thus promoting more even stimulation.

The unitary construction allows software-based zoning to virtually partition the surface of the emitter portion 101 into therapy areas. For example, the system may define frontal, parietal, temporal, and occipital zones without requiring physical separation. Power delivery to the portals 102 may be managed through flexible bus lines embedded in the emitter portion 101 substrate. These buses may include redundancy paths to accommodate wear or microfractures that may develop over prolonged use.

The uniform layout also permits testing and calibration during manufacturing or maintenance via automated probes that contact specific portals 102 to verify alignment, intensity, and electrical connectivity. In some applications, portals 102 may be programmed to vary light emission parameters across different axes. For example, rows may emit in red light while columns emit near-infrared, enabling cross-modality studies and adaptive therapy patterns.

The integration of the sensors 103 into the emitter portion 101 may allow machine learning models to track user performance across sessions. The system may build personalized stimulation profiles by correlating sensor data with user outcomes.

The head-mounted apparatus 100, with unitary emitter portion 101, may be designed to support adjunct systems, such as auditory feedback via bone conduction speakers, vibration motors, or thermal cues, all of which can be positioned or embedded along with portals 102.

Electrical connectors and user interface modules may be mounted near an accessible portion of the emitter portion 101, routing through the outer shell 100A to support wired programming, diagnostics, and device management. Each energy emitter portal 102 may be overlaid with a semipermeable membrane or film that maintains transparency to the emitted wavelength while protecting the underlying componentry from moisture or debris.

The uniform inner shell or emitter portion 101 construction may be well-suited for autonomous use cases, where minimal user input is required. Pre-programmed sequences, automatically triggered responses, and adaptive routines may all be managed by onboard firmware linked to sensor 103 feedback.

The system's firmware may support environmental awareness by adjusting stimulation based on ambient lighting, user location (indoors vs. outdoors), or time of day, enhancing circadian alignment or therapy consistency.

In educational or training contexts, the apparatus 100 may be used to support focus retention, memory encoding, or language acquisition through neuromodulation routines delivered via the energy emitter portals 102 of emitter portion 101.

The architecture of emitter portion 101 permits integration with VR or AR systems by aligning stimulation with immersive visual or auditory stimuli, supporting cross-sensory reinforcement in neuroplastic learning systems. The emitter portion 101 may optionally support expansion ports or magnetic docks that allow additional components, such as goggle attachments or chin straps with embedded sensors, to interface seamlessly with the existing electronics.

User access panels or contact markers may be molded into the emitter portion 101 to facilitate alignment, fitting, or repositioning. These may be indexed to anatomical landmarks for repeatable and consistent placement.

The use of a single shell structure (101) enables a more robust mechanical design that resists torsion, bending, and deformation, thereby preserving portal 102 alignment even under user motion or prolonged usage. In remote healthcare or teletherapy settings, the apparatus 100 with emitter portion 101 may be remotely configured, monitored, and updated via cloud-connected interfaces that rely on real-time data streamed from sensors 103 and actuator logs.

The unitary emitter portion 101 architecture opens further avenues for AI-powered control, where onboard processors analyze sensor trends to dynamically optimize which portals 102 to activate, for how long, and with what waveform characteristics. The shell design promotes manufacturing efficiency, as a single injection-molded or printed component may serve multiple user types, simply by modifying software mappings, overlay configurations, or attachment kits.

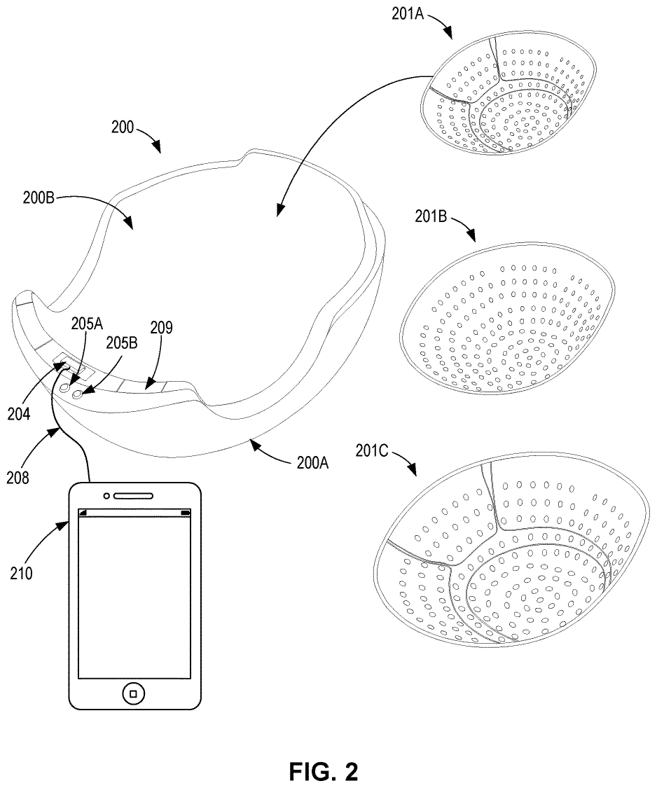

Referring now to FIG. 2, an exploded view of a head-mounted wearable apparatus 200 is illustrated. The apparatus 200 includes a plurality of interchangeable modular emission inserts 201A, 201B, and 201C configured for energy delivery within the apparatus 200. These inserts may be selectively installed into an inner cavity 200B that defines a structural enclosure for receiving and securing the selected emission insert. The design shown in FIG. 2 provides flexibility and adaptability in neurostimulation and therapeutic applications by allowing users or administrators to replace the inner stimulation layer according to session requirements, user physiology (e.g., head size or shape), or targeted functionality.

The apparatus 200 may be composed of a rigid or semi-rigid outer shell 200A and the inner cavity 200B shaped to conform to the user's cranial profile. The outer shell 200A may be fabricated using materials such as reinforced thermoplastics, medical-grade polymers, or layered composites that offer strength, low weight, and impact resistance. The cavity 200B may be shaped to ergonomically accommodate the head and to securely hold any of the modular inserts 201A, 201B, or 201C, each of which includes one or more emitter elements.

The modular insert 201A may include multiple emitter portions defined by separate zones that can independently target specific neural regions of the user's scalp. Each zone may carry its own set of emitters and, optionally, sensors for measuring brainwave activity, temperature, pulse rate, or other bio-indicators. The configuration of 201A may reflect the design strategy shown in previous figures where modular segments (e.g., 101A-101E) correspond to localized cranial anatomy such as frontal, parietal, temporal, and occipital lobes.

Modular insert 201B, by contrast, is shown as a unitary emitter shell, representing a continuous substrate embedded with an array of emitters and, optionally, sensors, similar to the structure (101) described in FIG. 1B. This design simplifies manufacturing and user operation, as it provides homogeneous coverage across the inner surface of the apparatus 200. It can be particularly advantageous for users seeking consistent whole-head stimulation without needing to select or configure individual zones.

Insert 201C represents another variation with segmented zones similar to 201A but shaped differently, potentially optimized for a different head profile, therapy protocol, or user demographic. The system may allow switching between insert types based on session data, therapeutic feedback, or clinician recommendations. Each insert may include alignment features or magnetic couplers that facilitate secure and guided placement within the inner cavity 200B.

The apparatus 200 may include a connection port 204 integrated into the outer shell 200A, through which data and power can be transferred to and from the insert inside. The port 204 may be configured as a USB-C connector, micro-USB, or proprietary magnetic data interface. It supports secure tethering to an external user device 210 via a wired connection 208 to avoid RF-based wireless communication during active use, thereby minimizing exposure to electromagnetic radiation.

Two external control buttons 205A and 205B are shown adjacent to the connection port 204. These buttons may be mechanical, capacitive touch, or pressure-sensitive inputs. Button 205A may serve as a master power toggle, while button 205B may activate specific operational modes such as therapy program selection, session start/pause, or data transfer initiation. The buttons may also serve for user authentication or pairing procedures.

In certain embodiments, modular inserts 201A, 201B, and 201C may contain their own embedded microcontroller, which communicates with the main control circuitry of the apparatus 200 via the interface at port 204. These microcontrollers may manage emitter timing, frequency adjustment, and sensor data preprocessing.

User device 210 is illustrated as a touchscreen smartphone, tablet, or dedicated therapy management device. It may run a software application that interfaces with the apparatus 200 to configure therapy settings, monitor session performance, and display analytics derived from sensor feedback. Connection may be established through wired cable 208, which may be sheathed for durability and interference reduction.

The user device 210 may offer a graphical user interface (GUI) through which a user or clinician selects therapy modes, such as relaxation, attention focus, memory training, or sleep induction. Each mode may correspond to specific emission parameters, including frequency, pulse width, intensity, and duty cycle across different regions of the modular insert.

In some embodiments, device 210 may detect the type of insert installed in the apparatus 200 through electrical identification chips embedded in the inserts 201A-201C. This detection enables the software to auto-configure therapy parameters and disable incompatible modes, providing compatibility with the insert's capabilities.

User device 210 may also serve to log historical data received from sensors integrated within the insert. These logs may include biofeedback trends, compliance metrics, and cumulative usage, all of which may be transmitted to a secure cloud server for remote analysis or clinical review.

Inserts 201A through 201C may be designed to allow periodic replacement or upgrade. For example, a user may begin with a basic insert 201B optimized for general wellness and later switch to insert 201A for more targeted neurotherapy. Each insert may be serialized and tracked via app-integrated registration systems.

The design of apparatus 200 may be modular not only in terms of its inserts but also in terms of compatibility with various outer shells 200A. These shells may come in different sizes, shapes, or materials for different market segments or environmental use cases, such as fitness, meditation, or medical rehabilitation.

Insert 201B, having a unitary shell architecture, may be easier to sterilize and maintain, especially for clinical use where sanitation and standardization are important. Conversely, segmented inserts such as 201A and 201C may offer finer therapeutic control but involve more intricate manufacturing and alignment.

Each modular insert may include multiple types of energy emitter technologies. For example, 201A may feature red and infrared light emitters, whereas 201C may incorporate a combination of light, thermal, and vibrotactile emitters. This adaptability expands the apparatus 200's applications beyond light therapy to multimodal neuromodulation.

The mechanical coupling between the insert (201A-201C) and the inner cavity 200B may utilize magnets, clips, grooves, or pressure-sensitive adhesives. Additionally, flexible printed circuits may bridge the insert (201A-201C) to the internal wiring leading to the port 204, and buttons 205A-205B, maintaining a secure electrical interface.

Temperature sensors, motion detectors, and electrodermal activity sensors may be distributed throughout each insert (201A-201C), allowing real-time adjustment of therapy intensity and feedback-based customization. These features are managed via controller circuitry integrated either in the insert itself or in a processing unit within a housing 209 in the outer shell 200A.

The control buttons 205A and 205B may be supplemented with voice control, gesture-based inputs, or companion accessories, such as foot pedals or wristband remotes. These alternatives provide accessibility to users with motor limitations or cognitive impairments.

User device 210 may further be used to access training modules, user instructions, guided meditation sessions, or cognitive exercises that complement the stimulation therapy. These modules may be synchronized with active stimulation to provide integrative therapy. Session duration, stimulus sequencing, and emitter zone mapping may be configured via templates on the user device 210. These templates may be predefined by medical professionals, auto-generated through AI algorithms, or manually adjusted by experienced users.

The wired interface 208 between the user device 210 and the apparatus 200 may also support charging functionality, where internal batteries in the outer shell 200A or insert components are replenished when connected. In some embodiments, modular inserts 201A through 201C may include memory storage to locally save session settings or usage statistics, allowing continuity even if the user device 210 is changed, updated, or temporarily unavailable.

Multiple inserts may be shipped with the apparatus 200, offering a starter set of options that the user can experiment with to determine the most effective stimulation strategy. The system may record and recommend the most beneficial insert based on user-reported outcomes and sensor data. In embodiments where data privacy is critical, the user device 210 and apparatus 200 may incorporate encryption protocols, biometric authentication, or secure boot procedures to restrict unauthorized access.

The modular nature of inserts 201A-201C facilitates localized repair or diagnostics. If one emitter or sensor malfunctions, the corresponding zone or module can be isolated and serviced without replacing the entire apparatus. Insert 201A may also be optimized for clinical trials or research studies, where different groups of participants are exposed to distinct stimulation protocols. Each group can be assigned a specific insert version, allowing control and reproducibility.

Inserts may support temperature-controlled stimulation where embedded thermoelectric elements are activated based on biofeedback. Such a feature enables warmth-based relaxation therapy or cooling for inflammation control. Modular inserts may also include structural layers that dampen vibration or distribute pressure uniformly, improving user comfort and long-term wearability. These layers may be integrated using multi-material molding or lamination techniques.

Inserts 201A-201C may optionally incorporate stretchable electronics, allowing the stimulation surface to expand or contract to match the user's unique cranial geometry. This property enhances contact fidelity across diverse skull shapes. In addition to the user device 210, the apparatus 200 may also connect to clinical consoles, research instrumentation, or caregiver dashboards for supervision, configuration, and documentation.

Software controlling the apparatus 200 may feature usage analytics, such as total stimulation time, regional energy delivery distribution, session frequency, and compliance summaries, all collected through sensors and portal activation records. Depending on therapy objectives, inserts may be configured to deliver synchronized stimulation with external stimuli such as audiovisual content, user movements, or environmental cues (e.g., light cycles, soundtracks).

In some embodiments, the user device 210 may be operatively connected to the neurostimulation apparatus 200 and configured to provide auxiliary processing capabilities that support or augment the functioning of the apparatus 200. The user device 210, which may include a smartphone, tablet, or computing terminal, may execute software applications that perform various functions such as AI-based analysis of physiological data received from the apparatus, interpretation of user responses to on-screen questionnaires, and real-time or scheduled therapy session activation. Additionally, the user device 210 may facilitate on-demand modification of therapy parameters including stimulation frequency, emitter selection, intensity, duration, or pattern type. In certain implementations, the user device 210 may further provide access to historical session data, visualization of user progress, remote configuration updates, or communication with caregivers and cloud servers, thereby enhancing the personalization, control, and adaptability of the neurostimulation system.

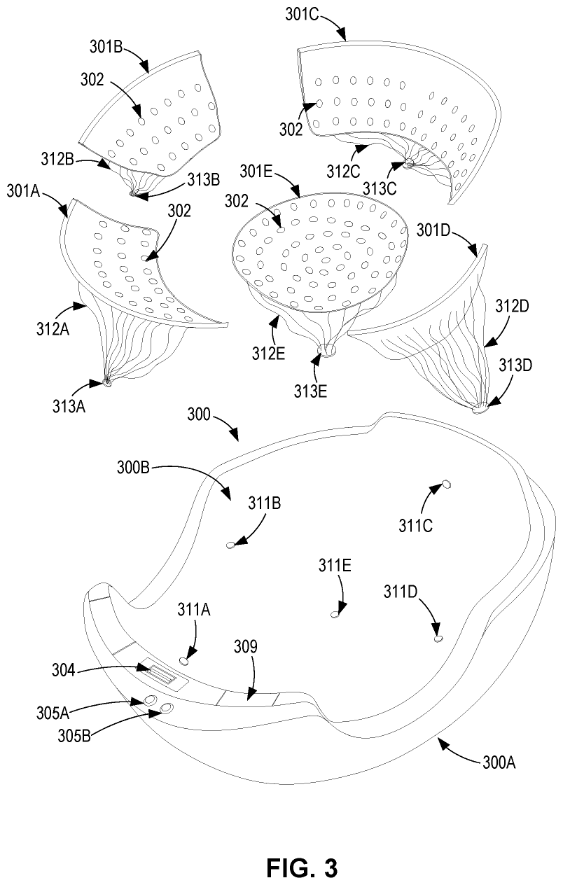

Referring now to FIG. 3, an exploded view of a modular head-mounted wearable apparatus 300 is illustrated. The apparatus 300 includes a plurality of removable emitter portions 301A, 301B, 301C, 301D, and 301E, each having a respective array of energy emitter portals 302. These emitter portions are configured to be removably inserted into an inner cavity 300B of an outer shell 300A. The configuration of FIG. 3 enables the apparatus 300 to be flexibly customized, serviced, or adapted for different therapeutic uses by swapping in different emitter portions.

The outer shell 300A forms the rigid or semi-rigid framework of the apparatus 300 and may be constructed using injection-molded polymers, carbon-fiber reinforced materials, or other medical-grade composites. The inner cavity 300B may be dimensioned and shaped to receive and retain the removable emitter portions 301A-301E, providing physical support, electrical contact, and thermal management features. The cavity 300B may include attachment grooves or slots to support positional alignment.

Each emitter portion 301A through 301E may be designed to target a specific region of the user's head. For example, emitter portion 301A may be aligned with the user's left temporal region, 301B with the left frontal region, 301C with the crown or vertex, 301D with the right parietal region, and 301E with the occipital region. These emitter portions may be pre-shaped and labeled for quick identification and guided installation.

Emitter portals 302 are distributed over each of the removable emitter portions 301A-301E in a predefined array. Each portal 302 may include an energy emitter configured to deliver light, sound, electrical, or other forms of stimulus. These portals 302 may be independently addressable by the apparatus 300, allowing selective activation based on user profiles, session parameters, or real-time biofeedback.

Each emitter portion 301A-301E may further be configured with a respective set of flexible links 312A-312E, which connect the emitter portals 302 to the main processing and energy generation components housed in housing 309 within the outer shell 300A. These links 312A-312E may comprise electrical wires, signal buses, or optical fibers depending on the transmission modality used for energy or data.

In embodiments where the links 312A-312E comprise electrical wires, these may carry voltage-controlled signals from the controller in housing 309 to the emitter portals 302. Alternatively, in embodiments where the links comprise fiber optics, light generated by diodes within housing 309 is guided to the emitter portals 302, enabling remote energy sourcing and reducing heat generation near the user's scalp.

The removable emitter portions 301A through 301E each terminate in first connectors 313A-313E, respectively. These connectors are designed to engage with matching second connectors 311A-311E embedded within the inner cavity 300B. The connectors may use mechanical pins, magnetic contacts, or optical couplers depending on the nature of the links and portals being used.

Connectors 313A-313E and 311A-311E may feature keying mechanisms to prevent incorrect installation. For example, connector 313A may be shaped uniquely to fit only into connector 311A. These keyed designs facilitate error-free assembly and enhance safety during use.

The modular nature of emitter portions 301A-301E allows for easy replacement and cleaning. Users may swap out one or more emitter portions to focus stimulation on new regions, replace defective parts, or adjust fit. Each emitter portion may also be made in different sizes or flexibilities to accommodate head shape variations.

The housing 309 embedded in the outer shell 300A contains the central control system, including a processor, memory modules, driver circuits for energy generation, and possibly an artificial intelligence (AI) engine for adaptive therapy management. It may also contain thermal sensors, power regulation circuits, and diagnostic tools to maintain consistent operational performance.

The apparatus 300 may include an external connection port 304 disposed on the exterior of the outer shell 300A. The connection port 304 may allow for data transfer, firmware updates, wired control by a user device, or charging of an internal battery system. The port 304 may comprise a USB-C, micro-USB, or magnetic charging interface.

Two command buttons 305A and 305B are shown adjacent to the port 304. These buttons may be used for local control of the apparatus 300, such as powering on or off, cycling through therapy modes, or toggling between manual and automated control states. Buttons 305A and 305B may be implemented as capacitive touch sensors, mechanical switches, or low-profile membrane controls.

The shell 300A may also include passive ventilation ducts or conductive layers to dissipate heat generated by the emitters. It may be coated with an antimicrobial or moisture-wicking material to enhance hygiene and comfort during extended use.

Each of the emitter portions 301A through 301E may be manufactured using flexible substrates such as silicone or TPU (thermoplastic polyurethane) to allow contouring around the user's head. They may be layered with insulative and protective coatings to preserve performance over time. In some embodiments, one or more emitter portions may include embedded sensors, such as EEG electrodes or photoplethysmographic sensors, interspersed among the emitter portals 302. These sensors may be used for real-time feedback or bio-response analysis.

The system may support multi-modal therapy where different emitter portions deliver different forms of stimulation. For example, portion 301A may deliver pulsed red light, while portion 301D delivers transcranial alternating current stimulation (tACS), enabling a comprehensive neuromodulation protocol.

To facilitate installation, each emitter portion may include visual markers, embedded magnets, or audible feedback indicators to confirm proper alignment and seating. The system may run a self-check once all emitter portions are connected to verify electrical continuity and system readiness.

The links 312A-312E may be detachable at both ends, allowing users to replace only the cabling in case of damage or degradation. Reinforced strain relief components may be integrated at each connector interface to protect against mechanical stress during installation or movement.

The modular system illustrated in FIG. 3 is also suitable for clinical or research use, where each emitter portion may be loaded with session-specific firmware or configuration files stored in local memory chips. These files may be read by the controller in housing 309 to guide automated stimulation routines.

Users may store multiple emitter portion sets customized for different use cases, such as daytime stimulation, nighttime relaxation, post-injury rehabilitation, or cognitive training. The system may automatically log which portions were used during each session for compliance tracking. In extended-use scenarios, the controller in housing 309 may be programmed to switch between emitter portions 301A-301E in a time-scheduled manner, directing energy delivery in waves across different scalp zones.

Each portal 302 within the emitter portions 301A-301E may be programmable for individual intensity, frequency, pulse duration, and emission angle, controlled via digital instructions transmitted over the links 312A-312E. The inner cavity 300B of the outer shell 300A may be precision-molded to accommodate the curvature and connection profile of each emitter portion. Interior padding or pressure-distributing inserts may be provided to enhance comfort and promote reliable contact.

In some implementations, the system may be used in conjunction with real-time monitoring devices such as smart watches, mobile EEG headbands, or sleep trackers. Data from these external sources may be fed into the controller in housing 309 to refine stimulation protocols. Each connector interface, such as 311A and 313A, may be ruggedized using water-resistant seals, low-profile locking mechanisms, and gold-plated contacts to support high signal fidelity over many use cycles. Modular emitter systems such as that illustrated in FIG. 3 may also facilitate testing of experimental protocols, where only a single emitter portion is swapped or modified while the remaining structure of the apparatus 300 is held constant.

Custom therapy packages may be distributed in sets of emitter portions, where users receive a kit containing insert 301A for anxiety relief, insert 301C for sleep support, and insert 301E for cognitive enhancement. Each portion may be activated as needed.

The apparatus 300 may include firmware allowing the user or system to detect which emitter portions are installed and to validate their operational readiness prior to session initiation. The controller may disable or warn against use if incorrect combinations are detected. Advanced AI features within housing 309 may evaluate past session effectiveness and sensor feedback to suggest which emitter portions to install next, or how to adjust their stimulation schedules.

This modular configuration promotes longevity of the apparatus 300, as worn or outdated emitter portions may be replaced without discarding the entire unit. Environmental and economic benefits are achieved through reduced material waste and modular servicing.

The modular emitter architecture supports a wide array of user demographics, including children, elderly users, and individuals with atypical cranial morphology. Inserts may be tailored for head size, therapy goals, or sensitivity thresholds. Visual status indicators, such as color-changing LEDs or haptic feedback motors, may be embedded into each emitter portion to signal activation, mode change, or error conditions.

Portions 301A-301E may include RFID or NFC tags to support secure user-specific settings or to guide automated tracking for usage analysis, especially in institutional settings. The shell 300A may be produced in various sizes to match user head geometry, while the emitter portions 301A-301E may remain standard, enabling scalable manufacturing and inventory management.

The command buttons 305A and 305B may be integrated into touch-sensitive regions of the shell surface or accessible through a mobile application interface connected via a data cable or detachable controller unit. The system may support remote therapy planning, where clinicians configure stimulation maps for each emitter portion 301A through 301E and transmit the configuration to the apparatus 300 for automatic execution.

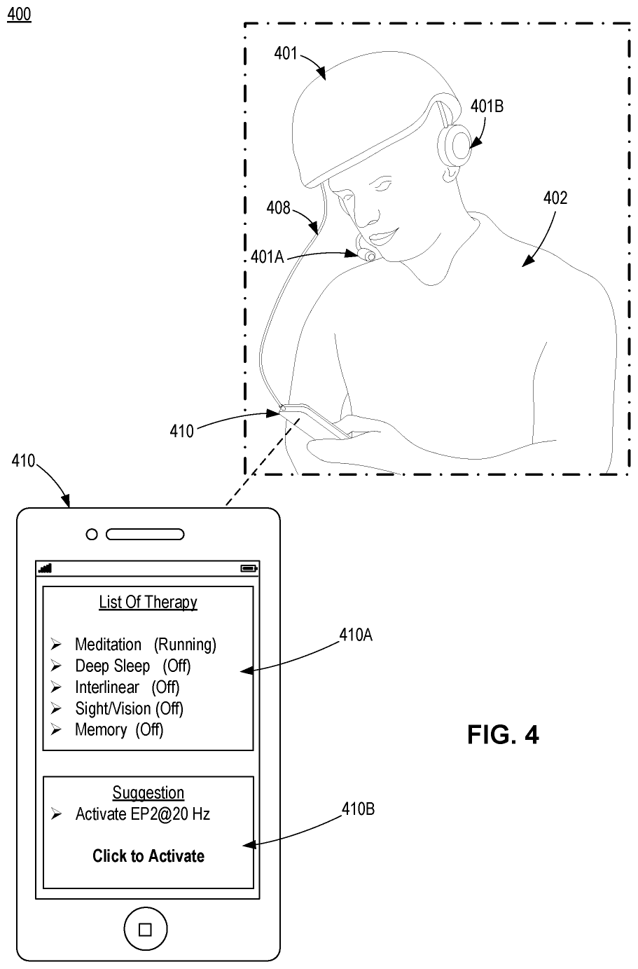

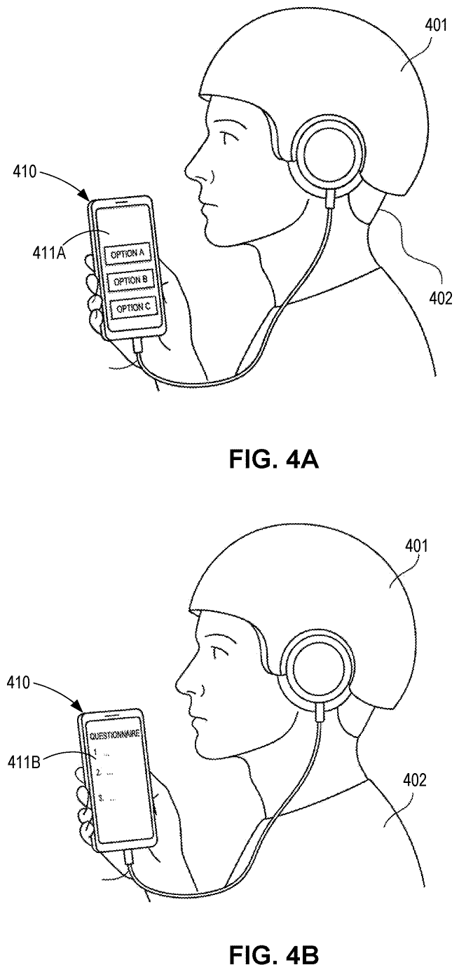

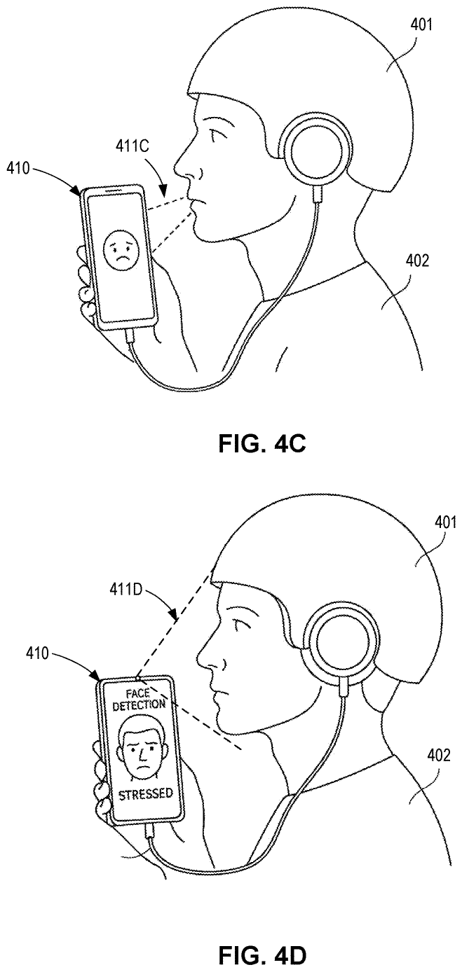

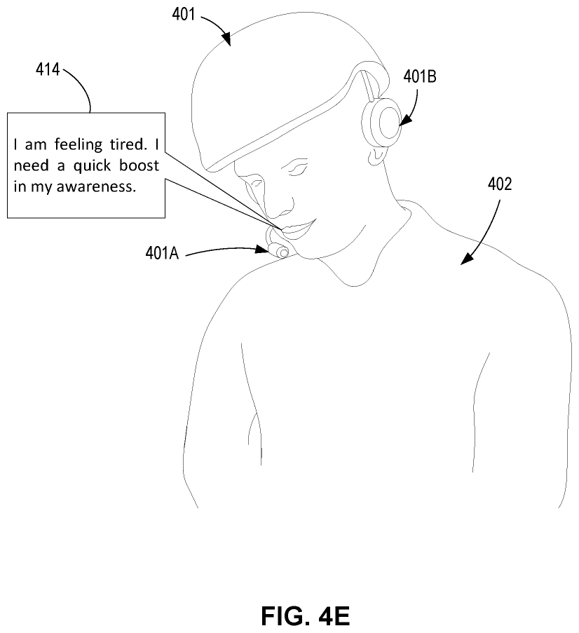

Referring now to FIG. 4, an exemplary system 400 is illustrated, wherein a head-mounted neurostimulation apparatus 401 is worn by a user 402 and connected to a user device 410 via a wired connection 408. The system 400 demonstrates interactive operation and real-time configuration of therapy protocols via the user device 410, which displays a plurality of therapy options 410A and associated stimulation parameters. This embodiment highlights how manual control of therapeutic routines can be facilitated by the user 402 in an offline mode, where electromagnetic interference is minimized through physical cabling rather than wireless transmission.

The neurostimulation apparatus 401 may be composed of a helmet-like outer shell designed to house internal energy emitter components, sensors, signal routing elements, and processing electronics. The apparatus 401 may be ergonomically configured to remain securely mounted on the head of the user 402 while delivering energy-based therapy via strategically placed emitter portals. These emitter portals may be positioned based on neurological mapping to stimulate specific cortical or subcortical brain regions.

In the embodiment of FIG. 4, the apparatus 401 may include a microphone 410A integrated on or near the front or side section of the shell. The microphone 410A may be configured to accept voice commands or queries from the user 402. These commands may activate or modify therapy protocols, request feedback, or trigger specific operational functions such as pausing, switching, or confirming therapy selections.

The apparatus 401 may further include an earphone or auditory transducer 401B positioned to deliver real-time audio cues or instructions to the user 402. This audio component may also be used to stream ambient soundscapes, therapy-related binaural tones, or calming music sequences based on the selected therapy type. In one example, during a deep sleep induction protocol, the earphone 401B may play low-frequency sound harmonics to promote alpha or delta wave entrainment.

The wired connection 408 between the apparatus 401 and the user device 410 may be configured for both data and power exchange. This approach avoids reliance on radiofrequency-based wireless systems, thereby reducing possible interference or biological sensitivity to such signals. The wired architecture also promotes faster response times, stable data throughput, and secure offline functionality.

The user device 410 may be any handheld or tabletop computing terminal, such as a smartphone, tablet, or custom-built neurostimulation controller. In some embodiments, the user device 410 may include a touch interface through which therapy options 410A can be selected or configured by user 402. The options may be displayed in a scrollable or categorized layout.

As illustrated, therapy options 410A may include “Meditation,” “Deep Sleep,” “Interlinear,” “Sight/Vision,” and “Memory.” Each of these entries represents a distinct neurological therapy program, tailored to activate specific neural pathways or brain regions. For example, the “Sight/Vision” option may initiate stimulation of the occipital lobe through emitter portals mapped to the rear portion of the helmet.

In one implementation, selecting the “Meditation” option activates a stimulation pattern targeted toward the prefrontal cortex, known to influence awareness, executive function, and emotional regulation. The stimulation waveform may involve low-frequency, modulated light or current pulses aligned with observed theta or alpha brain wave patterns.

The “Deep Sleep” therapy may operate by stimulating parietal and posterior cortical zones at frequencies between 0.5 and 4 Hz to induce delta brain wave patterns. The energy emitted may include near-infrared light pulses or low-amplitude alternating currents delivered through selected emitter portals. “Interlinear” therapy may correspond to a cognitive alignment or focus training protocol that modulates neural oscillation coherence between frontal and parietal regions. The system 400 may activate alternating emitter portals to stimulate both regions in synchrony with real-time phase adjustments based on feedback.

The “Sight/Vision” therapy option may activate emitter portals directed at the occipital cortex and related visual processing centers. Emitter patterns may involve pulsed near-infrared or red light at controlled intervals to promote blood flow, oxygenation, and neuroplasticity in the visual pathway circuits.

The “Memory” therapy program may activate mid-temporal and hippocampal-related zones, facilitated through emitter portals positioned laterally along the apparatus 401. Frequencies employed may range from 8 to 15 Hz to coincide with memory consolidation brain wave bands.

Upon selection of any therapy option 410A, the user device 410 may present an automated suggestion 410B, such as “Activate EP2@20 Hz,” where “EP2” refers to Emitter Portion 2 of the apparatus 401, comprising a distinct set of emitter portals. The system may identify the optimal emitter portion and stimulation frequency based on the chosen therapy.

The suggestion 410B may be generated based on pre-programmed therapy templates, machine learning models trained on population data, or previous session records associated with the user 402. Each suggestion may also include an explanation or informational tip regarding its purpose or target brain region.

The user device 410 may provide options to confirm, modify, or override the suggested parameter settings. The screen may further include adjustable sliders or dropdowns for selecting duration, intensity, waveform shape, emitter region, and synchronization modes.

Voice command capability via the microphone 410A allows the user 402 to operate the apparatus 401 without needing to handle the device 410 directly. The system may recognize commands such as “Start meditation,” “Switch to deep sleep,” “Increase frequency to 25 Hz,” or “Pause therapy.”

The earphone 401B may play prompts to guide the user through initial calibration, session start, therapy instructions, or relaxation techniques. In guided meditation, the voice output may include breathing cues or mantras synchronized with the stimulation.

Additional therapy options 410A that may be displayed include “Stress Reduction,” “Focus Enhancement,” “Neurorehabilitation,” “Balance and Coordination,” and “Language Learning.” Each option may correspond to a well-mapped set of brain targets and stimulation profiles.

For “Stress Reduction,” emitter portals along the prefrontal and insular cortex regions may be activated in slowly ramping waveforms, possibly using warm-spectrum light or gentle vibration pulses to reduce sympathetic nervous activity.

“Focus Enhancement” therapy may utilize emitter portals over the dorsolateral prefrontal cortex and anterior cingulate cortex, delivering short bursts of high-frequency light or electrical pulses to improve cognitive clarity and attentional control.

In “Neurorehabilitation” mode, stimulation parameters may vary based on clinical history. For stroke recovery, motor cortex regions may be stimulated while synchronizing with physical therapy movements, facilitated through accelerometer-based feedback in the apparatus 401.

“Balance and Coordination” therapy may engage sensorimotor networks through stimulation of parietal lobes and cerebellar zones, incorporating dynamic emitter sequencing to mirror proprioceptive pathways and vestibular cues.

“Language Learning” therapy may stimulate Broca's area and Wernicke's area bilaterally or alternately, leveraging frequencies known to support verbal memory formation and comprehension.

Each therapy selection 410A may launch a dynamic UI sequence on user device 410, including countdowns, visual feedback (e.g., brain maps showing active zones), and progress bars. The user may be able to track session duration, stimulation progress, and physiological changes.

The user device 410 may further display historical data, such as which therapy options were used, when, and how long. It may log physiological responses, alert the user to abnormalities, or adapt the therapy plan over time.

Therapy parameters suggested in 410B may be customized per user profile, which can store preferences, contraindications, response history, and biometric identifiers. The AI engine embedded in the apparatus 401 or the user device 410 may use this data to refine future sessions.

The system 400 may also support expert-mode operation, wherein advanced users or clinicians may directly access matrix control over emitter portions and individual portals, mapping custom stimulation patterns to defined brain coordinates. In some embodiments, each emitter portion (e.g., EP2) may be subdivided into zones, allowing zone-specific frequency or waveform tuning. The UI on device 410 may present a virtual representation of the user's head to graphically indicate active zones.

The wired connection 408 may include a braided or shielded cable with strain relief to support mobility during therapy. It may include a data-sync indicator LED or breakaway safety mechanism to protect against accidental detachment. The user device 410 may also serve as an access point for data export to cloud storage, where authorized medical professionals or researchers may review anonymized data for therapy optimization or efficacy evaluation.

In educational scenarios, students may use the apparatus 401 with learning-optimized stimulation settings during reading or studying, while the user device 410 provides time management tools and focus assessment based on stimulation pattern engagement. In sleep-related protocols, the apparatus 401 may remain in low-power mode overnight, cycling through programmed stimulation waves. The user device 410 may remain off or enter sleep mode to minimize distraction, resuming sync at session end.

Security features may be integrated, including user authentication via biometric or passcode on device 410, restricting access to certain therapies or settings for safety and compliance in multi-user or clinical contexts.

Customization capabilities may extend to therapy background visuals or sound themes, allowing user 402 to tailor their experience through the device 410 interface to increase engagement and adherence. The system 400 may support reward-based compliance, where consistent use or session completion unlocks new content or capabilities within the software on user device 410.

Referring now to FIGS. 4A-4D, various user interface modes associated with the head-mounted neurostimulation apparatus 401 are illustrated, wherein a user 402 operates a user device 410 to access distinct types of interactive functionality for therapy configuration and personalized stimulation delivery. The device 410 may be connected via a wired interface to the apparatus 401 to prevent electromagnetic signal emissions and to support secure, high-bandwidth communication. In each of these figures, the system dynamically adapts to user interaction styles, biometric inputs, and contextual parameters to present therapy recommendations or stimulation commands in accordance with various embodiments of the present invention.

In FIG. 4A, user device 410 displays a selectable therapy interface 411A comprising distinct therapy options. These therapy options 411A may include predefined categories such as Focus Boost, Memory Enhancement, Stress Reduction, Sleep Induction, Creativity Stimulation, and Vision Support. The options 411A are configured to trigger predefined energy stimulation profiles within the apparatus 401, involving activation of specific emitter portions and emitter portals aligned with target cortical or subcortical zones.

The therapy options 411A displayed in FIG. 4A may be generated based on stored user history, time of day, biometric patterns detected from embedded sensors, or recent user activity. For example, if a drop in heart rate variability or an increase in blink rate is detected, Stress Reduction or Focus Boost options may be elevated in priority within the options 411A set.

The options 411A in FIG. 4A may include dynamic therapy slots labeled “Adaptive Mode” or “Custom Sequence,” which trigger machine-learned protocols tailored to the user 402's neurological or behavioral profile. The system may retrieve these personalized protocols from local memory or through encrypted access to an external repository.

In some embodiments, selecting a therapy option in 411A such as Vision Support may activate emitter portals positioned to stimulate the occipital lobe and connected visual processing centers, delivering light at specific wavelengths (e.g., 660 nm red or 850 nm near-infrared) and at modulation frequencies optimized for neuroplastic adaptation.

In FIG. 4B, the user device 410 presents a questionnaire interface (411B). This interface may be designed to capture user feedback, preferences, or conditions prior to determining the ideal therapy routine. The questions 411B displayed within interface may vary daily or between sessions and may address topics such as emotional state, energy level, sleep quality, concentration ability, or recent physical exertion.

Example questions 411B shown in the interface may include: “How well did you sleep last night?”, “How focused do you feel right now?”, “Do you feel anxious or calm?”, “Have you experienced any headaches recently?”, or “Would you like to boost your creativity or concentration today?”

Based on responses to the questionnaire 411B, the controller of apparatus 401 or a processing module in device 410 may calculate weighted scores corresponding to therapy goals. The outcome may be used to rank and present therapy options in a personalized manner or directly trigger a recommended stimulation profile.

The questionnaire 411B may also be used for longitudinal tracking, where daily response trends are analyzed to assess user progress or shifts in cognitive and physiological patterns. Therapy suggestions may then evolve across sessions to reflect user trajectory.

The system may employ logic trees or Bayesian models to determine how responses for questions 411B influence therapy pathways. For example, a user who reports frequent distraction and low motivation over several days may be directed toward prefrontal-focused stimulation and arousal-enhancing light frequencies. In some embodiment, the stimulation energy delivered through the one or more energy emitter portals (or the apparatus 401) may be dynamically modulated in real time based on an AI analysis of at least one of: sensor input, user interaction, emotional state of the user (FIG. 4C-4D), and therapy progression.

FIG. 4C introduces an emotion detection interface based on user voice input 411C. In this interface, the user device 410 captures audio using an integrated or connected microphone to interpret emotional tonality, speech patterns, rhythm, or changes in vocal intensity.

Voice analysis may use AI algorithms such as convolutional neural networks (CNNs) or recurrent neural networks (RNNs) to identify emotional states such as fatigue, frustration, anxiety, contentment, or sadness. The detected emotional state may be used as an indirect input to therapy selection.

In a specific example, if the voice pattern analysis determines low energy and low variation in pitch, the system may interpret the user as fatigued and suggest a gentle Focus Enhancement or Alertness Boost session activating parietal and prefrontal emitter portals.

The voice-based detection interface may also be used as a verbal questionnaire platform, where the device 410 reads questions aloud and records the user's spoken responses. This hands-free mode may be beneficial for individuals with mobility constraints or during tasks like walking, meditation, or warm-up exercises.

The voice-detection system (e.g., 411C) may also detect stress through elevated pitch, faster speech, and breath irregularities. In such a scenario, a calm-down stimulation profile may be proposed, involving low-frequency emitter sequences and earphone-based playback of calming audio.

In FIG. 4D, the user device 410 presents a facial detection interface (411D), which uses the device's camera to analyze the user's facial expressions and infer emotional states. Detection features may include eyebrow tension, mouth curvature, gaze fixation, or blink rate.

Based on facial analysis (411D), the system may identify the user as “Stressed,” “Tired,” “Focused,” or “Uncertain.” These labels can feed into a decision engine that selects emitter profiles most relevant to the detected emotion. For example, stress detection may trigger stimulation over the prefrontal cortex and parietal lobes to aid in cognitive regulation.

The interface (411D) may also assess micro-expressions during a prompted response, such as reacting to displayed words or phrases, enabling deeper insight into subconscious emotional states or user discomfort with certain stimuli.

Facial detection 411D may be cross-referenced with user questionnaire responses (411B) to validate self-reported data or identify cognitive dissonance. If a user indicates calmness but facial expressions show tension, the system may offer options for recalibration or mindfulness therapies.

The facial detection 411D may be particularly useful in pediatric applications or with non-verbal users, allowing non-intrusive emotional state estimation for therapy planning. Combined with simplified options 411A, this mode enables responsive, nonverbal interaction with the system.

In various embodiments, two or more interfaces from FIGS. 4A-4D may be used in parallel to derive the most appropriate therapy pathway. For example, the facial detection interface (411D) and the questionnaire 411B may be used jointly to recommend personalized therapy sequences.

The combined use of voice input 411C and therapy options 411A may allow real-time command execution such as “Start memory boost” or “Begin vision clarity mode,” triggering specific emitter sequences in the apparatus 401.

Fusion of all four modes—411A, 411B, 411C, and 411D—may be employed in a comprehensive session pre-screening protocol, where the system cross-validates user inputs and biometric cues before initiating therapy.

Each interface, (411A through 411D), may feed input into a decision algorithm resident in the apparatus 401 or in the user device 410. This algorithm may generate emitter zone activation maps, pulse waveforms, frequency modulation schedules, and audio output selections.

Automated therapy options based on combined input modes may be displayed after input collection. These options may be ranked or color-coded by expected benefit, confidence level, or previous response success rate.

Stimulation profiles generated based on interfaces (411A-411D) may activate specific emitter portions of the apparatus 401, such as temporal lobes for memory therapy, occipital zones for vision stimulation, or frontal lobes for decision-making enhancement.

In some embodiments, the apparatus 401 may include internal sensors (e.g., EEG, temperature, or heart rate) that augment the detection capacity of interfaces (411B-411D), further refining therapy suggestions based on physiological alignment.

Therapy options shown in 411A may dynamically update in real time based on inputs from 411C and 411D. For example, if facial tension is suddenly observed, a pop-up suggestion for “Relaxation Mode” may appear with a prompt to initiate within seconds.

User interface designs for 411A-411D may be adapted for accessibility, with enlarged buttons, high-contrast modes, or audio instructions for users with visual impairments, cognitive disorders, or limited dexterity.

Each interface element may also be mirrored on a secondary caregiver or therapist dashboard to enable remote monitoring or intervention, particularly in clinical or assisted living environments.

In predictive modes, if a user repeatedly selects certain answers for questions 411B or expresses stress vocally 411C, the system may proactively schedule session reminders or suggest preventative therapies to avoid escalation. The voice and face detection modes, 411C and 411D, may also be used to gauge session effectiveness. For example, facial expression shifts from stress to calm at session end may be logged and scored as a successful outcome.

All interfaces, 411A through 411D, may be available as modules in the user application on device 410, allowing user 402 to select which interaction mode is most comfortable or effective during any given session. The apparatus 401 may internally log which input method led to each therapy initiation, enabling longitudinal analysis of interface effectiveness and user preferences.

Sensor fusion across visual (411D), audio (411C), and questionnaire (411B) modalities may reduce false-positive detections and support more nuanced and confident therapy mappings. Session summaries may reflect how data from interfaces 411A-411D converged to inform the selected emitter activation map, building user trust and offering insights into therapy personalization. Device 410 may allow exporting these session summaries in standardized formats (e.g., PDF, XML) for integration with medical records or third-party health platforms.

Each of the interfaces may also support onboarding walkthroughs, educating new users on what inputs are collected, how they influence therapy, and how to interact with the system effectively.

Ultimately, FIGS. 4A-4D illustrate a cohesive and adaptive framework by which user 402 may engage with the neurostimulation apparatus 401 using multiple complementary input methods to derive responsive and individualized therapy routines, where therapy output—including the activated emitter portions and stimulation parameters—is guided by multimodal contextual awareness.

In some embodiments of the present invention, the neurostimulation apparatus (401) may be configured to support an intelligent and adaptive questionnaire interface that guides users through a structured set of diagnostic or introspective prompts. The apparatus, either directly or via an operatively connected user device, may present a series of context-sensitive questions to the user, with the content and sequence of subsequent questions being dynamically updated based on prior responses, therapy history, ongoing sensor readings, or therapy progression (i.e., how well the therapy is proceeding). The question flow may be constructed using a decision-tree or AI-generated dialogue model, which evolves during a session to more accurately assess the user's neurological, emotional, or physiological state. This adaptive questioning framework enables a personalized diagnostic pathway for each session, reducing unnecessary input and increasing therapeutic relevance.

For example, In some embodiments, a user may begin a session by responding to a question such as “How well did you sleep last night?” with selectable options ranging from poor to excellent. Based on the response, the next question may either probe further into sleep patterns (e.g., “Did you wake up frequently?”) or pivot toward emotional state (e.g., “Do you feel anxious today?”). If the user reports high anxiety, the system may infer that a calming or parasympathetic stimulation routine is appropriate, and direct follow-up questions toward identifying specific symptoms like irritability, racing thoughts, or muscle tension. This logic-driven progression allows the therapy objective (e.g., relaxation, alertness, memory support) to be selected with higher specificity and contextual awareness.

The questionnaire system may be delivered via a touchscreen interface, voice recognition module, or external user device with graphical or audible interfaces. In some embodiments, the responses may also be inferred passively via emotion detection subsystems, such as tone analysis from a microphone or facial expression detection using camera-enabled user devices. The collected response data may be used not only to configure the current session but also stored to update the user's long-term therapy model for future personalization.

Further, in accordance with another embodiment, the neurostimulation apparatus may support the use of multiple user profiles, each corresponding to a different individual. This capability allows the same physical device to be shared among several users—such as members of a household, clients in a clinical setting, or participants in a wellness program—while preserving the personalization and continuity of each user's therapy regimen. Each profile may store individualized data including therapy preferences, stimulation history, sensor calibration baselines, AI-adapted models, emotional response logs, biometric identifiers, and progress metrics.

The apparatus may include a user identification module configured to recognize a user and retrieve the corresponding profile automatically. In some embodiments, identification may be performed using biometric inputs such as voice signature via a microphone, fingerprint or facial image via a connected user device, or pattern-based behavioral cues (e.g., therapy selection frequency, response timing, speech cadence). Upon identification, the apparatus may automatically retrieve that user's stored profile, which may reside in local memory, a paired user device, or a remote cloud-based storage server. In another embodiment, profile selection may occur manually via a drop-down interface or touch-based menu, allowing users to switch profiles on a shared terminal.

Once the profile is loaded, the apparatus may present the adaptive questionnaire interface with parameters tailored to that specific user, such as their preferred question format (e.g., multiple-choice, sliders, free-text), known areas of therapeutic focus (e.g., anxiety management, cognitive enhancement), or specific health conditions. For example, if a user has historically responded better to stimulation in the parietal region for attention-based therapy, or exhibited higher HRV stabilization after sleep support routines, such insights may guide the questioning path and eventual therapy configuration.

In some embodiments, the apparatus may prompt for user identity at the beginning of the session and switch the onboard model accordingly. In other configurations, the system may run in passive observation mode, allowing a user to initiate therapy without identifying themselves explicitly. After observing usage patterns or sensor data, the system may suggest profile matches based on known traits or behaviors, prompting the user to confirm or select the correct identity.

This user profile system may also support secure privacy and data separation across users. For example, in a multi-user family setting, children and parents may share the same apparatus but access entirely distinct therapy settings and histories, safeguarded by authentication tokens. In clinic-based deployments, therapists may manage multiple clients using a single device while maintaining HIPAA-compliant data segregation via profile encryption and session-based access logs.

Moreover, the adaptive questionnaire framework and profile-based therapy configuration may be interlinked with the AI engine in the apparatus or remote server. As more sessions accumulate for a given user profile, the AI model may refine its question selection, prediction accuracy, and stimulation personalization. For example, if a user tends to underreport stress verbally but exhibits physiological signals consistent with high sympathetic activity, the system may begin to insert stress-related questions at a higher priority in future sessions or recommend additional sensor probes to augment emotional detection.

Referring now to FIG. 4E, an exemplary head-mounted neurostimulation apparatus 401 is illustrated, configured for voice-based operation wherein a user 402 provides verbal input 414 through a microphone 401A integrated into the apparatus 401 to initiate, adjust, or terminate therapy protocols, in accordance with various embodiments of the present invention. Unlike embodiments shown in earlier figures where a separate user device facilitated the interaction, the apparatus 401 depicted in FIG. 4E operates as a standalone system, independent of any handheld or auxiliary input terminal.

The apparatus 401 includes an outer structural housing formed to be worn on the user's head. It may encapsulate a control unit with a digital signal processor or an embedded AI engine capable of parsing spoken commands issued by the user 402. These commands are received through the microphone 401A, which may be located in a front-facing position near the user's mouth to optimize acoustic clarity and command recognition accuracy.

Microphone 401A may be designed to continuously or periodically listen for voice input in passive or active modes. In passive mode, the microphone may only activate when a wake phrase is detected (e.g., “Start therapy”), whereas in active mode, the apparatus 401 may continuously interpret spoken commands in real time to modify session settings such as duration, intensity, emitter zones, or audio playback parameters.

The apparatus 401 may also include a voice processing module with embedded natural language understanding capabilities that analyze the semantic and emotional context of spoken words. For example, when the user 402 says, “I am feeling tired. I need a quick boost in my awareness,” the system may parse the intent and classify the session request as one corresponding to stimulation of frontal brain regions associated with attention, arousal, and cognitive clarity.

The system may map vocal inputs to predefined therapy profiles or invoke dynamically generated stimulation protocols based on real-time interpretation. The corresponding emitter portals and portions are then activated with specific frequencies, pulse durations, and waveforms stored in internal memory.

In addition to interpreting speech content, the apparatus 401 may also evaluate voice tone, inflection, rhythm, and pitch variability. This acoustic profiling may aid in detecting user emotional states such as fatigue, stress, excitement, or anxiety. The AI system may then factor these inferred states into therapy customization logic.

The apparatus 401 may use its internal logic to select a therapy profile that includes light-based or current-based stimulation of specific cortical areas such as the dorsolateral prefrontal cortex for alertness, or the temporoparietal junction for auditory-motor integration, depending on user input.

Integrated within the apparatus 401 may be an audio output mechanism such as an earphone or speaker 401B. This component may be configured to deliver guided therapy instructions, audio feedback confirming receipt of voice commands, or customized audio therapy such as ambient soundscapes, meditative tones, or binaural beats that complement neurostimulation activities.

In some embodiments, the apparatus 401 may respond to the user's command, “Play relaxation mode,” by initiating a low-frequency emitter pattern in occipital and parietal zones while simultaneously playing ocean wave sounds or slow rhythmic music via the earphone 401B.

Another embodiment may include real-time dialogue functionality where the apparatus 401 acts as a conversational assistant, capable of prompting the user 402 with clarification questions such as, “Do you want to activate Focus Mode or Sleep Mode?” This interactive loop may increase user engagement and aid in precise therapy delivery.

The microphone 401A may incorporate noise-canceling or beamforming technology to eliminate background interference, allowing use in various environments such as offices, homes, or outdoors. Voice recognition algorithms may also be personalized to a specific user's voice profile for secure and accurate interpretation.

The apparatus 401 may store frequently used commands and user preferences locally, allowing it to progressively adapt and personalize stimulation patterns over time without the need for manual re-entry of instructions.

In some configurations, the voice-activated system may be linked to a scheduling engine that tracks previous usage patterns and automatically suggests voice prompts at specific times (e.g., “Would you like to activate your morning focus session?”). This adds a proactive and anticipatory dimension to therapy delivery.

The memory modules integrated within the apparatus 401 may contain a voice command dictionary that may be dynamically updated with new phrases or synonyms recognized during usage, expanding the range of acceptable user input without requiring external configuration.

The voice-only operation mode illustrated in FIG. 4E may be especially beneficial for users with visual impairments, motor disabilities, or those engaged in tasks that restrict manual operation. It allows seamless interaction through natural speech, promoting accessibility and convenience.

In one use case, the user 402 may utter the phrase, “Start 15-minute calm session,” and the apparatus 401 will parse the duration and session type to activate a matched therapy protocol. Afterward, the system may audibly confirm, “Calm session activated for 15 minutes.”

Earphone 401B may be configured to alternate between mono and stereo modes based on therapy type or user preference. Additionally, the audio output may synchronize with neurostimulation pulses to reinforce the effects through multi-sensory entrainment.

The apparatus 401 may further include onboard logging of command history, therapy outcomes, and session durations. This data may later be accessed via a paired device or exported via secure ports for external review, feedback, or medical analysis.

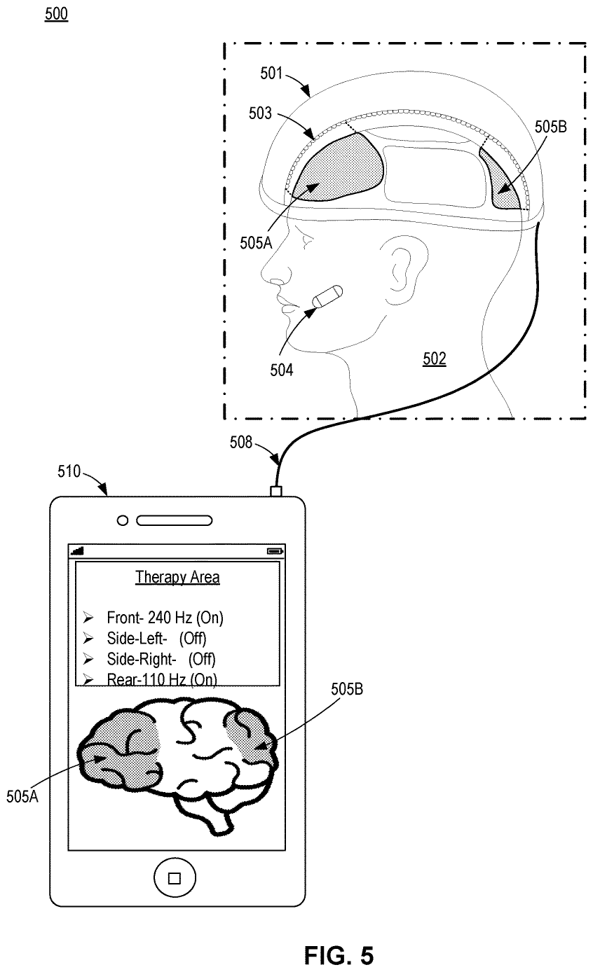

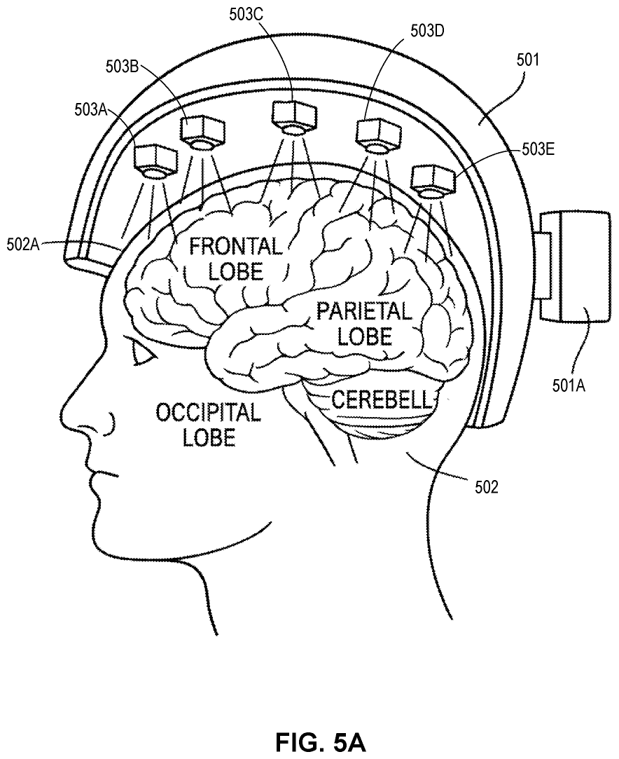

Referring now to FIG. 5, an exemplary system 500 is illustrated comprising a head-mounted neurostimulation apparatus 501 configured to deliver region-specific stimulation to different areas of a user's brain. The apparatus 501 may be worn by a user 502 and interfaces with a user device 510 over a wired connection 508, allowing synchronized display and control of therapy parameters. The illustrated embodiment demonstrates targeting of specific brain regions such as a frontal region 505A and a rear (occipital or cerebellar) region 505B, each being associated with individualized stimulation frequencies as visualized on the display screen of user device 510.

The apparatus 501 may include an integrated energy emitter 503 which includes a plurality of energy emitter portals arranged across the inner surface of the wearable structure. These portals may be configured in combined emitter portions or as discrete zones, depending on therapy design. Each emitter portal may be capable of emitting light or electrical signals to modulate neurological activity within a corresponding brain area. The arrangement allows precision in stimulation planning and delivery.

The user 502 depicted in FIG. 5 may optionally ingest a pre-therapy capsule or pill 504. The pill 504 may include compounds formulated to increase photosensitivity, enhance neuronal energy metabolism, or may cause blood thinning. Example formulations may contain light-absorbing chromophores, such as riboflavin or porphyrin derivatives, that accumulate in neural tissues and optimize the conversion of incident light energy into intracellular activity. Other embodiments may include molecules that enhance mitochondrial responsiveness or membrane ion conductivity.

The ingestion of the pill 504 may result in higher absorption efficiency of the emitted energy by the targeted neural tissues. For example, when a red or near-infrared wavelength is applied through the emitter portals of energy emitter 503, the photosensitizing agent may amplify ATP production within mitochondria, supporting improved neuroplasticity or metabolic recovery in degenerative cases.

Therapy targeting may be determined based on the user's cognitive or therapeutic goal. For example, activation of the frontal brain region 505A may be associated with focus, decision making, working memory, and problem solving. The user device 510 displays an active therapy configuration showing “Front: 240 Hz (On)” suggesting a higher stimulation frequency aimed at enhancing alertness or cognitive load capacity.

Simultaneously, the rear brain region 505B may be targeted for relaxation, visual processing, or motor coordination. The illustrated value “Rear: 110 Hz (On)” denotes a lower frequency, optimized for delta or theta wave entrainment. These rear zones may include the occipital lobe, cerebellum, or posterior parietal cortex, depending on helmet design and emitter placement.

The use of region-specific frequencies allows different neuronal networks to be engaged without mutual interference. In some embodiments, the energy emitter 503 may activate alternate or concurrent pulse patterns across various zones. For example, a first set of emitter portals within the frontal region may pulse continuously, while a second set at the rear may pulse intermittently to establish alternating entrainment rhythms.

The wired connection 508 between the apparatus 501 and the user device 510 facilitates stable data synchronization and shields the user from wireless radiation, offering a non-intrusive and grounded therapy experience. The wire may also serve as a data and power conduit, supplying low-voltage electrical signals for onboard controller and emitter operation.

On the user device 510, visual indicators such as brain maps or animated overlays may illustrate current activity across regions 505A and 505B. For example, upon initiation of therapy, a thermal or dynamic color gradient may appear to show activation intensity or predicted neurovascular engagement in real time.

In some embodiments, the user interface of device 510 may include a customizable dashboard where the user or therapist may assign names, goals, and expected outcomes to each therapy region. For example, region 505A may be labeled “Focus Engine,” while region 505B may be labeled “Relaxation Core.” These settings can be adjusted to user preference or clinical application.

The apparatus 501 may include internal memory or storage that tracks the number of sessions, the regions activated, and the stimulation profiles used. This historical data may be accessed on device 510 to review session efficacy and adapt future stimulation configurations.

Each energy emitter portal within the emitter 503 may be configured to emit light within specific spectral bands, such as blue (400-490 nm), red (620-750 nm), or near-infrared (750-1000 nm), depending on the penetration depth desired and the neurological outcome targeted.

In alternate configurations, the system 500 may modulate not only frequency but also waveform shape—such as sine, square, or triangular waveforms—to produce varied neural excitation patterns across regions 505A and 505B. Multiple emitter regions on apparatus 501 may also be programmed to operate in phase-shifted synchronization. For example, the front and rear emitters may fire in a 180-degree phase shift, promoting cortical oscillatory balance or inter-hemispheric communication.

In some implementations, the user may define routines in which therapy begins with rear-region activation (505B) to induce a relaxed state, followed by frontal-region stimulation (505A) to increase alertness—supporting transition between mindfulness and task engagement states. Each emitter portal within energy emitter 503 may be uniquely addressable, allowing complex sequencing patterns across the cranial surface. The system 500 may exploit this granularity to create sweeping or rotating stimulation patterns across the scalp.

The user device 510 may also display diagnostic overlays representing baseline and stimulated EEG activity, derived from sensors embedded in the apparatus 501. The difference in pre- and post-stimulation activity maps may guide session adaptation. Some therapy routines may designate a third frequency to be applied to lateral brain regions (not shown) to harmonize hemispheric activity. These may be pre-scheduled or initiated based on user-activated combination routines.

Apparatus 501 may include temperature sensors that help monitor scalp or device heat buildup during therapy. This feedback can inform modulation of emitter duty cycle to maintain safe and comfortable stimulation. Energy emitters within 503 may be turned off periodically to simulate pulsed light therapy, where brief darkness intervals are inserted between stimulation periods to prevent desensitization and enhance energy absorption.

The user device 510 may present a timeline slider interface showing when and how each region (e.g., 505A, 505B) is scheduled to activate within a session. The user may adjust timings, add breaks, or insert intermediate therapies dynamically.

In research applications, system 500 may be employed for studying functional connectivity, with regional stimulation patterns mapped to behavioral outcomes logged on user device 510. These patterns may inform clinical practice or cognitive enhancement strategies. In therapeutic use, stimulation of rear region 505B may benefit patients recovering from visual neglect, while frontal stimulation (505A) may support attention regulation in individuals with ADHD or executive function deficits.

In some embodiments, the pill 504 may include time-release formulations such that peak photosensitivity coincides with peak stimulation waveforms, enhancing the synergy between biochemical priming and neuromodulation. The user device 510 may display not only activation frequencies but also show estimated photon absorption or charge transfer levels based on modeled skin, skull, and tissue interactions, allowing more informed adjustments.

In specialized routines, system 500 may alternate between bilateral and unilateral region activation to support lateralization therapy, useful in stroke rehabilitation, hemispheric balancing, or cognitive flexibility training. The apparatus 501 may also support guided user prompts or audio cues (not shown) to prepare the user 502 for regional stimulation. For example, a voice assistant may prompt: “Activating rear cortex at 110 Hz. Please close your eyes and relax.”

Referring now to FIG. 5A, a schematic view of the head-mounted neurostimulation apparatus 501 is depicted, wherein the apparatus may be configured with a plurality of discrete energy emitters 503A through 503E strategically positioned to align with specific brain regions of a user 502. The apparatus 501, worn over the scalp of user 502, is shown in partial section to reveal the internal anatomical layout of the brain and the orientation of energy emitters above the frontal lobe, parietal lobe, occipital lobe, and cerebellum.

The energy emitters 503A-503E are part of a broader emitter system integrated into the inner surface of the apparatus 501, housed structurally within the helmet-like casing. The housing 501A encloses control electronics, including a controller operable with an onboard AI engine configured to govern the selective activation of one or more emitters 503A-503E based on therapy protocols, user interaction, or physiological sensor input.

Each of the energy emitters 503A through 503E corresponds with a distinct neural target. For example, emitter 503A may be aligned with the frontal lobe, which is implicated in decision-making, attention, motor planning, and personality. Emitter 503B targets the anterior parietal lobe, associated with sensory integration and spatial reasoning. Emitter 503C centers over the superior parietal lobe, supporting body coordination and attention shifting. Emitter 503D may be positioned over the cerebellum, which governs fine motor control, balance, and coordination. Finally, emitter 503E may be directed toward the occipital lobe, responsible for visual processing.

The energy emitters 503A-503E may each comprise one or more emitter portals, as described in prior figures, that emit photonic energy (e.g., red, infrared, or ultraviolet light), electromagnetic waves, or low-level electrical impulses in specific, controllable waveforms. The energy profile for each emitter may be defined by parameters including frequency, pulse width, modulation pattern, and intensity level.

In some embodiments, two or more emitters 503A-503E may be activated simultaneously to stimulate distributed cortical networks that are functionally linked. For example, emitters 503A and 503B may be co-activated in a therapy mode aimed at enhancing cognitive focus and sensorimotor integration—relevant in tasks requiring executive control and real-time body coordination.

Simultaneous multi-lobe stimulation may be advantageous in neurological rehabilitation. For example, activation of 503A (frontal lobe) and 503D (cerebellum) may support motor relearning in stroke recovery protocols. The AI engine housed in section 501A may dynamically adjust pulse timing between emitters to create coordinated neuromodulation.

The AI controller within housing 501A may determine optimal emitter configuration and stimulation protocol based on real-time input from integrated physiological sensors (not shown), which may include EEG sensors, HRV monitors, thermal sensors, or user feedback entries. Sensor data may guide emitter activation patterns in closed-loop feedback mode.

Emitter 503C may be used to target parietal lobe functionality for spatial reasoning enhancement or memory consolidation. In memory reinforcement therapy, emitters 503A, 503C, and 503E may be triggered in sequence or as part of a synchronized stimulation profile aimed at enhancing multisensory encoding and long-term memory storage.

The AI engine may select stimulation frequencies based on user cognitive state or desired outcomes. For example, for relaxation therapy, emitter 503D may be activated at a delta frequency (0.5-4 Hz) while emitter 503E pulses at low-frequency theta range (4-7 Hz). For attentional alertness, emitter 503A may be driven at higher beta-range frequencies (13-30 Hz).

Emitters 503A-503E may also operate on independent frequencies for differentiated effect. In some embodiments, 503A may pulse at 20 Hz while 503E pulses at 10 Hz, supporting simultaneous enhancement of executive function and visual system arousal without signal overlap.

The geometry of each emitter 503A-503E may support focused or dispersed stimulation, allowing for micro-targeting of gyri or broader activation across lobe regions. The AI engine may select beam divergence dynamically based on identified therapy goals.

Temporal modulation of emitters may also be employed. In a rhythm therapy protocol, emitters may activate sequentially in a circular pattern (e.g., 503A→503B→503C→503D→503E) to induce cortical phase synchronization or traveling wave phenomena.

The apparatus 501 may also allow user-defined configuration where a user selects therapy focus, and the AI controller maps corresponding emitter sequences. The user may verbally state, “enhance balance and vision,” prompting emitters 503D and 503E to activate with coordinated frequency parameters.

Therapy session logs may capture which emitters were used, for how long, and at what intensity. This data may be used by the AI engine to identify patterns of effectiveness and adapt future therapy sessions accordingly.

The emitters may incorporate wavelength-tunable diode arrays or multi-wavelength LEDs to allow spectral optimization per brain region. For example, 503E may use near-infrared (850 nm) for visual cortex penetration, while 503A uses red light (660 nm) for prefrontal activation.

Each emitter may include built-in sensors for reflection spectroscopy or impedance measurement, allowing feedback about energy absorption or tissue conductivity. Such data may be used to refine emitter tuning in real time. In advanced embodiments, emitter arrays 503A-503E may operate under AI-generated pseudorandom modulation schemes to mimic biological signal variability, supporting plasticity or enhancing stimulus novelty for learning reinforcement.

Therapeutic protocols may include burst-mode delivery where emitters alternate high-frequency pulse trains with recovery intervals. For example, 503B and 503C may alternate rapid beta bursts with delta-range rest periods to encourage neuroadaptive contrast.

Emitters may also be controlled based on user environment, such as ambient light, sound, or motion. For example, when in a dim room, emitter 503E's intensity may auto-adjust to minimize overstimulation while still promoting visual cortex engagement.

In dual-task stimulation, such as walking and problem-solving, emitters 503D and 503A may be engaged in split-pattern configuration, maintaining cerebellar motor calibration alongside prefrontal executive support.

Social cognition therapies may target emitters 503A and 503C together, as these regions are implicated in empathy, emotional interpretation, and perspective-taking. Frequencies and timing may be synchronized to user verbal or visual stimuli. In some cases, the emitters may be pulsed in accordance with physiological rhythm inputs, such as heartbeat or respiration. For example, 503D stimulation may track breath rhythm to induce parasympathetic activation and reduce anxiety.

Customization of emitter sets may allow therapy personalization by demographic parameters such as age, gender, or neurotype. AI-driven emitter scheduling may be tailored to ADHD profiles, ASD regulation, or age-related cognitive decline. Emitters 503A-503E may be assigned contextual names (e.g., Vision Stimulator, Balance Anchor) within the user interface, improving comprehension and user engagement during therapy planning.

The apparatus 501 may also support offline pre-programming of emitter routines stored in housing 501A's memory, allowing autonomous function in field environments without active user input or continuous device control. In collaborative therapy, user 502's emitter profile may be mirrored across multiple systems to coordinate group neurostimulation sessions, useful in synchronized mindfulness, cognitive training, or multiplayer virtual environments.

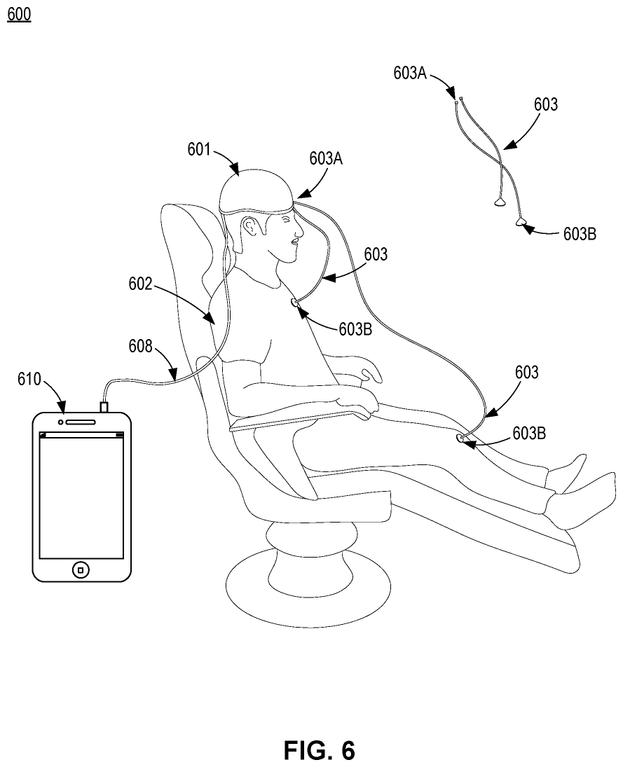

Referring now to FIG. 6, an exemplary system 600 is illustrated wherein a head-mounted neurostimulation apparatus 601 is utilized by a user 602 in a seated configuration that reflects an accessible, non-clinical environment, such as a domestic or home setting. The apparatus 601 may be adapted to be operatively connected with a plurality of peripheral sensing probes 603 as well as a user device 610 via a wired connection 608. This embodiment highlights the versatile and decentralized application of the therapy apparatus without requiring bulky or immobile laboratory infrastructure.

The neurostimulation apparatus 601 may include a wearable housing configured to accommodate emitter assemblies, internal control circuits, AI logic processors, and multiple connection ports for external peripherals, such as the sensing probes 603. The apparatus 601 may be shaped to conform to a user's scalp and may integrate internal supports or pads for comfort and positional consistency.

The sensing probes 603 each include a connector end 603A and a sensing tip 603B. The connector ends 603A are removably insertable into dedicated connection ports located on the outer surface or peripheral ridge of the apparatus 601. These ports may be magnetic, snap-in, or twist-lock based to facilitate rapid attachment and secure retention during user activity or rest.

The sensing tips 603B are configured to be affixed to various body parts of the user 602, such as the chest, upper arms, thighs, knees, or wrists. These tips may be adhesive-backed, strap-based, or use biometric pads embedded in garments or recliner surfaces. The sensing tips 603B may contain electrodes, optical readers, pressure sensors, or thermographic materials to acquire continuous physiological data.

Each sensing probe 603 may be constructed to relay data from its tip 603B to the apparatus 601 in real time. The types of data collected may include heart rate, heart rate variability, body temperature, galvanic skin response, skin conductivity, muscle tone, or localized blood oxygen levels. Such data may be used as input parameters in closed-loop modulation algorithms executed by the controller embedded within the apparatus 601.

In some configurations, a probe 603 placed over the user's chest detects heart rate and synchronizes stimulation pulses to cardiac cycles. For example, during relaxation protocols, energy pulses from the apparatus 601 may align with the user's diastolic phase to promote parasympathetic entrainment. Likewise, rapid heart rate may cue a downregulation of frequency or shift in the active emitter regions.

A sensor 603B placed near the knee or lower limb may capture movement artifacts or tremor signals. In use cases involving motor disorder therapy, the apparatus 601 may shift activation from frontal emitters to cerebellar ones based on the sensor's feedback, supporting dynamic neuromotor modulation.

The probes 603 may also collect baseline physiological data at session start and deliver comparative readings at various intervals, enabling the AI engine within apparatus 601 to measure therapeutic effectiveness and adjust protocols on the fly.

The apparatus 601 may store sensor data locally or transmit it to the user device 610 over wired connection 608. The wired link reduces electromagnetic noise and latency, allowing near-instantaneous feedback loops. In another embodiment, the apparatus 601 may function wirelessly using encrypted Bluetooth or near-field communication protocols if user consent is obtained.

The user device 610 may feature a graphical interface for visualizing data from the probes 603. For example, live charts of heart rate variability, skin temperature, or muscle activity may be displayed in tandem with a session progress bar. This feedback may improve user adherence and self-awareness.

In some embodiments, probes 603 with sensing tips 603B use dual-function materials capable of both detecting and emitting low-level photonic signals. This enables interactive light-based measurements and therapy from the same probe site, enhancing multisite stimulation strategies.

The data received from the sensors may be used to modulate the selection and combination of emitter portals within the apparatus 601. For example, elevated skin temperature combined with high heart rate may trigger the AI controller to activate parietal emitters at low frequency to promote relaxation.

Sensor-guided intensity regulation may be implemented whereby energy output is increased or reduced based on user's physiological tolerance thresholds derived from historical data stored in memory or recent probe readings.

Probe 603 placement may be user-defined or AI-recommended based on therapy type. For memory enhancement, probes may be suggested at wrist and neck locations for vascular flow monitoring, while motor therapy may utilize lower limb sensors.

Each connector end 603A may contain embedded identification chips that communicate their type and functional role to the apparatus 601 upon insertion. This allows the apparatus 601 to auto-detect the type of sensor connected and appropriately configure data channels and signal processing routines.

In some versions, probes 603 may be color-coded or shape-coded to aid users in self-installation without requiring technical expertise. The apparatus 601 may confirm proper connection and functionality using visual or audio feedback.

Session data collected from probes 603 and stored in device 610 or onboard storage of apparatus 601 may be reviewed post-session via reports. These reports may indicate session adherence, physiological variability, and AI-recommended therapy adjustments. In another embodiment, the probes 603 are detachable and swappable, with a library of specialized tips 603B that include optical oxygen sensors, EMG electrodes, skin impedance sensors, or sweat analyzers. These modules provide additional customization based on user health status.

Therapy modification based on probes 603 may include not only brain area targeting but also pulse train variation, waveform shape changes, emitter rotation order, or activation duty cycles, making therapy highly adaptive and time-responsive. Some probes 603 may include ambient sensors to detect environmental context—such as room light, noise, or temperature—which may influence emitter selection. For example, increased room temperature may reduce energy intensity automatically.

In group therapy settings, data from multiple users' probes may be aggregated by a host device for synchronized therapy sessions with personalized modulation embedded into each apparatus 601.

The apparatus 601 may also feature LED indicators linked to probes 603 that glow or blink to reflect real-time sensor data such as heart rate zone or thermal status, supporting visual biofeedback. Probes 603 may also function as training sensors where user learns to consciously influence physiological markers like breath rate. Apparatus 601 may then reward the user with neurostimulation changes based on performance goals.

The AI logic within the apparatus 601 may tag certain body signals as pre-therapy cues, such as a threshold drop in heart rate, upon which it starts therapy automatically without manual input. In offline use, the apparatus 601 may use default sensor-based routines stored in firmware, activating or suppressing emitters based on real-time probe feedback without involving user device 610.

Probe wires (603) may be retractable, wound, or adjustable in length to accommodate different body sizes and postures, supporting use in seated, lying, or ambulatory positions. When used in conjunction with memory enhancement protocols, sensors 603B may measure galvanic skin response to emotionally salient stimuli and adjust emitter focus to reinforce long-term retention.

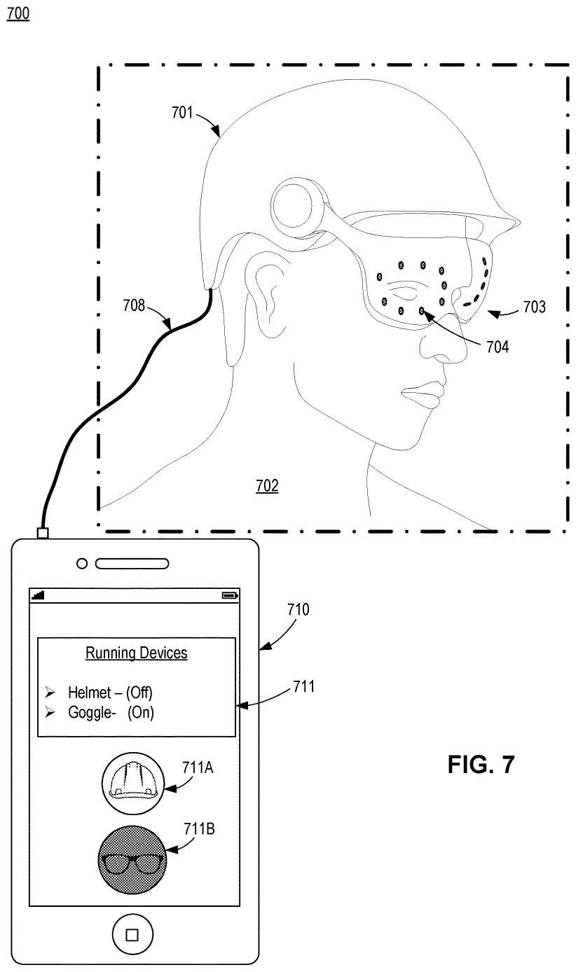

Referring now to FIG. 7, an exemplary system 700 is illustrated, wherein a head-mounted neurostimulation apparatus 701 includes a goggle-type wearable device 703 designed for placement over the facial and ocular region of a user 702. The apparatus 701 is operatively connected to a user device 710 via a wired connection 708, thereby enabling controlled activation of the various subsystems of the apparatus 701, including a helmet-type portion and a facial goggle-type portion. The illustrated configuration permits the system 700 to function as a modular neurostimulation platform suitable for applications involving vision stimulation, emotional state balancing, and localized therapy to facial musculature or cranial nerves.

The goggle 703 includes a plurality of energy emitters 704 integrated across the inner-facing surface of the goggle frame. These energy emitters 704 may be spatially distributed to align with periorbital zones including the supraorbital ridge, zygomatic arch, nasal bridge, and temple regions, while deliberately avoiding direct alignment with the corneal axis of the eyes. This spatial configuration prevents direct optical stimulation, thereby supporting user safety during extended sessions.

The emitters 704 may emit light within specific non-harmful spectra, such as low-intensity near-infrared (800-900 nm) or red light (620-670 nm), selected for their low retinal penetrability and therapeutic bioactivity. Beam angles of the emitters 704 may be configured such that rays are refracted obliquely away from the pupil and toward areas surrounding the eye sockets.

In some embodiments, each emitter 704 may be housed in a recessed dome or covered with a diffuser lens to scatter emission and prevent localized high-intensity exposure. This may allow emission power to remain therapeutic while protecting ocular structures from adverse cumulative exposure.

The goggle 703 may be formed from transparent or translucent materials that permit ambient light ingress and user visibility while serving as an anchoring structure for the emitters 704. Example materials may include flexible silicone-based polymers or optical-grade polycarbonate with embedded micro-LED mounts.

The head-mounted apparatus 701 may house additional subsystems, including sensors, memory storage, and a controller equipped with an AI engine capable of coordinating stimulation strategies across helmet and goggle subsystems. These systems may be housed within peripheral housing modules such as ear-mounted housings or the rear enclosure of the helmet.

The user device 710 may be used to configure the activation states of multiple device components. As illustrated, a user interface 711 is presented on the user device 710 and may display real-time operational status of each subsystem, including a helmet status 711A and a goggle status 711B. A user may selectively activate or deactivate either module through the interface 711 depending on therapy goals or comfort preferences (by simply selecting helmet 711A and goggle 711B).

In some embodiments, the goggle 703 may be used as a stand-alone neurostimulation module, particularly for treatments targeting visual fatigue, photophobia, facial neuralgia, or ocular stress recovery. Alternatively, the goggle 703 may operate in conjunction with the helmet 701 in synchronized or alternating patterns.

The wired connection 708 between the apparatus 701 and the user device 710 allows for low-latency command transmission and avoids the potential biological interference of wireless electromagnetic signals in close proximity to cranial structures.

The goggle 703 may include additional embedded sensors to monitor parameters such as skin temperature, blink rate, pupillary diameter, or facial muscle tension. Sensor data may be fed into the controller within the apparatus 701 for real-time therapy adaptation. In one use case, a user 702 undergoing therapy for stress-induced migraine may activate only the goggle 703 using the user interface 711, and the AI engine may initiate a pulse sequence through emitters 704 targeted at the frontal and periorbital regions to modulate trigeminal nerve activity.

In another embodiment, vision enhancement protocols may involve pulsating red-light stimulation delivered through the emitters 704 during low-light adaptation phases, where retinal sensitivity enhancement is desirable but must be achieved indirectly.

Emitters 704 may operate on distinct frequencies or intensities to facilitate alternating bilateral stimulation. For example, left-side emitters may operate on 10 Hz while right-side emitters activate at 12 Hz, creating cross-hemispheric entrainment effects in visual cortices. To address digital eye strain, the goggle 703 may use pulse modulation in emitter 704 to simulate natural light fluctuations, thereby triggering intrinsic rest mechanisms in the ciliary muscles and aiding in visual focus recovery.

The goggle 703 may feature adjustable arms or nose-bridge mounts allowing emitter alignment to be adapted to different face geometries. This provides optimal placement of energy emitters 704 over targeted muscle groups and cranial nerve junctions. The controller within apparatus 701 may also support pattern-based therapy, wherein emitters 704 are activated in wavefront or clockwise/counterclockwise sequences, corresponding to user-set protocols for stress reduction, cognitive clarity, or ocular detox routines.

A user 702 may verbally or via touch input request activation of only the goggle 703 module if facial therapy is desired during travel or resting situations. The apparatus 701 may enter a power-saving mode by disabling the helmet and focusing solely on localized front-facing emitters.

The energy emitters 704 may be coupled with haptic feedback modules embedded within the goggle 703 to provide tactile cues that reinforce therapy stages or alert the user to transitions between phases. Emitters 704 may also be paired with audio cues generated through ear-mounted speakers or bone-conduction headphones to create a multimodal neurostimulation environment involving synchronized visual and auditory entrainment.

The user interface 711 may be configurable to show real-time emitter maps, waveform plots, session duration, and sensor feedback related to the active goggle 703 operation. Each of the operational elements of the goggle and helmet may be independently tunable. In another configuration, goggle 703 may serve as a sleep enhancement function. Emitters 704 may generate slow ramp-down sequences mimicking dusk lighting to stimulate melatonin release via indirect activation of circadian pathways.

The goggle 703 may be compatible with other wearable technologies. For example, synchronization with smartwatch-based data (e.g., HRV or sleep stage) may allow the controller in apparatus 701 to initiate stimulation only during ideal circadian windows. The apparatus 701 may store user profiles including historical usage of the goggle 703, sensitivity tolerances, preferred emitter sequences, and feedback loops for rapid configuration reuse in subsequent sessions.

The goggle 703 may also feature a retractable or removable design that allows it to be detached when not in use or replaced with alternate modules such as infrared-only emitters or light therapy panels for seasonal affective disorder treatment. The energy emitters 704 may be arranged in concentric or radial patterns around the periorbital zones to promote uniform field dispersion, with customizable geometric layouts that the AI engine may tailor per user needs.

For use cases in visual rehabilitation, the goggle 703 may support lateralized stimulation where emitters 704 on only one side of the face are activated to encourage neuroplasticity and compensatory reorganization in cases of visual field loss. The AI controller may also detect user emotional states through biometric sensors and respond by engaging the goggle 703 with facial therapy stimulation to relieve muscle tension and promote relaxation. To accommodate low-light environments, the goggle 703 may include auto-dimming functionality and ambient light sensors, which help the AI adjust the emitter 704 output to levels appropriate for environmental lighting conditions.

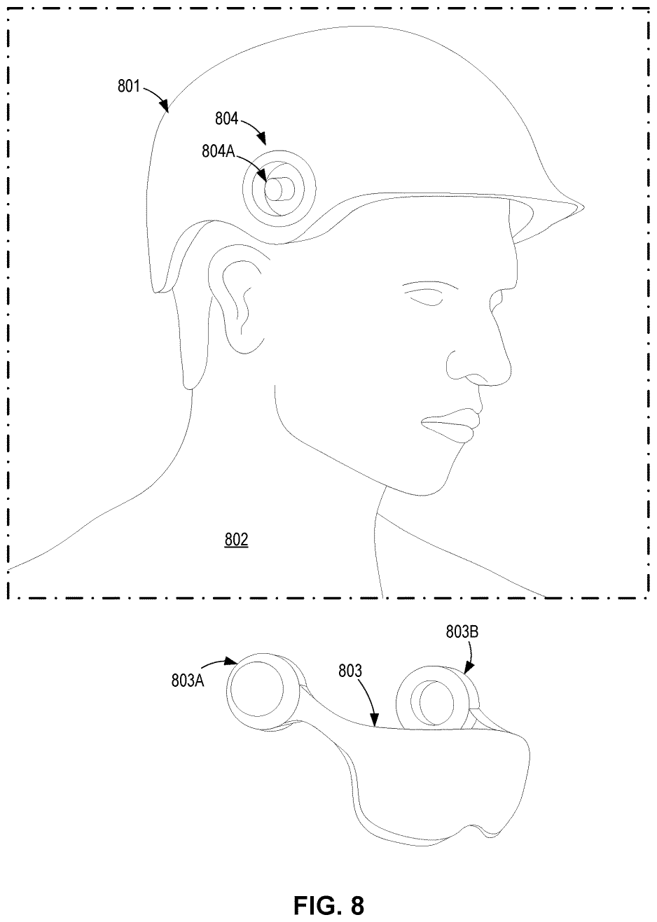

Referring now to FIG. 8, an exemplary neurostimulation apparatus 801 is illustrated, comprising a helmet portion configured for secure placement over the head of a user 802, and a removably attachable goggle portion 803, enabling modular expansion of functionality. The apparatus 801 may be designed to support multiple therapy modules and to accommodate interchangeable components for enhanced personalization and versatility in neurostimulation therapy.

The helmet portion of the apparatus 801 may include a goggle attachment interface 804, which includes one or more mechanical and electrical coupling elements designated as interface connectors 804A. These connectors 804A are strategically positioned on the lateral side of the helmet to allow ergonomic placement of the goggle portion 803 while maintaining reliable structural support and data transmission integrity.

The goggle portion 803, which may be configured for placement across the facial region of the user 802, may include mating connectors 803A and 803B that engage with the interface connectors 804A of the helmet 801. The connectors 803A and 803B may be embodied as snap-lock, magnetic latch, twist-lock, or slide-in bayonet style interfaces to facilitate fast and secure attachment and detachment by the user.

The mechanical coupling provided by connectors 803A and 803B may include alignment pins, interlocking teeth, or contour-fitted geometry to guarantee stable physical attachment during therapy sessions, even during user movement. This allows the goggle 803 to be firmly suspended in front of the user's eyes without requiring an independent strap or harness.

The electrical component of the interface connectors 804A facilitates bidirectional signal flow between the goggle 803 and the helmet 801. In some embodiments, power lines from a battery or energy regulation circuit housed within the helmet are routed to the goggle 803 via interface connectors 804A, thus supplying continuous operational current to any energy emitters embedded in the goggle structure.

The goggle 803 may contain a plurality of energy emitters, as described in relation to earlier figures, strategically placed around ocular regions for therapy involving vision stimulation, eye strain reduction, or stress management. Activation of these emitters may be controlled via the helmet's internal controller through communication links integrated into the connectors 804A.

In some embodiments, the goggle 803 also includes one or more sensors positioned for data collection from the user's facial region. These sensors may include thermal sensors, facial muscle tension detectors, blink rate monitors, or photoplethysmographic sensors for detecting blood volume changes. Sensor data may be transmitted via the same interface connectors 804A to the helmet's onboard processing unit.

Power and data lines integrated within connectors 803A, 803B, and 804A may be arranged as spring-loaded contact pads, pogo pin arrays, or magnetically aligned conductive surfaces to facilitate consistent connection quality even under minor shifts during use.

The apparatus 801 may recognize the attachment of the goggle 803 via detection circuits embedded within interface connectors 804A. Upon successful detection, the onboard controller may initiate a configuration routine to activate the goggle 803, apply default emitter parameters, and begin data polling from integrated sensors.

The helmet 801 may optionally allow the goggle 803 to operate semi-independently by granting access to a subset of its memory and processing resources. This allows autonomous emitter management when low-power routines are required or when operating under preprogrammed schedules.

In some embodiments, removal of the goggle 803 results in a software interruption signal sent to the controller, prompting safe shutdown of power output to the goggle interface, memory buffer preservation, and thermal cooldown routines to preserve component longevity.

The removable nature of the goggle 803 enables users to swap between different goggle modules. For example, a first goggle may be optimized for general wellness therapy while a second may be designed with emitters focused on the temples and cheeks for migraine mitigation. The helmet 801 may identify goggle variants via encoded chip embedded in the connectors.

Users 802 may also manually select goggle activation status from a paired user device (not shown in this figure), where the interface displays status indicators for helmet and goggle portions, along with live readouts of emitter frequency, intensity, and sensor readings from the goggle 803.

In another embodiment, the goggle 803 may feature transparent or semi-transparent lens areas, allowing the user 802 to retain visual capability during therapy. The energy emitters, housed within or around the lens periphery, may be angled or diffused to prevent direct corneal exposure.

The modular nature of the interface connectors 804A may also allow firmware upgrades, wherein the helmet controller transfers updated control protocols or waveform libraries to memory located within the goggle 803. Such configurations support evolving therapy routines and adaptability to new use cases.

In safety-focused configurations, a fail-safe mechanism may be incorporated into connectors 803A-803B such that energy delivery is automatically disabled if the physical connection is unstable, dislodged, or exposed to moisture.

To accommodate a variety of head shapes and face contours, the goggle 803 may be articulated through one or more pivot joints located adjacent to the connectors 803A-803B. This allows the user to angle the goggle downward or outward for comfort without compromising the electrical connection.

The goggle 803 may also incorporate haptic vibration elements controlled via the same electrical link to enhance multisensory therapy experience in applications related to mood enhancement, eye strain therapy, or synchronized breathing guidance.

The illustrated apparatus 801 and goggle 803 thus represent a flexible and modular system capable of extending core neurostimulation functionalities to include facial and vision-related therapy without redundant hardware. FIG. 8 demonstrates the integration of mechanical stability and electrical interoperability between subsystems using compact, scalable interface connectors.

This arrangement allows therapy regimens to evolve with the user's needs by upgrading or replacing just the goggle portion 803 while maintaining backward compatibility with the helmet portion 801. Such configurations promote long-term device utility and cost-effective personalization in neurostimulation applications.

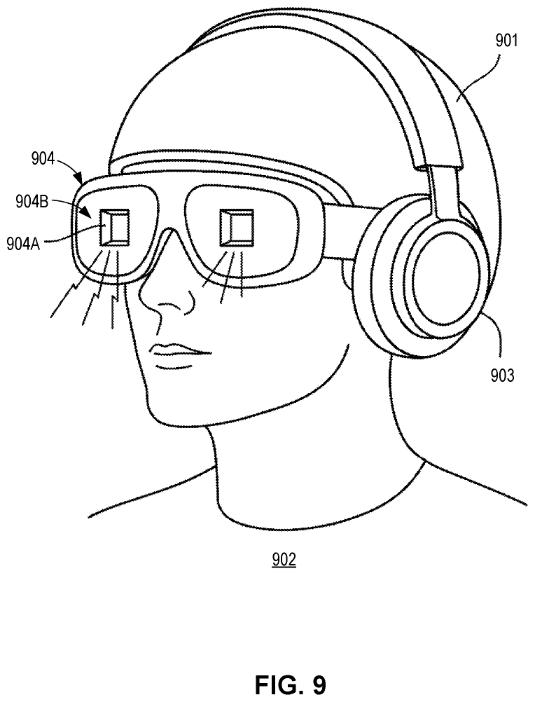

Referring now to FIG. 9, an exemplary neurostimulation system is illustrated comprising a wearable configuration that may include a helmet 901, a goggle-type neurostimulation device 904, and an integrated speaker assembly 903, collectively configured for delivering multimodal therapy to a user 902. The goggle 904 features a see-through lens portion 904A and a surrounding energy emitter portion 904B configured to emit targeted light stimulation to ocular-adjacent areas without obstructing vision.

The see-through lens portion 904A is fabricated using optically clear polymers such as polycarbonate or TR90, optionally coated with anti-glare and blue-light filtering layers. This transparent lens structure allows users 902 to retain full situational awareness and visual capability while undergoing stimulation therapy, making the device suitable for use in daily activities, relaxation, or guided meditation environments.

The energy emitter portion 904B surrounds the perimeter of the lens portion 904A and may include an array of embedded micro-LEDs, OLED strips, or miniaturized near-infrared (NIR) emitters. The emitters are configured to deliver regulated light energy to the upper cheeks, temples, brow area, and bridge of the nose, corresponding with known trigger points and neurological pathways involved in mood, stress, and visual processing.

The emitter portion 904B may emit light in specific therapeutic wavelengths, such as 630-670 nm for red light or 800-900 nm for NIR. These wavelengths have been associated with photobiomodulation effects including mitochondrial ATP production, nitric oxide release, and increased local blood circulation, contributing to relaxation and ocular recovery.

In some embodiments, the goggle 904 may include angled emitter housings within the emitter portion 904B to direct beams away from the user's direct line of sight. This helps prevent retinal overstimulation and allows longer durations of safe therapy. Reflective baffles or diffusive membranes may be incorporated to scatter the light evenly across facial surfaces.

The speaker assembly 903 may be coupled laterally with the helmet 901 and positioned over the user's ears. The speaker 903 may be configured to emit calming sounds, binaural beats, verbal guidance, or neuro-acoustic stimulation patterns that are temporally synchronized with light pulses emitted by the goggle 904. This enables a cohesive multimodal therapy session targeting auditory and visual pathways simultaneously.

In some implementations, the helmet 901 may comprise a housing for power supply, controller circuitry, and wireless communication modules. The controller may drive both the speaker 903 and the emitter array 904B, based on preprogrammed protocols or real-time sensor feedback.

The emitter portion 904B may operate in multiple patterns such as alternating blink, sinusoidal pulse, or frequency sweep. Each pattern may correspond to a specific cognitive or emotional state enhancement program—e.g., increasing alertness, reducing fatigue, or stabilizing mood.

Sensor arrays may be optionally embedded in the frame of the goggle 904 to detect blink rate, pupil dilation, or eye movement. These parameters may be processed by an AI engine to dynamically adjust light output in real time. For example, increasing blink rate may trigger a dimming routine to allow visual recovery.

In certain embodiments, the goggle 904 may support detachable lens modules, allowing users to switch between different tint levels or lens prescriptions. This enables users 902 to wear the apparatus in a variety of ambient lighting conditions without visual discomfort.

The helmet 901 may contain magnetic latch points or snap-fit connectors to mechanically secure the goggle 904. Electrical power and data transmission pathways between the helmet and goggle may be realized using pogo-pin terminals, conductive hinge joints, or flexible printed circuit ribbons.

The user 902 may activate and manage therapy sessions through a companion mobile application (not shown), which may present visualization of therapy parameters such as current waveform, stimulation duration, and facial region targeting. The app may also allow adjustment of sound levels emitted by speaker 903.

In some embodiments, the helmet 901 and goggle 904 may support onboard memory storage for recording session data and user preferences. Such storage may enable resume-on-wear functionality where the last-used therapy mode is automatically reinitiated upon donning the apparatus.

The goggle 904 may also feature anti-fogging technology such as embedded heating films or vent channels to preserve clarity of vision in varying environmental conditions. In cold environments, mild heating may additionally aid circulation and comfort during therapy.

The speaker 903 may be realized as a bone conduction driver embedded in the helmet 901. This would allow users to experience auditory input without blocking environmental sounds, maintaining safety and awareness during ambulatory use.

In applications involving ocular fatigue from prolonged screen exposure, the goggle 904 may deliver periodic red-light pulses via emitter portion 904B to stimulate the lacrimal gland region and enhance tear production. This provides relief in users with dry eyes or screen-induced eye strain.

In alternate configurations, the emitter portion 904B may also support UV-free blue light stimulation (e.g., 470 nm) for cognitive alertness boosting in low-energy environments. Such usage may be scheduled during morning sessions to support circadian rhythm alignment.

For neuro-cognitive enhancement, the goggle 904 and helmet 901 may coordinate cross-modal entrainment, wherein the light pulses and auditory beats are harmonized at frequency ranges associated with alpha or theta wave neural activity.

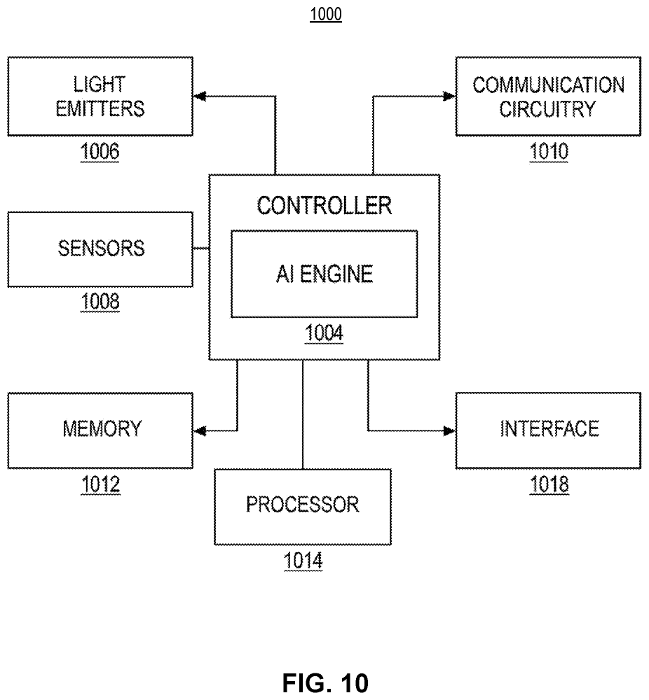

Referring now to FIG. 10, a block diagram of an exemplary neurostimulation apparatus 1000 is illustrated. The apparatus 1000 includes a system architecture configured to execute, control, and adapt various stimulation therapies delivered through wearable or modular neurostimulation devices. The apparatus 1000 may include a controller 1004 integrated with an AI engine, which serves as the central logic and decision-making unit interfacing with a variety of peripheral and core subsystems including light emitters 1006, sensors 1008, communication circuitry 1010, memory 1012, processor 1014, and user interface 1018.

The controller 1004 may be operatively responsible for coordination and supervision of the therapy protocols applied through the neurostimulation apparatus 1000. The controller 1004 may be embodied in a microcontroller, system-on-chip (SoC), or custom-built embedded controller. The controller 1004 may include internal logic for synchronizing emitter activation, receiving sensor data, managing user configurations, and executing therapy schedules stored within the apparatus.

In some embodiments, the controller 1004 includes a dedicated AI engine embedded therein. The AI engine may include machine learning models, neural network inference frameworks, or probabilistic decision matrices used to determine therapy strategies. These strategies may be adapted based on incoming sensor data, user behavior, user preferences, environmental cues, and historical session analytics.

The light emitters 1006 represent one or more energy emission modules, which may include but are not limited to, infrared diodes, red-light LEDs, laser-based photonic sources, or OLED-based surface emitters. These emitters may be mounted within the inner shell of a helmet, on a goggle frame, or embedded in peripheral attachments targeting body regions beyond the head. The controller 1004 regulates the duty cycle, intensity, frequency, and emission patterns of these light emitters 1006 according to therapy requirements.

The sensors 1008 operatively coupled to the controller 1004 may include a plurality of biofeedback mechanisms such as EEG electrodes, heart rate sensors, skin temperature sensors, galvanic skin response detectors, photoplethysmography units, and motion sensors. These sensors may be embedded in different parts of the apparatus such as within the helmet lining, goggle rim, or contact points along the chest, neck, and limbs.

The controller 1004 continually monitors real-time physiological data captured by sensors 1008 to assess user response to therapy, detect onset of fatigue, stress, or emotional variation, and adjust stimulation parameters dynamically. In some embodiments, the sensor data may be utilized by the AI engine to derive context-aware therapy adjustments without human intervention.

Communication circuitry 1010 provides bidirectional wired and wireless connectivity between the apparatus 1000 and external systems such as user devices, remote servers, cloud platforms, or remote health care professionals. The communication circuitry 1010 may include USB, Bluetooth, Wi-Fi, Zigbee, or proprietary low-energy radio modules. The AI engine may selectively engage communication interfaces to transmit analytics, receive configuration updates, or respond to remote commands.

In certain configurations, the communication circuitry 1010 may remain deactivated during active stimulation therapy to avoid exposure to electromagnetic radiation, and only enabled post-session for data transmission or device updates. The user may control this behavior via interface 1018 or companion app on a user device.

The memory 1012 operatively connected to the controller 1004 stores device firmware, stimulation routines, session logs, user profiles, environmental adaptation models, and training datasets used by the AI engine. The memory 1012 may comprise volatile (e.g., DRAM) and non-volatile (e.g., flash, EEPROM) components. Some embodiments support cloud-synced memory for therapy backup and cross-device migration.

The processor 1014 may be coupled to the controller 1004 and may be configured to perform additional computation tasks such as signal preprocessing, Fourier transformations of EEG input, derivation of HRV indices, or thermal mapping of sensor input. In some embodiments, the processor 1014 performs load balancing with the AI engine, offloading intensive computation to maintain real-time responsiveness.

The user interface 1018 may include all input/output elements allowing user interaction with the neurostimulation apparatus 1000. The interface 1018 may incorporate tactile buttons, capacitive touch panels, rotary dials, or vocal command inputs through microphones. In addition, visual feedback may be provided via LED indicators, OLED displays, or graphical user interfaces displayed on connected external devices.

The user interface 1018 may include physical connection ports (e.g., 104) for attaching peripheral components such as user devices (e.g., 210), external sensing probes (613), interchangeable goggle modules (703), energy patches for non-head regions, power source for charging a battery of the apparatus (1000), or audio accessories. Ports may be standardized (e.g., USB-C) or custom for secure locking and waterproofing.

The AI engine embedded within controller 1004 may apply supervised or unsupervised learning algorithms to detect user patterns such as therapy adherence, responsiveness, or fatigue. Based on these insights, the AI engine may automatically select appropriate emitter zones, modulation frequencies, and timing sequences for subsequent therapy sessions.

The AI engine may further process sensor signals to detect physiological states such as drowsiness, anxiety, or alertness based on statistical models or trained classifiers. Upon detection of specific states, it may initiate energy emission from corresponding emitter arrays tuned to therapeutic frequencies known to support regulation of those states.

In a particular embodiment, the AI engine can generate predictive models that estimate the outcome of different stimulation settings on future session performance or user well-being metrics. The apparatus 1000 may thus personalize therapy delivery in real-time using a closed-loop feedback system.

For example, if the AI engine detects consistently high skin temperature readings from sensors 1008 after stimulation to the frontal cortex, it may reduce emitter power in that zone and reroute stimulation to adjacent areas using different emitter arrays under light emitters 1006.

The apparatus 1000 may support multiple operation modes, such as manual therapy mode (where user manually selects parameters), adaptive mode (where AI engine selects based on real-time input), and training mode (where the AI engine learns optimal settings based on trial and error with feedback). These modes may be selected using the user interface 1018 or remotely.

Power management protocols may also be governed by the controller 1004, using data from sensors 1008 and processor 1014 to determine optimal shutdown, standby, or charging behavior. Thermal sensors may prevent overheating by regulating emitter usage patterns or reducing intensity.

User authentication features may be incorporated into the user interface 1018 using biometric sensors embedded in the apparatus 1000, such as fingerprint, voiceprint, or facial recognition modules, allowing secure access to therapy configurations. In some embodiments, the communication circuitry 1010 may receive real-time health alerts from external applications, such as reminders for hydration or posture corrections, which are then used by the controller 1004 to modulate therapy timing or content. Remote access via communication circuitry 1010 allows medical professionals to monitor therapy progress, adjust protocols, and receive compliance reports without requiring the user to visit a clinic. This also allows caregivers to track user wellness.

In some embodiments, controller 1004 may be linked to environmental sensors that measure light levels, sound, temperature, or air quality. This data may be incorporated by the AI engine to provide context-aware stimulation, such as dimming stimulation levels in dark environments or enhancing alertness in noisy settings. The controller 1004 may support firmware upgrades via communication circuitry 1010. This enables deployment of new therapy models, updated security patches, and enhanced AI capabilities as they are developed.

The neurostimulation apparatus 1000 may also be configured with redundancy logic, where failure of any subsystem (e.g., communication circuitry 1010 or sensors 1008) does not halt the therapy but triggers fallback routines executed by processor 1014. The AI engine may also factor in subjective user feedback, collected through interface 1018 or external apps, to refine its internal models. Feedback scores can influence emitter selection, therapy length, or rhythm of light modulation.

In one scenario, the AI engine identifies a therapy pattern that reduces anxiety markers in the sensor data within five minutes of use and labels it as a preferred mode. This mode may be proposed automatically in future sessions or set as a default. The architecture represented by FIG. 10 allows multiple forms of energy emission to be managed simultaneously, including combinations of red, blue, and infrared light, with each emitter array under light emitters 1006 tuned independently based on AI computation. The sensors 1008 may include impedance sensors to detect whether the apparatus 1000 is worn correctly, thereby initiating stimulation only when proper placement is confirmed, avoiding unintended exposure or misuse.

The system architecture is scalable and may be extended with additional sensor types, emitter modules, or external computation units using the modular interface provided via communication circuitry 1010 and user interface 1018. User interface 1018 may also be used to activate emergency functions, such as calling a caregiver or triggering an automatic shutoff, using predefined gestures or button sequences. In certain embodiments, the AI engine supports multilingual interaction, enabling users to receive voice prompts and configure settings in their preferred language using speaker and microphone subsystems integrated into the apparatus.

The controller 1004 may also analyze long-term session data and detect deviations from established therapy norms, prompting alerts or recommendations for re-evaluation or adjustment. In hybrid use cases, such as guided meditation with simultaneous light stimulation, the AI engine may synchronize light emitters 1006 with rhythmically modulated audio playback using speaker systems driven via processor 1014.

The configuration shown in FIG. 10 enables closed-loop therapy management, where user input, real-time sensor readings, memory-stored profiles, and AI-guided decision logic interact in a seamless feedback cycle governed by controller 1004. Such integration of distributed sensor-actuator coordination with adaptive intelligence allows the neurostimulation apparatus 1000 to be applied across a wide spectrum of use cases including therapeutic, recreational, cognitive training, stress reduction, and sensory rehabilitation scenarios.

In some embodiments of the present invention, a neurostimulation apparatus (1000) may comprise an artificial intelligence (AI) engine configured to manage, analyze, and adapt therapy sessions delivered via one or more energy emitters disposed within a wearable device. The AI engine may be implemented in multiple locations, including but not limited to: (i) locally within the wearable apparatus (e.g., helmet or goggle-based configuration); (ii) within a connected user device (e.g., smartphone, tablet, or computing terminal); and/or (iii) on a remote server communicatively linked to the apparatus or user device through wireless protocols such as Wi-Fi, Bluetooth, LTE, or satellite communication.

In an embodiment where the AI engine is integrated into the wearable neurostimulation apparatus itself, the controller of the apparatus may include embedded processing hardware and firmware capable of executing machine learning models. Such models may be stored within onboard memory and optimized for inference at the edge, i.e., without requiring constant data transmission to external computing systems. This configuration supports standalone operation, particularly advantageous for users preferring offline therapy sessions or seeking to minimize wireless exposure during stimulation.

In another embodiment, the AI engine may reside within a connected user device such as a smartphone or tablet. In this scenario, the wearable apparatus transmits real-time sensor and therapy session data via a wired or wireless interface to the user device, which then performs data preprocessing, classification, and decision-making using a local AI module. The user device may run dedicated software configured to execute neural network models, decision trees, probabilistic models, or reinforcement learning routines that determine appropriate stimulation parameters based on physiological states and therapy objectives.

In yet another embodiment, the AI engine may be cloud-based and hosted on a remote server. In this configuration, therapy data collected by the apparatus and/or user device may be periodically or continuously transmitted to the remote server, where a centralized AI system performs higher-order analysis, longitudinal pattern tracking, multi-user comparison, and continuous model refinement. This remote AI engine may be configured to generate optimized therapy protocols, push firmware updates, or transmit alerts to caregivers or medical professionals in real time or batch mode.

Regardless of its deployment location, the AI engine is operatively coupled to one or more inputs, including sensor data streams such as EEG signals, heart rate variability, skin temperature, respiration rate, and biometric identifiers. In some embodiments, the AI engine may also ingest contextual inputs such as time of day, historical session data, facial expression analysis, vocal tone recognition, and questionnaire responses. The engine then processes this multimodal data to infer the user's current neurological or physiological state.

Based on the inference result, the AI engine selects or dynamically generates a therapy protocol. The protocol may specify a target brain region or combination of regions (e.g., frontal, parietal, occipital lobes or cerebellum), suitable energy emitter portions or portals to be activated, the waveform type, stimulation frequency, intensity, duration, and emission pattern (e.g., simultaneous, alternate, random). In some embodiments, the AI engine may select or recommend therapy objectives such as focus improvement, stress relief, memory enhancement, emotional regulation, or relaxation, and configure corresponding emitter patterns.

The AI engine may incorporate machine learning techniques such as supervised learning, unsupervised clustering, or reinforcement learning. Supervised models may be trained on labeled data collected from previous therapy sessions and user-reported outcomes, allowing the engine to generalize therapeutic decisions across similar physiological states. Reinforcement learning agents may continuously adapt therapy configurations by maximizing a reward function linked to real-time sensor feedback and post-session user satisfaction scores. Unsupervised models may be used to detect novel user states or suggest experimental therapy routines for edge cases.

In an exemplary use case, the AI engine may analyze real-time HRV and EEG patterns to classify a user's current state as “pre-sleep anxious.” The engine may then retrieve historical data showing effective calming patterns for similar sessions and deploy a therapy protocol that initiates alternate pattern light stimulation at decreasing frequency, while also triggering soothing audio feedback. Over time, the AI engine learns from user-specific responses, adapting stimulation waveform shape, portal selection, and intensity modulation to optimize effectiveness.

In some embodiments, the AI engine may also coordinate with emotion recognition modules using facial detection or voice analysis to refine its inference models. The emotion module may classify the user's emotional state (e.g., sad, neutral, anxious, excited) and feed this information back into the AI engine to adjust therapeutic intent. The AI engine may then select more suitable emitter configurations or modify the auditory feedback being played to the user.

The AI engine may also support multi-user adaptability. For example, each user profile may be associated with a distinct model instance or with user-specific parameters in a shared model. Such configuration allows the AI engine to personalize therapy decisions based on individual characteristics, therapy histories, and response patterns. This capability may particularly be beneficial in environments where multiple users—such as household members or clinic patients—share a single apparatus.

In another embodiment, the AI engine may conduct a post-session analysis. Based on trends observed across sessions, such as improvements in sleep metrics or reductions in stress biomarkers, the engine may adjust future therapy routines. If the engine identifies diminishing returns on a previously effective stimulation protocol, it may initiate model branching or retraining to generate alternative protocols, thereby reducing habituation or tolerance.

To facilitate medical integration, the AI engine may also generate reports summarizing session performance, anomalies detected, and therapy evolution over time. These reports may be sent to caregivers, therapists, or clinicians for clinical decision-making. The AI engine may also implement compliance monitoring to detect skipped sessions, erratic usage patterns, or mismatched user input, which may be flagged for human intervention.

In embodiments where regulatory compliance or data privacy are mandated, the AI engine may encrypt and anonymize stored or transmitted data. Data partitioning techniques may be applied so that personal identifiers are decoupled from therapy history, allowing data to be used for research or training without compromising user confidentiality.

In some configurations, the AI engine may work in concert with cloud-based knowledge graphs or medical knowledge repositories to incorporate evidence-based therapeutic models, clinical trial results, or diagnostic protocols. This hybrid intelligence approach may allow the system to reconcile user-specific data with broader treatment standards.

Accordingly, the AI engine described herein enables the neurostimulation apparatus to function as a self-learning, adaptive, and intelligent therapy system capable of operating autonomously or under supervision, dynamically customizing therapy sessions based on real-time data, learned user behavior, and external medical inputs.

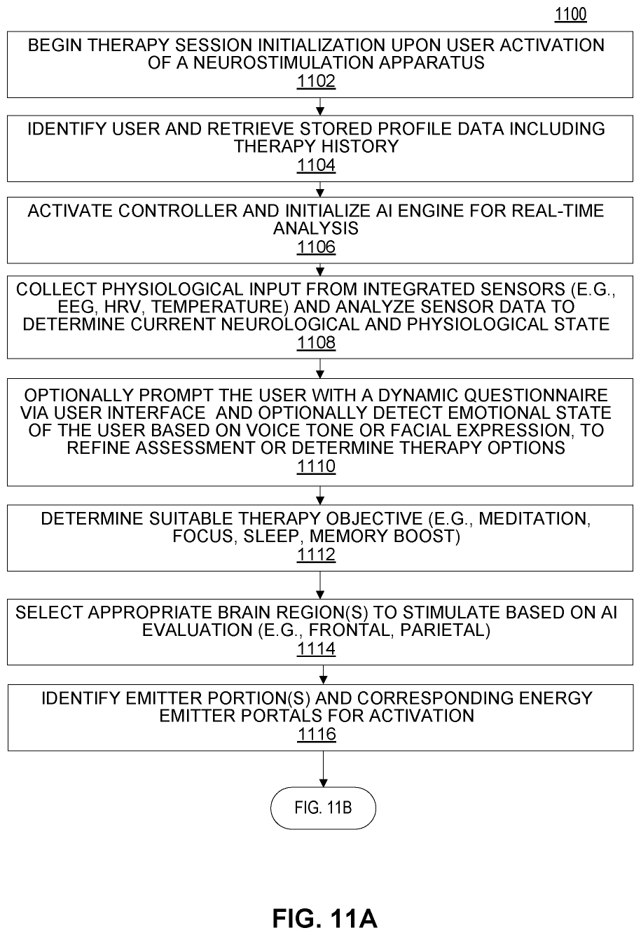

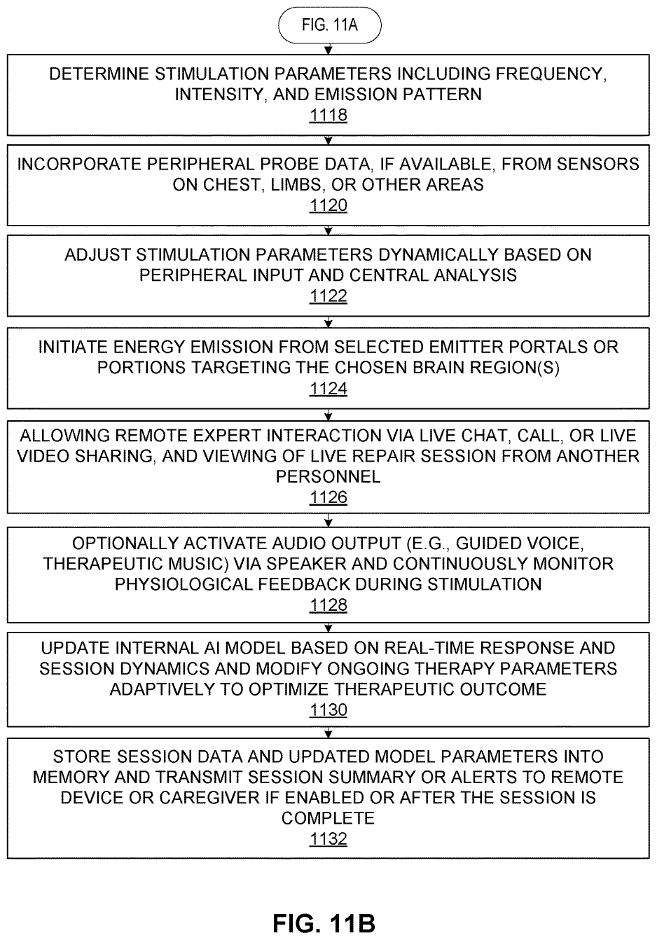

Referring now to FIGS. 11A-11B, a flowchart 1100 is illustrated depicting an exemplary method for adaptive neurostimulation therapy using a wearable neurostimulation apparatus (e.g., 100), in accordance with various embodiments of the present invention. The method begins with initializing the session upon user activation and proceeds through a series of intelligent, sensor-driven, and AI-supported steps to personalize and dynamically modulate therapy.

Referring now to FIG. 11A, a method step 1102 begins with therapy session initialization upon user activation of a neurostimulation apparatus. This activation may occur via physical input from a user interface on the apparatus, such as a power button, or through remote initiation using a connected user device. The apparatus may be used in various contexts including in-clinic, at-home personal care, or even within guided therapy programs where a preset schedule of stimulation sessions is followed. The initiation may also trigger a self-diagnostic sequence within the system to verify functional readiness, hardware integrity, and sensor calibration before beginning stimulation.

At step 1102, the therapy session initialization begins upon user activation of the neurostimulation apparatus. The activation may involve the user pressing a physical button on the apparatus, initiating a command through a connected user device, or issuing a voice-based instruction if a microphone interface is included. This initiation event signals the system to prepare all hardware and software subsystems for therapy delivery. In some embodiments, this may also involve powering up emitter circuits, preparing sensors for data acquisition, and activating memory buffers for logging data. The session start may trigger internal timestamps and indexing for session tracking.

At step 1104, the system identifies the user and retrieves stored profile data including therapy history. User identification may occur through biometric sensors embedded in the device, such as fingerprint, facial recognition, or voice signature. In other embodiments, identification may be based on previously paired user devices via wired or wireless connection. Once the user is recognized, the controller retrieves associated data such as prior therapy types used, frequency preferences, response patterns, and physiological baseline data. This historical information allows the system to tailor the upcoming session in a personalized and adaptive manner.

At step 1106, the controller is activated and the AI engine within the controller is initialized for real-time analysis. This may include loading pre-trained models into the processor and preparing inference routines to begin analyzing sensor data as it arrives. The AI engine may include neural network architectures trained on cognitive response datasets, stress-level detection frameworks, and brain state classification models. The activation step may also include verifying the integrity of sensor circuits, initiating baseline calibration if needed, and preparing memory and buffer allocations for upcoming processing tasks.

At step 1108, physiological input is collected from integrated sensors, which may include electroencephalography (EEG) for monitoring electrical brain activity, heart rate variability (HRV) for autonomic nervous system analysis, and skin temperature for detecting relaxation or stress levels. The AI engine receives this data and performs pattern recognition or statistical correlation against stored profiles or normative datasets. For example, high beta wave dominance in EEG may correlate with stress or heightened alertness, whereas increased alpha activity may suggest calmness. The analysis helps derive the current neurological and physiological state of the user.

At step 1110, the system may optionally prompt the user with a dynamic questionnaire through a user interface, such as a touch display or mobile device. The questionnaire may include questions like “Are you feeling anxious today?”, “How well did you sleep last night?”, or “What is your current focus level?” The answers may be processed using natural language understanding models or simple rule-based logic. Alternatively, or in combination, the system may use passive inputs such as tone of voice or facial expressions captured via a connected camera or microphone to infer the emotional state of the user. A user exhibiting a lowered gaze, flattened affect, and slow speech rate may be inferred to be in a depressive state, thus prompting the system to adjust therapeutic strategies accordingly.

At step 1112, based on the analysis of sensor data, questionnaire responses, and emotional detection, the system determines a suitable therapy objective. Objectives may include but are not limited to relaxation, meditation, focus enhancement, sleep induction, memory improvement, or energy boosting. For example, if the user is found to be in a stressed and unfocused state, the system may select a dual-targeted therapy to both reduce stress and improve attention span. This step may include selecting a goal and aligning the subsequent stimulation process toward that desired outcome.

At step 1114, the AI engine selects the appropriate brain regions to stimulate based on the identified therapy objective. For example, if the objective is to enhance working memory, the stimulation may be directed to the dorsolateral prefrontal cortex. If the goal is visual enhancement or relaxation, the occipital or parietal lobes may be targeted. The selection process may rely on a mapping database correlating functional brain regions with therapy objectives. The AI engine uses real-time assessment, past response data, and physiological feedback to select one or more brain regions for stimulation.

At step 1116, the system identifies the emitter portions and their corresponding energy emitter portals within the neurostimulation apparatus that align with the selected brain regions. Each emitter portion may include an array of light emitters, vibratory actuators, or acoustic transducers, each associated with a unique portal on the apparatus surface. The selected emitters are aligned geometrically with the user's skull anatomy to direct stimulation energy—such as infrared, near-infrared, or red light-toward the targeted brain regions. The control unit may also identify whether the emitter activation should occur simultaneously, sequentially, or in alternating modes across the selected portions.

At step 1118, the system determines stimulation parameters including frequency, intensity, and emission pattern. The frequency may range between 1 Hz to 500 Hz depending on the therapeutic goal, such as alpha stimulation for relaxation or gamma-range for focus. The intensity may be calibrated based on past user response or real-time feedback. The emission pattern can be continuous, pulsed, alternating, or randomized, depending on whether the objective is stimulation, entrainment, or modulation. These parameters are transmitted to emitter control modules for execution.

At step 1120, the system incorporates peripheral probe data, if available, from sensors attached to the user's chest, limbs, or other areas. These probes may measure heart rate, skin conductance, muscle tension, blood oxygen levels, or other physiological markers. The probe data offers contextual insight about systemic physiological conditions and enhances central decision-making by correlating peripheral metrics with brain activity trends.

At step 1122, the system dynamically adjusts stimulation parameters based on both central neurological data and peripheral physiological feedback. For example, if HRV data from a chest probe indicates rising stress levels despite the current stimulation setting, the system may lower frequency and increase light intensity to induce a calming effect. Alternatively, increasing limb muscle activity may suggest motor stimulation response, leading the system to maintain or extend therapy for motor rehabilitation.

At step 1124, the system initiates energy emission from selected emitter portals or emitter portions, targeting the chosen brain regions as identified in prior steps. Emitters may operate in patterns prescribed by the controller, delivering non-invasive photonic, vibratory, or sonic stimulation to the scalp. These emissions penetrate the skull to engage neuronal populations underlying the surface. Depending on the hardware configuration, emission onset may be phased, staggered, or grouped to avoid overstimulation or to leverage inter-hemispheric synchrony.

At step 1126, the system optionally enables remote expert interaction. Through secure communication protocols, a therapist or clinical expert can monitor the live session, initiate a chat, or join via a video stream. The expert may assist with parameter adjustments, provide motivational support, or guide users through cognitive-behavioral interventions. This feature is particularly useful in remote therapy, rehabilitation for neurological conditions, or for pediatric users.

At step 1128, the system optionally activates audio output through a speaker embedded in the apparatus. This may include playback of guided meditation, binaural beats, neuro-acoustic tones, or verbal cues. The content may be dynamically selected based on therapy objectives. The system continuously monitors real-time sensor data to assess how the user is responding to the combined audio and stimulation, allowing adaptive modulation of volume or content type.

At step 1130, the internal AI model is updated based on real-time user response, session dynamics, and recorded changes in physiological and neurological data. This model refinement allows the apparatus to improve session outcomes over time, adapting to user-specific neural patterns. If a user repeatedly responds well to a certain configuration, the system promotes that pattern for future sessions. This adaptive learning enables a closed-loop therapy mechanism.

At step 1132, the system stores all relevant session data, including stimulation parameters, physiological response logs, and model adjustments into memory. If the system is enabled for connectivity, the data may also be transmitted to a remote server, caregiver, or medical professional for analysis, archival, or long-term tracking. Alerts may be generated for abnormal response trends, missed sessions, or recommendations for therapy modification. Upon storing or transmitting the session summary, the method concludes until the next activation cycle is initiated.

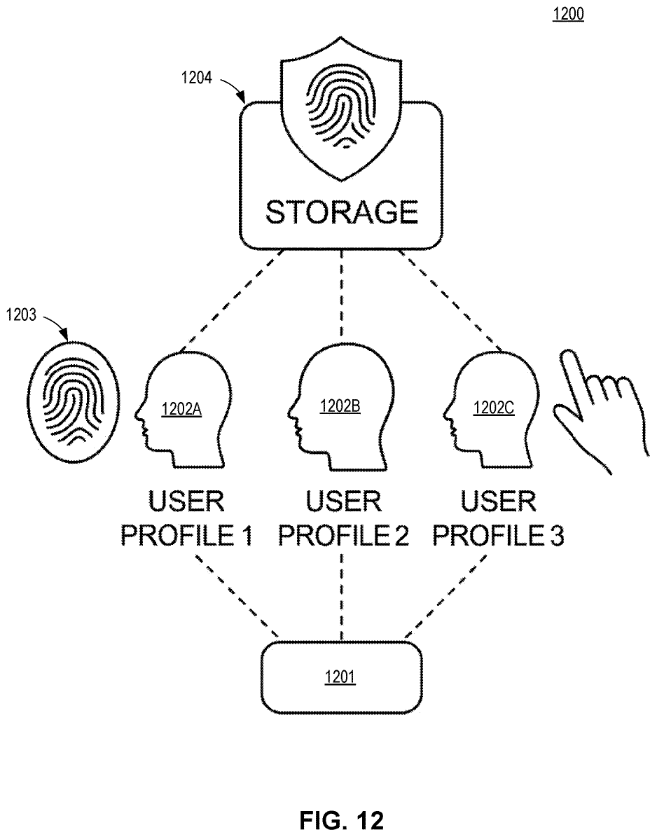

Referring now to FIG. 12, an exemplary multi-user profile management system 1200 is illustrated for a neurostimulation apparatus 1201. The system 1200 may be configured to support a plurality of distinct user profiles for different individuals utilizing the same neurostimulation apparatus 1201 in a household, clinic, or shared-care setting. The figure depicts three distinct user profiles, respectively denoted as user profile 1, user profile 2, and user profile 3, associated with respective users 1202A, 1202B, and 1202C. These user profiles may be uniquely identified, stored, accessed, and switched between, depending upon the specific user interacting with the apparatus 1201.

Each user profile (e.g., user profiles 1-3) may be configured to store personalized neurostimulation parameters, historical therapy data, therapy preferences, sensor calibration data, and AI-derived recommendations. The neurostimulation apparatus 1201, upon activation, accesses a profile recognition subsystem which determines the currently interacting user among the set of possible users, such as 1202A, 1202B, or 1202C. In an embodiment, this determination may be made using biometric input, such as a voice signature, facial recognition, or fingerprint authentication.

The biometric recognition functionality may be achieved through various sensor integrations. For example, the apparatus 1201 may be configured with an onboard microphone capable of capturing the user's voice. The AI engine within the apparatus 1201 may be trained to recognize unique voice patterns, pitch, and modulation characteristics associated with respective users. In some embodiments, once the user 1202A speaks a predefined activation phrase or interacts with the device, the apparatus 1201 identifies the user by comparing the input with stored voice signatures associated with the plurality of stored profiles.

Alternatively, or additionally, the system 1200 may utilize facial recognition technologies via an externally connected user device such as a smartphone or tablet. Such user devices may operate a dedicated application, which activates the front-facing camera, captures facial imagery of the user (e.g., 1202A), and then authenticates this data against stored biometric templates. In this embodiment, the selected user profile may be transmitted to the apparatus 1201 via a wired or wireless communication link for therapy customization.

The fingerprint identification capability, represented by reference numeral 1203 in FIG. 12, may be embedded within the apparatus 1201, or may be implemented on a connected user device or an auxiliary accessory. The user 1202C, for example, may place a finger on a sensor module, and the system 1200 uses a stored encrypted mapping of fingerprint identifiers to determine that the user corresponds to user profile 3. The profile identification enables the apparatus 1201 to immediately load specific stimulation configurations, therapy schedules, and prior history tied to that profile.

In another embodiment, a manual selection mode may be supported, wherein the user 1202B manually selects their identity from a list displayed on a connected interface, such as a mobile phone, tablet, or onboard screen. The manual interface may be configured with touchscreen, gesture-based, or button-based inputs. The selection of a user profile may require further authentication, such as entry of a passcode, drawing of a gesture pattern, or confirming a push notification sent to a registered device.

The system 1200 includes a secure storage module 1204, which may reside locally in the apparatus 1201, remotely on a server, or in a hybrid configuration. The storage module 1204 may be responsible for storing each user's therapy parameters, including stimulation history, feedback logs, adaptive AI models, and control parameters. Security of the stored profiles may be maintained using encryption, digital signatures, and access control lists that restrict unauthorized access.

In some embodiments, the storage module 1204 also stores AI-generated recommendations specific to each user. For example, user 1202A may have a neurostimulation profile adapted for cognitive focus with emission patterns in the frontal cortex region, while user 1202C may require a calming stimulation mode targeting occipital lobes for sleep enhancement. Each therapy recommendation may be individualized based on prior session response and sensor feedback collected over time.

The apparatus 1201 uses this user-specific information to tailor every aspect of the session. Once a user is authenticated and the profile is loaded, the stimulation session parameters such as intensity, pulse frequency, duration, emitter selection, and dynamic response models are customized according to the stored preferences and learned data. This eliminates the need for reconfiguration by the user every time and allows seamless transition between users.

The system 1200 supports remote configuration and synchronization of user profiles. If therapy is monitored or updated by a clinician or a caregiver, they may push updates to individual user profiles stored in 1204. For example, a therapist may adjust the stimulation range for user 1202B based on weekly performance reports and sensor analytics, and such adjustments are immediately available upon the next activation by that user.

Each profile, such as those for users 1202A, 1202B, and 1202C, maintains a separate therapy history. This may include session timestamps, used emitter parameters, user feedback, detected biometrics like HRV (heart rate variability), EEG signals, voice tone variations, and facial affective state. These data points are used by the AI engine to continuously improve the personalized stimulation model for each user.

In another embodiment, user profiles stored in storage module 1204 may be linked to cloud accounts or user devices for backup, synchronization across multiple apparatus units, or for long-term progress monitoring. For example, user 1202A may begin a therapy session at home on a personal neurostimulation unit and continue the session at a clinic using a shared unit by accessing their cloud-synced profile.

In a multi-user environment such as a family or rehabilitation center, the system 1200 allows for frictionless switching between users. When user 1202B ends a session and user 1202C takes over, the apparatus 1201 can reset emitter configurations, adapt the internal models, and fetch appropriate session data from storage module 1204 without manual intervention. This capability supports shared hardware usage while preserving individualized therapy.

The apparatus 1201 may also be configured with contextual detection to identify the user profile passively, for example by recognizing habitual usage patterns, device handling gestures, or ambient sounds. In such a passive recognition scenario, the controller may predict the active user and verify it through a secondary biometric input.

In some embodiments, the AI engine within the apparatus 1201 or in connection with storage module 1204 maintains usage analytics across all profiles. These analytics may be useful for determining which users are benefitting from therapy, who requires reassessment, and whether profiles need to be modified or merged based on overlapping patterns.

The system also supports privacy compliance mechanisms. Each user profile stored in storage 1204 can be encrypted with keys unique to each user or protected with biometric encryption schemes such that profile data can only be accessed after correct authentication.

Additionally, different access rights may be assigned to each user profile. A primary caregiver or administrator profile can view or modify all other profiles, whereas regular users like 1202B can only view or modify their own data. The system can incorporate parental controls, supervision logs, and usage constraints to accommodate juvenile or vulnerable users.

Through integration with the broader neurostimulation system, each user profile can contain preset modules like “Focus Boost,” “Deep Calm,” or “Memory Enhancement” and the respective energy portal activation sequences and session times linked to them. Upon user 1202C's selection of a predefined therapy mode, the system uses their profile settings to configure stimulation on the fly. In another embodiment, profile switching may be voice-activated. For example, a command like “Activate my profile” spoken by user 1202A triggers the system to authenticate the voice, compare it to stored biometric profiles, and load user profile 1 accordingly, allowing a seamless, touch-free interaction.

Further, the apparatus 1201 may log switching events, profile access times, and failed authentication attempts for security auditing and maintenance of system integrity. This log data can be retrieved and analyzed to detect any unauthorized access patterns. The user profiles may also incorporate mood-state history captured through facial or voice emotion detection, which aids in assessing therapy effectiveness longitudinally. For example, user 1202B's frequent reports of improved mood post-session can be tagged in the profile and used to reinforce similar stimulation models in the future.

In other implementations, shared therapy configurations across users may be supported. For example, if multiple users (e.g., 1202A and 1202B) show improvement with similar temporal lobe activation models, the AI system may recommend these shared parameters during therapy model updates, while still maintaining user-level customizations. In some scenarios, the storage module 1204 also holds session media such as guided meditation tracks, auditory stimulation files, and neurofeedback training modules tailored to individual user responses.

Users may be allowed to clone their profiles to new apparatuses or share their profiles temporarily for session testing or collaborative therapy with medical professionals. Such profile portability can enhance therapy accessibility across different devices.

The system 1200 may also implement scheduled profile activations, allowing users to preset therapy times where the device automatically starts a session using their stored profile upon recognition. A caregiver, therapist, or administrator may be allowed to generate new user profiles via a secure interface, where initial therapy settings and biometric registration steps are performed. These profiles are then assigned to individuals such as 1202A or 1202C through biometric linking.

User profiles may also store consent metadata, session feedback ratings, and compliance data such as daily usage statistics and therapy adherence graphs, all of which are available for review by authorized parties. The apparatus 1201 may additionally support switching between active and passive profiles. For example, an observer profile may be used during demonstration without triggering any actual energy stimulation.

Finally, the multi-user system 1200, as illustrated in FIG. 12, allows a single neurostimulation apparatus 1201 to serve multiple individuals with full personalization, privacy, adaptability, and extensibility, enabling scalable therapeutic solutions for both household and institutional deployments.

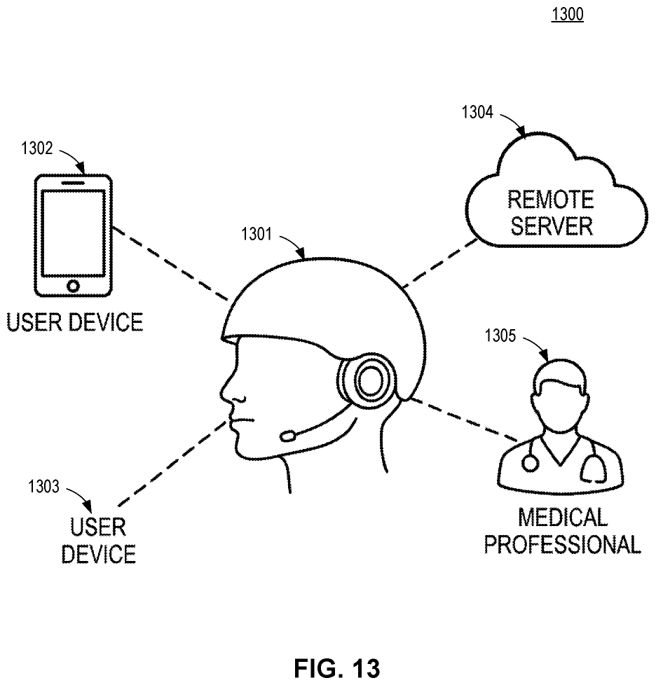

Referring now to FIG. 13, an exemplary system architecture 1300 is illustrated, which depicts the interconnectivity between a neurostimulation apparatus 1301 and various external entities including user devices 1302 and 1303, a remote server 1304, and a medical professional 1305. The neurostimulation apparatus 1301 may be configured with wired or wireless communication capabilities to facilitate real-time and/or deferred interaction with each of the aforementioned entities, in accordance with various embodiments of the present invention.

The neurostimulation apparatus 1301 may include communication circuitry embedded within its housing to support short-range wireless standards such as Bluetooth, Wi-Fi, ZigBee, or low-energy protocols, as well as long-range connectivity such as cellular, LoRa, or satellite-based links. Alternatively, the communication may occur through physical interfaces such as USB-C, micro-USB, or proprietary ports. The apparatus 1301 may switch between communication modes based on the operational context, user preferences, battery conservation strategies, or environmental electromagnetic conditions.

User device 1302, as shown, may be a smartphone, tablet, or wearable device that provides a user interface for controlling the therapy session parameters, monitoring real-time brain activity or physiological responses, and adjusting configurations on-the-fly. In some embodiments, device 1302 may include a dedicated application that allows users to choose therapy modes, view history, engage in questionnaires, or review sensor feedback during or after a session. Device 1302 may also store encrypted profile data for identifying the user and loading personalized therapy settings prior to activation.

In some scenarios, user device 1303 may function independently or complementarily to device 1302. For example, user device 1303 may be installed at a stationary location such as a home docking station, clinic kiosk, or bedside controller. This device may maintain a higher processing capacity or persistent data storage to retain long-term records of therapy outcomes, device usage logs, and firmware updates. In some configurations, user device 1303 may also be utilized by caregivers or supervising personnel to administer or supervise the therapy routines remotely or locally.

Remote server 1304 is depicted as a cloud-based infrastructure facilitating secure data exchange between the apparatus 1301 and remote monitoring systems. The server 1304 may store large volumes of anonymized and/or individualized data including therapy session records, biometric input logs, environmental conditions, and AI evaluation metrics. Through this storage, the system may support deep-learning frameworks that continuously refine the AI models embedded in the neurostimulation apparatus 1301.

The connection between the apparatus 1301 and remote server 1304 may be selectively enabled before or after the therapy session to reduce potential exposure to electromagnetic radiation during therapy. In some embodiments, the apparatus 1301 may enter an “offline” mode once therapy is initialized, only resuming wireless communication after the therapy ends. This design may be beneficial in applications involving sensitive neurological states, such as meditation, sleep aid, or therapeutic intervention for individuals with neurodegenerative disorders or electromagnetic hypersensitivity.

Medical professional 1305 is shown as another endpoint within the system 1300, receiving data from the neurostimulation apparatus 1301 either directly or via the remote server 1304. In some configurations, the professional 1305 may have access to a secure dashboard providing real-time session telemetry, longitudinal progress tracking, alert notifications, and session override functionalities. The apparatus 1301 may be preconfigured to trigger alerts or prompt review if abnormal patterns are detected in EEG, HRV, respiration rate, or user behavior, as determined through integrated sensors.

The communication between the medical professional 1305 and the apparatus 1301 may support interventions such as live therapy adjustments, remote diagnostics, or issuing emergency alerts if the patient experiences distress. For example, the professional may remotely activate a “pause” command or modify intensity levels based on patient reactions. The system may also support audiovisual conferencing features for consultation before or after therapy sessions.

The communication diagram 1300 also highlights the bidirectional flow of data between all entities. The apparatus 1301 may transmit performance logs, physiological feedback, and error codes, while receiving updated therapy models, personalized presets, and firmware updates. Such continuous data flow supports closed-loop AI refinement, enabling the apparatus to deliver increasingly precise and adaptive therapies.

In some embodiments, a secure authentication protocol may be employed at each communication endpoint. For example, the apparatus 1301 may require dual-factor authentication from a user device 1302 and biometric confirmation before initiating session sync with remote server 1304 or medical professional 1305. Such an arrangement increases compliance with medical data protection laws including HIPAA, GDPR, and local jurisdictional requirements.

The system 1300 may also enable scheduled session synchronization, wherein therapy sessions stored within the apparatus 1301 are periodically uploaded to the remote server 1304 during low-network-usage hours. Alternatively, session data may be transferred manually via user device 1302 acting as a relay, especially in low-connectivity environments or rural regions.

In certain implementations, the user device 1302 or 1303 may also act as a remote training agent, delivering AI updates to the apparatus 1301 after model recalibration on the server 1304. This offloads heavy computation from the wearable to the cloud or the local device while allowing personalization and performance enhancements.

The connection topology shown in FIG. 13 supports modularity in deployment. For example, the system may operate in standalone mode with only the apparatus 1301 and a user device 1302, or in enterprise mode involving all four entities interconnected via a secure network. Such flexibility may be advantageous for clinical trials, multi-user household settings, or institutional deployments.

In some embodiments, the apparatus 1301 may selectively disable uplink or downlink paths during therapy execution, with configuration governed by a session-specific profile. For example, during high-intensity sessions targeting memory enhancement, the AI engine within the controller may automatically mute all external communication.

Additionally, the medical professional 1305 may initiate asynchronous review of patient data, generating a report or modified prescription plan that is pushed to user device 1302 for implementation during the next session. Such a framework supports distributed care and long-term engagement.

In other embodiments, user devices 1302 or 1303 may feature parent/guardian control interfaces in pediatric or geriatric applications. These interfaces may include session timers, access restrictions, and caregiver notifications, offering an additional layer of user safety and compliance.

The user interface of device 1302 may include a graphical representation of the neural regions targeted, displaying session progress, real-time vitals, and response indicators such as color-coded feedback loops. User feedback captured through voice or screen interactions may be processed and transmitted to the server 1304 or professional 1305.

As depicted in FIG. 13, the apparatus 1301 lies at the center of a distributed intelligence system 1300, with each peripheral entity adding context, personalization, safety, or oversight. The design accommodates both real-time interaction and delayed batch communication, maximizing utility across scenarios ranging from home wellness to regulated clinical therapy.

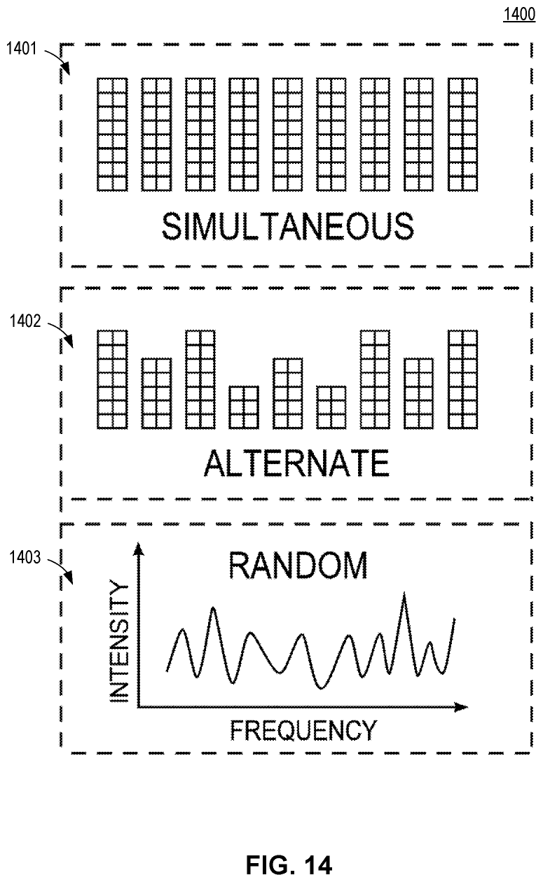

Referring now to FIG. 14, an exemplary schematic diagram 1400 is depicted, illustrating three distinct energy emission pattern variants usable by a neurostimulation apparatus, namely simultaneous emission 1401, alternate emission 1402, and random emission 1403. Each of these stimulation modes governs the temporal and spatial activation of a plurality of emitter portals disposed within one or more emitter portions of the neurostimulation apparatus. These variations enable flexible and dynamically adaptive therapy strategies, tailored to individual user response patterns, brain region targeting requirements, or physician-programmed protocols, in accordance with various embodiments of the present invention.

In the simultaneous stimulation mode 1401, each vertical bar illustrated within the boxed segment represents an individual emitter portal or a grouped emitter array configured to activate in unison across a predetermined time interval. This synchronous activation pattern is designed to deliver uniform stimulation energy—such as photonic, thermal, or vibrational impulses—across multiple anatomical regions concurrently. For example, simultaneous emission may be employed during generalized relaxation therapy wherein uniform energy dispersal over frontal, parietal, and occipital regions is desirable to induce whole-brain calming.

In the illustrated depiction of simultaneous emission 1401, the rows of uniformly sized vertical bars indicate that all relevant emitter portals are activated concurrently and maintain consistent amplitude (emission intensity) over successive intervals. The uniform height and spacing of the bars indicate equivalent power output and synchronized firing duration. This configuration may be achieved by programming the controller of the neurostimulation apparatus to activate all targeted emitter portals with identical pulse duration, amplitude, and waveform characteristics.

The alternate emission pattern 1402, by contrast, demonstrates staggered or interleaved activation among different emitter portals or arrays. Each bar again represents an individual emitter or an emitter group, and the variations in bar heights reflect modulation of intensity, while the staggered positions reflect non-overlapping activation phases across the emitter network. Alternate emission patterns are advantageous when therapeutic targeting of multiple brain areas is intended without concurrent stimulation to avoid potential signal interference or overstimulation.

An embodiment utilizing alternate emission 1402 may involve first stimulating the prefrontal cortex, then shifting sequentially to the parietal lobe, and then the occipital region. This rotating stimulation sequence can be utilized to deliver stimulation with controlled pacing that mimics natural neural oscillations or to accommodate specific neurological conditions such as attention disorders, where staggered pulses are more effective than full-area stimulation.

Each bar in the alternate emission depiction 1402 may represent an emitter portal activated during a discrete time slice, and the varying bar heights reflect differential intensity values. The system may assign emitter portals to a timed activation queue wherein each portal may be energized for a fixed duration and then transitioned to the next portal in the schedule. Such a method may be advantageous in reducing energy load, thermal buildup, or potential desensitization of skin or neural tissue.

Moving to the random emission pattern 1403, a graphical representation is shown in the form of a continuous intensity-versus-frequency waveform. Unlike the structured bar patterns of the earlier modes, random emission 1403 introduces stochastic or semi-stochastic temporal variation in activation intervals, emitter selection, and intensity levels. This method may be useful for mimicking natural neurological variability or introducing novelty into stimulus exposure for conditions like cognitive flexibility training or neuroplasticity enhancement.

In this context, random emission 1403 may involve algorithmic or AI-generated output patterns whereby the selection of emitters, intensity, and pulse duration is determined on-the-fly in response to sensor feedback or as part of a pre-programmed pseudo-random therapeutic protocol. For example, to activate associative learning regions of the brain, the system may randomly stimulate combinations of frontal and temporal lobes using differing light wavelengths or intensities.

Each point on the waveform in the random emission graph 1403 may correspond to a single energy burst from one or more emitters, with the Y-axis representing pulse intensity and the X-axis representing chronological progression. This emission pattern may also be defined by constraints such as avoiding consecutive stimulation to the same emitter or respecting a recovery interval between pulses, thereby safeguarding user comfort and system longevity.

In some embodiments, the AI engine integrated within the controller of the apparatus may dynamically switch between simultaneous, alternate, and random emission patterns during a session depending on the user's physiological responses, voice cues, or pre-therapy questionnaire outcomes. This adaptive modulation allows personalized stimulation trajectories that evolve in real time.

In alternate embodiments, random emission 1403 may be used for desensitization or tolerance mitigation protocols, wherein the apparatus injects variability to reduce the predictability of stimulus reception, which is theorized to enhance sustained user engagement and long-term therapeutic outcomes.

In yet another embodiment, a hybrid mode may be configured wherein simultaneous stimulation is used during initiation, followed by alternating pulses for mid-session control, and concluded with random bursts to trigger reflexive brain states. This sequencing may be managed by the therapy protocol defined in the user profile or controlled remotely by a medical professional.

As shown in FIG. 14, the system may also associate each emitter portal with a positional identifier and a programmable pattern slot, thereby enabling reproducibility of spatial patterns, whether for rehabilitation targeting specific neural circuits or for cyclic modulation across therapy sessions.

The use of these emission patterns as depicted in segments 1401, 1402, and 1403 may further be encoded into digital session logs, allowing for future AI training or retrospective clinical analysis. For example, the session logs may contain data mapping user response metrics to the corresponding emission pattern in use during that timeframe.

Additionally, each emission pattern may correspond to different waveform structures such as sinusoidal, pulsed square, or custom wavelets, depending on the stimulation goal. For example, in cognitive alertness stimulation, simultaneous mode with rapid square pulses may be preferred, while for meditative calming, alternate low-frequency sinusoidal patterns may be programmed.

In some embodiments, these emission patterns may not only be determined per session but may also be changed dynamically in real-time based on physiological feedback from onboard sensors such as EEG, heart rate variability, or temperature sensors. This makes the stimulation reactive and customized to the user's neurological state in real time.

To implement the emission patterns shown in FIG. 14, each emitter portal may be controlled by a software-controlled matrix driver that supports independent control of current, voltage, and timing across multiple channels. The controller may apply lookup tables or AI-generated outputs to decide the activation schema per pattern. The alternate and random stimulation strategies may also be leveraged in group therapy or dual-user modes wherein synchronized but phase-shifted therapy is provided across multiple users using identical or slightly offset patterns to support collective therapeutic environments.

As another use case, random emission patterns (e.g., 1403) may be programmed to simulate sensory environments such as forest sounds or ocean waves, wherein rhythm and energy pulses replicate natural irregularities, thus enhancing immersion and engagement during therapy. In still other embodiments, alternate stimulation (e.g., 1402) may be used to manage localized energy concentration, where only a select few emitter portals are activated in each cycle, thereby preventing overheating or energy overload on the user's skin or scalp surface.

The bar representations in FIG. 14 are also symbolic of modular timing blocks, meaning that each bar may correspond to a 100 ms or 200 ms emission window during which a portal is active, which may vary depending on the user's condition, age, or sensitivity threshold.

These emission pattern diagrams may be incorporated in the user interface for medical professionals or users to manually select, review historical patterns, or override AI-based recommendations when necessary, such as during manual rehabilitation routines or guided learning protocols.

In further embodiments, users may use voice commands to select a preferred stimulation pattern (e.g., “Switch to alternate pulse mode”), enabling ease of control without physical interaction with a mobile device or control terminal, particularly beneficial for users with mobility challenges. In configurations where visual confirmation is important, the emission pattern may be accompanied by LED indicators embedded in the apparatus that blink in real time according to the pattern being emitted, thereby creating biofeedback loops with the user.

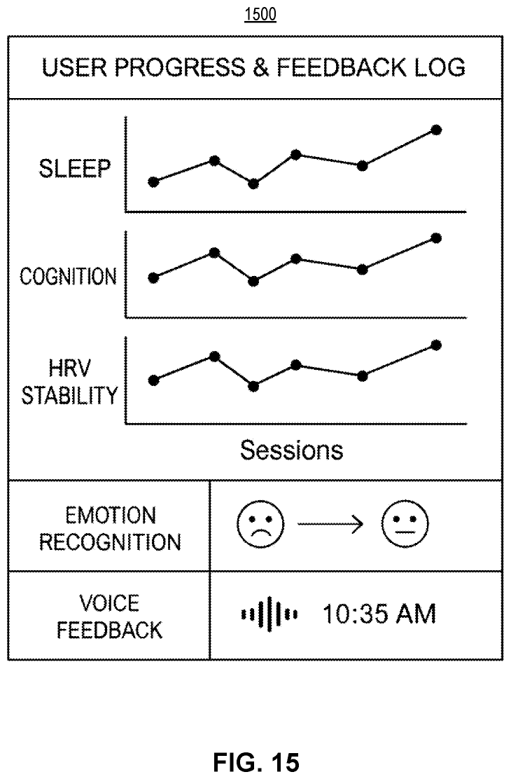

Referring now to FIG. 15, an exemplary user progress and feedback logging interface 1500 is illustrated, wherein the system presents a multi-dimensional report interface capable of displaying session-by-session therapeutic performance data, alongside real-time emotional and vocal feedback analysis. In accordance with various embodiments of the present invention, the user progress tracking module depicted in FIG. 15 is configured to provide a detailed, session-wise graphical output for core wellness parameters including sleep, cognition, and heart rate variability (HRV) stability, while concurrently capturing emotional transitions and timestamped voice input that may be used to guide adaptive therapy control, retrospective evaluation, or professional review.

As illustrated, the upper portion of interface 1500 may include graphical plots corresponding to user-specific longitudinal metrics spanning multiple therapy sessions. A first graph labeled “SLEEP” tracks sleep-related performance parameters such as duration of sleep cycles, onset latency, frequency of awakenings, or self-reported restfulness, as may be extracted from wearable sensors or user surveys. Each plotted data point may represent a quantified sleep index calculated by combining biometric indicators and subjective input, where upward trends are indicative of improving sleep quality.

Below the sleep graph, a second metric labeled “COGNITION” depicts temporal fluctuations in cognitive functioning as experienced by the user across the same series of sessions. In some embodiments, this metric may be derived from real-time psychometric assessments such as memory recall tasks, attention span evaluations, or reaction time tests conducted via a connected user device. Alternatively, cognitive assessment scores may be self-reported by the user or interpreted using external tools integrated with the neurostimulation apparatus, such as vision-based eye tracking systems or EEG attention markers.

A third chart labeled “HRV STABILITY” is presented in the lower portion of the graph cluster. HRV stability reflects the user's cardiac autonomic response consistency across time and serves as a reliable proxy for stress adaptation and neurological balance. In certain embodiments, HRV measurements may be taken via peripheral probes, such as chest-mounted sensors or integrated earlobe pulse detection, with the HRV data streamed to the system's memory and plotted in real-time or during post-session analytics.

Each of the line charts presented in interface 1500 is composed of discrete data points connected sequentially, wherein each point corresponds to an individual neurostimulation session. The spacing between the points may indicate uniform time intervals, or alternatively, the time axis may be calibrated to reflect actual temporal gaps between sessions, depending on implementation preferences. The vertical axis in each chart may reflect either raw values or normalized scores for comparative visual evaluation.

The lower segment of the interface 1500 may include two additional blocks: “EMOTION RECOGNITION” and “VOICE FEEDBACK.” The “EMOTION RECOGNITION” segment visually depicts a before-and-after transition between emotional states, indicated by stylized icons representing a sad face and a neutral or happy face. In some embodiments, the system is configured to detect user emotion pre- and post-therapy using facial expression analysis, vocal tone analysis, or a fusion of multiple biometric cues. This visual representation helps the user and professional caregivers quickly grasp the emotional efficacy of the recent session.

The “VOICE FEEDBACK” section in the bottom-left corner of FIG. 15 presents a voice waveform icon alongside a timestamp (e.g., 10:35 AM). This element indicates that the system has logged a verbal user response or comment during or after the session. In certain embodiments, the timestamp corresponds to the moment the voice was recorded, and the waveform representation may link to an audio file that can be played back for review. This voice data may be parsed by onboard natural language processing models to extract sentiment, intent, or self-report feedback, and this derived content may be tagged to the associated session record.

In some embodiments, the voice feedback data may be transcribed automatically, with summary keywords or emotion tags being appended to the session log. For example, if the user utters phrases like “I feel more alert now” or “Still a bit anxious,” the system may tag the session accordingly with descriptors such as “alertness boost” or “anxiety residual,” enhancing the session context for retrospective analysis or AI-based therapy refinement.

The emotion recognition block and the voice feedback block may both contribute real-time inputs to the system's AI engine, which continuously adapts stimulation strategies based on aggregated user data. These feedback inputs may influence the next session's configuration by adjusting parameters such as stimulation intensity, emitter selection, duration, or associated audio playback content.

In further embodiments, the interface 1500 may be integrated into a user device application or web-based dashboard, allowing remote access for caregivers or medical professionals. Permissions and encryption protocols may be applied to maintain user privacy, particularly when emotion logs or voice files are transmitted across devices or stored in a cloud-based repository.

The progress graphs may also be dynamically scaled to accommodate additional metrics as needed. For example, metrics for anxiety reduction, motor responsiveness, pain index, or memory performance may be added, depending on the therapeutic objectives and hardware capabilities of the deployed neurostimulation apparatus.

In some implementations, the plotting interface may include color-coded trend lines or background shading to signify thresholds of performance. For example, green bands may represent acceptable ranges of HRV stability, while red may indicate abnormal fluctuations, alerting the system to adjust the therapy algorithm or prompt user intervention.

The progress interface 1500 may also include interactive controls enabling the user to tap or click on a data point to view detailed session summaries, including therapy parameters used (e.g., emitter portals activated, stimulation mode, waveform type) and user-reported responses. This functionality enhances transparency and user engagement.

In some embodiments, the session data logged in interface 1500 may be used to train or refine the AI engine of the apparatus. By correlating user improvement trends with specific stimulation configurations, the AI model may improve its predictive power in recommending optimal therapy settings for future sessions.

The logging interface 1500 may also serve as a compliance function. Users may be required to engage with their progress charts after each session, promoting self-awareness and adherence. Additionally, this interface may be audited by care providers to validate that prescribed therapy durations and intensities are being followed.

The emotion recognition module, shown in FIG. 15, may leverage advanced emotion classification networks that incorporate multiple modalities—facial expression detection via camera input, heart rate variability signals, and speech patterns—to generate robust predictions of emotional state transitions. These predictions may then be visualized for intuitive review.

In yet another embodiment, the progress interface may include a journal or annotation field where users can manually input contextual notes—such as medication intake, physical activity, or external stressors—that may influence session results. These notes may later be correlated with progress curves to enrich interpretation.

Further, the progress interface 1500 may be mirrored on wearable devices such as smartwatches or AR glasses, allowing users to discreetly track their ongoing wellness metrics in real-time or receive haptic feedback when a notable trend shift is detected.

In clinic-based settings, the progress log interface may be accessible to authorized personnel across multiple patients, enabling practitioners to conduct cross-comparative analysis, identify therapy response trends across populations, and refine broader therapeutic protocols accordingly.

Taken collectively, the visual constructs and interactive modules depicted in FIG. 15 enable a holistic session logging and feedback framework that supports intelligent therapy evolution, user accountability, and clinical traceability in neurostimulation-based intervention platforms.

### Adaptive Multi-Site Architecture

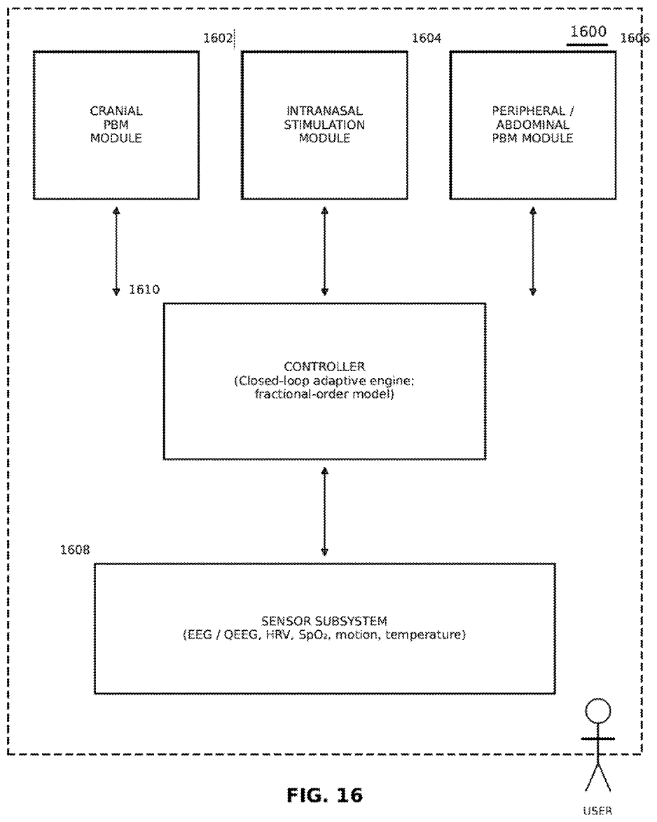

Referring now to FIG. 16, a high-level system architecture 1600 of an adaptive multi-site neurostimulation system is illustrated, in accordance with various embodiments of the present invention. The architecture 1600 may include a cranial photobiomodulation (PBM) module 1602 (which, in some embodiments, includes the head-mounted apparatus 100, 200, 300 of any of the foregoing figures and its emitter portals 102/302), an intranasal stimulation module 1604, and one or more peripheral stimulation modules 1606 (including, in some embodiments, an abdominal PBM applicator). A sensor subsystem 1608 acquires physiological signals from the user, including EEG/QEEG, HRV, and SpO082 signals. A controller 1610 (which may be the controller 1004 of FIG. 10 or a controller integrated with the apparatus 100, 200, 300, or any combination thereof) executes an adaptive control engine that determines and iteratively updates stimulation parameters delivered to one or more of the modules 1602, 1604, 1606. The architecture 1600 is configured to deliver coordinated, multi-site stimulation in which two or more of the modules 1602, 1604, 1606 are operated concurrently or in an alternating schedule based on signals from the sensor subsystem 1608.

In some embodiments, the cranial PBM module 1602 includes one or more energy emitter portals 102 disposed within a wearable housing 100A as described above with respect to FIG. 1, and may emit light in the red and/or near-infrared range (for example, approximately 600013700 nm, approximately 810 nm, approximately 850 nm, approximately 980 nm, or approximately 1070 nm). The cranial PBM module 1602 may include independently addressable portals targeting one or more of the prefrontal, parietal, temporal, occipital, and cerebellar regions.

In some embodiments, the intranasal stimulation module 1604 includes an insertable probe configured to deliver light energy into a nasal cavity of the user. The probe may be configured to direct light energy toward the olfactory mucosa, the cribriform plate, the sphenoidal recess, the ethmoid region, or combinations thereof, thereby providing a relatively short and unobstructed optical path to deep cerebral structures via the cribriform plate and adjacent foramina.

In some embodiments, the one or more peripheral stimulation modules 1606 comprise an abdominal PBM applicator configured to be placed on or proximate to the user019s abdomen, and may emit light in the red and/or near-infrared range to influence gut perfusion, the abdominal microbiome, and SCFA production (including butyrate). Alternatively, or additionally, peripheral stimulation modules 1606 may include cervical (vagus-region) emitters, chest emitters, limb emitters, and electromagnetic emitters.

The controller 1610 may communicate with each of the modules 1602, 1604, 1606 and the sensor subsystem 1608 over a wired bus, a wireless link, or both. The controller 1610 may comprise a microcontroller, system-on-chip (SoC), digital signal processor (DSP), or field-programmable gate array (FPGA), and may run firmware implementing the closed-loop adaptive engine and the fractional-order dynamic model described below.

### Closed-Loop Adaptive Control Subsystem

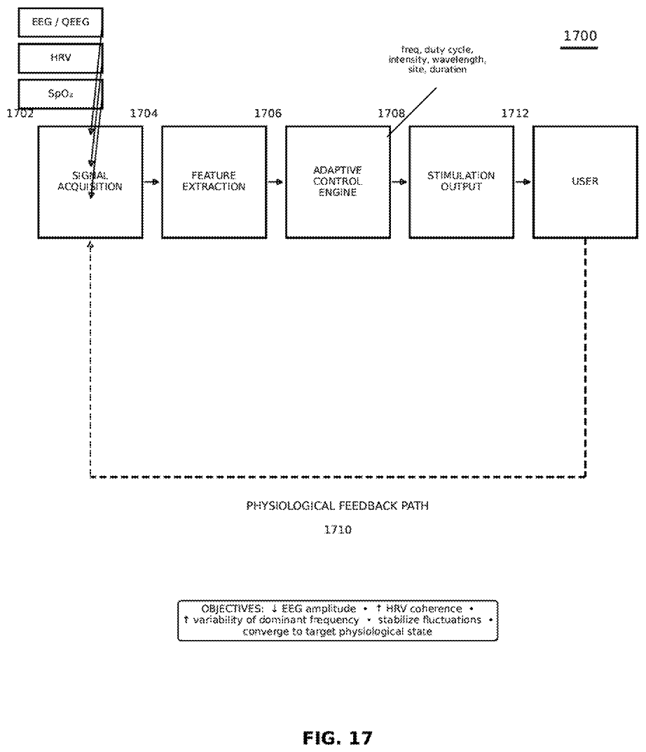

Referring now to FIG. 17, a closed-loop control diagram 1700 is illustrated for the adaptive neurostimulation system, in accordance with various embodiments of the present invention. The closed-loop control diagram 1700 may comprise a signal acquisition block 1702, a feature extraction block 1704, an adaptive control engine 1706, a stimulation output block 1708, and a feedback path 1710 from the user 1712 back to the signal acquisition block 1702.

The signal acquisition block 1702 may acquire one or more of EEG signals (including QEEG features such as power spectral density across delta, theta, alpha, beta, and gamma bands, dominant frequency, amplitude, variability, and inter-channel coherence), HRV signals (including time-domain features such as SDNN and RMSSD, frequency-domain features such as LF and HF power, and non-linear features such as sample entropy), SpO082 signals (including baseline and dynamic response to stimulation), galvanic skin response, respiration, temperature, and motion. EEG acquisition may be performed at sampling rates of at least about 250 Hz, and in some embodiments at sampling rates of about 500 Hz or about 1000 Hz, sufficient to extract dominant frequency, amplitude, and variability metrics.

The feature extraction block 1704 may compute, in real time, one or more features including a dominant EEG frequency, an EEG amplitude, an EEG variability metric, an EEG coherence metric across two or more channels, an HRV metric (for example, RMSSD or LF/HF ratio), and an SpO082 response feature.

The adaptive control engine 1706 may receive the extracted features and may determine one or more stimulation parameters, including frequency, duty cycle, intensity, waveform, wavelength selection (for example, selecting between approximately 810 nm and approximately 1070 nm), site selection (for example, selecting between cranial, intranasal, and peripheral modules), and duration. The adaptive control engine 1706 may issue commands to the stimulation output block 1708, which drives the corresponding emitters in the modules 1602, 1604, 1606.

The feedback path 1710 closes the loop. Stimulation alters the user019s physiological state, which alters the acquired signals, which alters the extracted features, which causes the adaptive control engine 1706 to update the stimulation parameters. The stimulation parameters are not selected solely from a predefined program, are not fixed prior to the session, and may vary continuously during the session. In some embodiments, the stimulation parameters are updated at least once per second, and in some embodiments substantially continuously.

Examples of adaptive control objectives include reducing EEG amplitude (for example, reducing excessive beta amplitude associated with arousal), increasing variability of a dominant frequency, improving HRV coherence, stabilizing physiological fluctuations, and converging toward a target physiological state defined by one or more of the foregoing features.

### Fractional-Order Dynamic Model

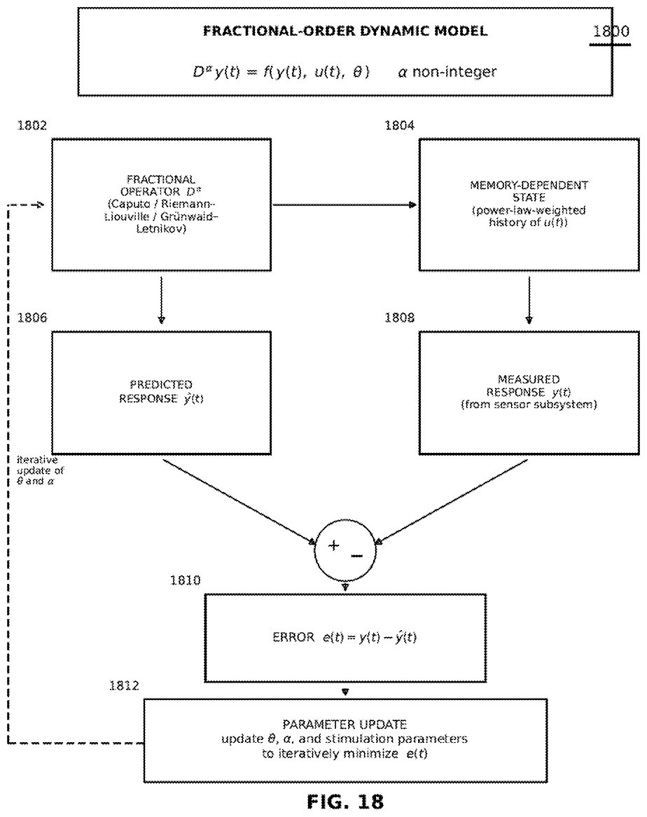

Referring now to FIG. 18, a schematic 1800 of a fractional-order dynamic model employed by the controller is illustrated, in accordance with various embodiments of the present invention. The schematic 1800 may include a fractional-order operator block 1802, a memory-dependent state block 1804, a predicted-response block 1806, a measured-response block 1808 supplied from the sensor subsystem, an error block 1810, and a parameter update block 1812.

In some embodiments, the fractional-order dynamic model is defined by one or more non-integer order differential equations of the general form D\hat{ }3b1 y(t) =f(y(t), u(t), 3b8), where D\hat{ }3b1 denotes a fractional differential operator of order 3b1, where 3b1 is a non-integer real number (for example, between 0 and 1, or between 0 and 2), y(t) denotes a physiological state vector (including, for example, dominant EEG frequency, HRV metrics, and/or SpO082), u(t) denotes the stimulation input (including, for example, frequency, duty cycle, intensity, and wavelength), and 3b8 denotes one or more model parameters. The fractional operator may be defined using Caputo, Riemann013Liouville, Gr0fcnwald013Letnikov, or other suitable formulations known in the art of fractional calculus. The non-integer order 3b1 captures memory-dependent behavior of the physiological system, in which present response depends on a weighted history of prior inputs, with weights decaying as a power-law function of elapsed time.

The predicted-response block 1806 computes a predicted physiological response based on the model and a proposed stimulation input. The error block 1810 generates an error signal based on a comparison of the predicted response to the measured response. The parameter update block 1812 updates one or more of the stimulation parameters and the model parameters (3b8, including the fractional order 3b1) to reduce the error signal over successive iterations, thereby iteratively minimizing prediction error across the session and across successive sessions.

In some embodiments, the fractional-order model is initialized at the start of each session using parameters stored from prior sessions for the same user, and is continuously updated during the session based on observed data. In some embodiments, the controller stores historical physiological data, stimulation parameters, and outcome metrics in a memory, and uses the stored data both to initialize subsequent sessions and to improve parameter selection across successive sessions.

### Intranasal Stimulation Module

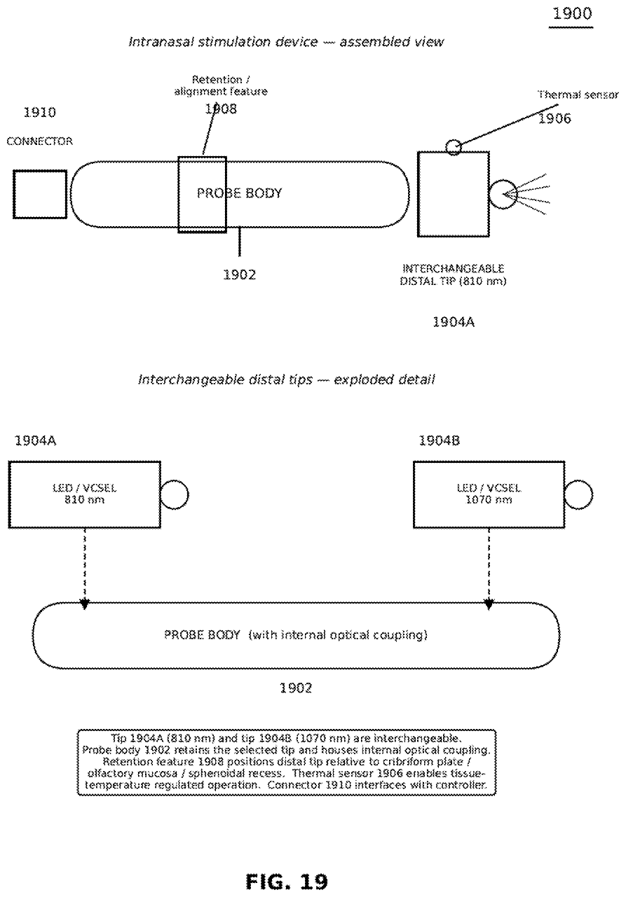

Referring now to FIG. 19, an intranasal stimulation device 1900 is illustrated, in accordance with various embodiments of the present invention. The intranasal stimulation device 1900 may comprise a probe body 1902, one or more interchangeable distal tips 1904A, 1904B configured to be releasably engaged with the probe body 1902, a thermal sensor 1906, a retention or alignment feature 1908, and a connector 1910 configured to interface with a controller. The intranasal stimulation device 1900 may be operatively coupled to the controller 1610 of FIG. 16, the controller 1004 of FIG. 10, or any other controller described herein, by a wired connection via the connector 1910 or by a wireless link.

In some embodiments, the one or more interchangeable distal tips 1904A, 1904B are each configured to emit light at a respective wavelength selected from approximately 810 nm and approximately 1070 nm. Each distal tip 1904A, 1904B may include one or more light-emitting elements (for example, one or more laser diodes, light-emitting diodes (LEDs), or vertical-cavity surface-emitting lasers (VCSELs)) and one or more diffusers or optical waveguides configured to distribute the emitted light over a target region of the nasal cavity. The interchangeable distal tips 1904A, 1904B may be exchanged by a user or clinician to select wavelength, beam profile, or anatomical target without replacement of the entire intranasal stimulation device 1900.

In some embodiments, the intranasal stimulation device 1900 is configured to deliver an output optical power of at least about 50 mW, and in particular embodiments of approximately 80 mW, at the wavelength(s) selected from approximately 810 nm and approximately 1070 nm. In some embodiments, the controller is configured to operate the intranasal stimulation device 1900 with a pulsed waveform at frequencies including approximately 10 Hz, approximately 40 Hz, approximately 48 Hz, or approximately 432 Hz, with a duty cycle between approximately 30% and approximately 90%, and for a session duration between approximately 15 minutes and approximately 60 minutes.

The thermal sensor 1906 may be disposed on or proximate to the interchangeable distal tip 1904A, 1904B and may be configured to measure tip temperature, tissue temperature, or both. The controller may receive thermal-sensor data and may reduce optical output power, modify duty cycle, or terminate stimulation in response to a thermal threshold being met or exceeded, thereby regulating safe operation within nasal tissues.

The retention or alignment feature 1908 may comprise an elastomeric collar, an adjustable bridge clip, a flexible support arm, an inflatable cuff, or a similar structure configured to position the probe body 1902 such that the distal tip 1904A, 1904B is positioned with respect to a target nasal anatomy, including, by way of example and not limitation, the cribriform plate, the olfactory mucosa, the sphenoidal recess, or the ethmoid region.

### Parameter Optimization Layer

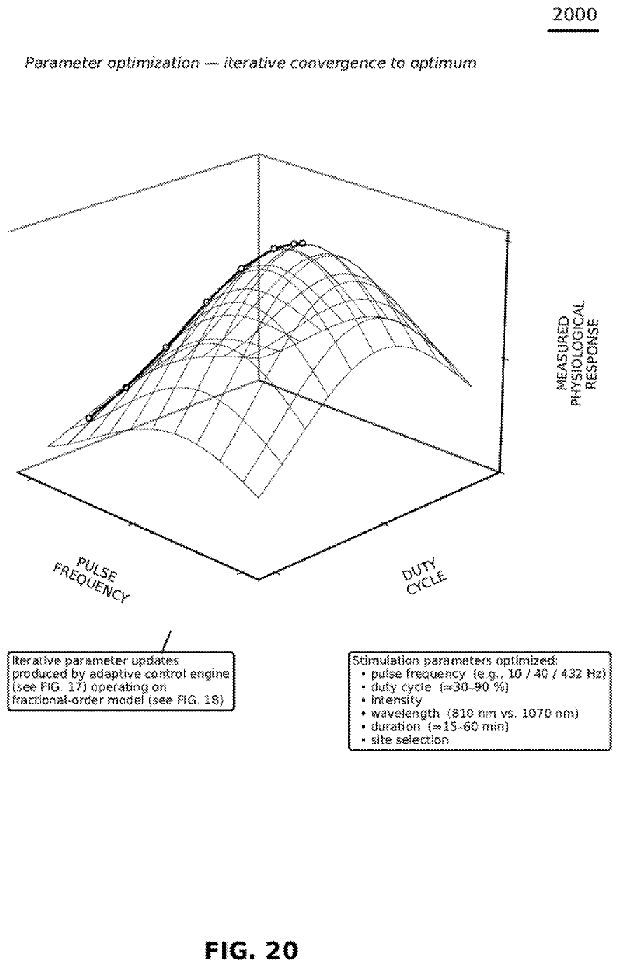

Referring now to FIG. 20, a parameter optimization layer 2000 is illustrated in accordance with various embodiments of the present invention. The parameter optimization layer 2000 illustrates iterative optimization of one or more stimulation parameters, including frequency, duty cycle, intensity, wavelength, and duration, by reference to one or more measured physiological response surfaces. Each axis represents a stimulation parameter, and a surface or set of trajectories represents the measured physiological response (for example, change in HRV coherence, change in EEG dominant frequency variability, or convergence toward a target SpO082 response). The controller selects parameter values that move the measured response toward an optimization objective (for example, maximization of HRV coherence or minimization of dominant-frequency variance) using gradient-based, model-predictive, or reinforcement-learning techniques operating on the fractional-order model of FIG. 18.

In some embodiments, the controller maintains a frequency response map linking PBM pulse frequency to one or more physiological outputs (for example, HRV response, SpO082 response, and EEG-band shifts). The frequency response map may be updated during the session and across sessions, providing a functional mapping layer between photonic frequency and biological response. The frequency response map may include data points at approximately 10 Hz, approximately 40 Hz, approximately 48 Hz, approximately 432 Hz, and additional non-linear frequencies.

### Multi-Modal Integration and Dual-Input Energetic Model

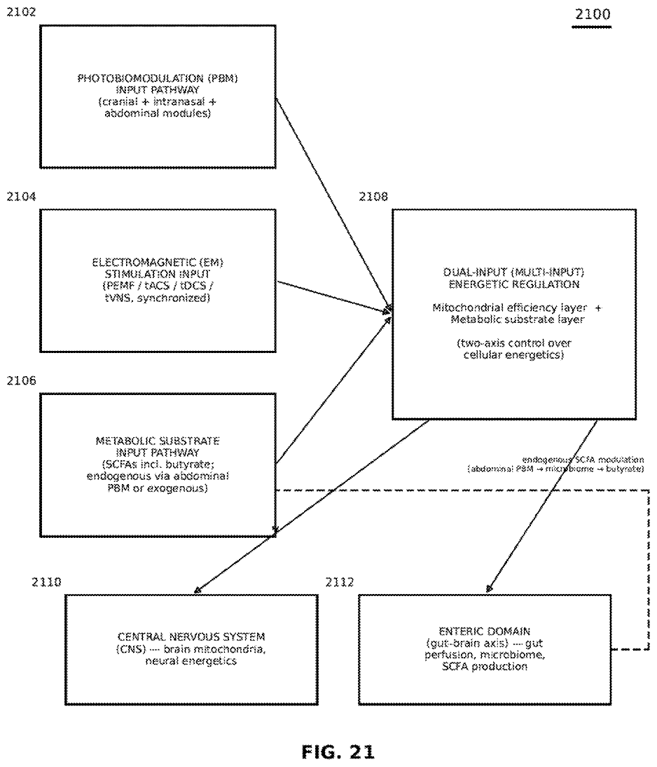

Referring now to FIG. 21, a multi-modal integration architecture 2100 is illustrated, in accordance with various embodiments of the present invention. The architecture 2100 includes a photobiomodulation input pathway 2102, an electromagnetic stimulation input pathway 2104, and a metabolic-substrate input pathway 2106 that converge on a dual-input (or multi-input) energetic regulation block 2108 representing mitochondrial and neural energetics across central nervous system (CNS) and enteric domains.

The photobiomodulation input pathway 2102 represents stimulation delivered by one or more of the cranial PBM module 1602, the intranasal stimulation module 1604, and one or more peripheral PBM modules 1606, and serves as a mitochondrial efficiency input by enhancing cytochrome c oxidase activity, mitochondrial membrane potential, and adenosine triphosphate (ATP) production.

The electromagnetic stimulation input pathway 2104 represents adjunctive electromagnetic stimulation, which may include pulsed electromagnetic field (PEMF) stimulation, transcranial alternating current stimulation (tACS), transcranial direct current stimulation (tDCS), or transcutaneous vagus nerve stimulation (tVNS), synchronized in time and/or in frequency with the photobiomodulation input pathway 2102 by the controller.

The metabolic-substrate input pathway 2106 represents the availability of one or more metabolic substrates, including short-chain fatty acids (SCFAs) such as butyrate, acetate, and propionate, ketone bodies, or other endogenous metabolic substrates. The metabolic-substrate input pathway 2106 may be modulated endogenously by abdominal PBM (which influences gut perfusion, microbiome composition, and SCFA production), exogenously by oral or enteral administration of a metabolic substrate (for example, a butyrate or SCFA precursor), or by combinations thereof.

The dual-input (or multi-input) energetic regulation block 2108 represents a system in which mitochondrial and neural energetics in CNS and enteric tissues are regulated by the concurrent operation of (i) a mitochondrial efficiency layer provided by photobiomodulation and (ii) a metabolic substrate layer provided by SCFAs (such as butyrate), thereby providing a two-axis control system over cellular energetics that is distinct from prior PBM-only and prior stimulation-only systems. The architecture 2100 thus engages the gut013brain axis as part of the closed-loop regulatory system, in that abdominal PBM may alter SCFA availability, SCFA availability may alter CNS metabolic readiness, and CNS metabolic readiness may, in turn, alter the measured physiological response upon which the closed-loop adaptive engine of FIG. 17 acts.

### Learning Across Sessions

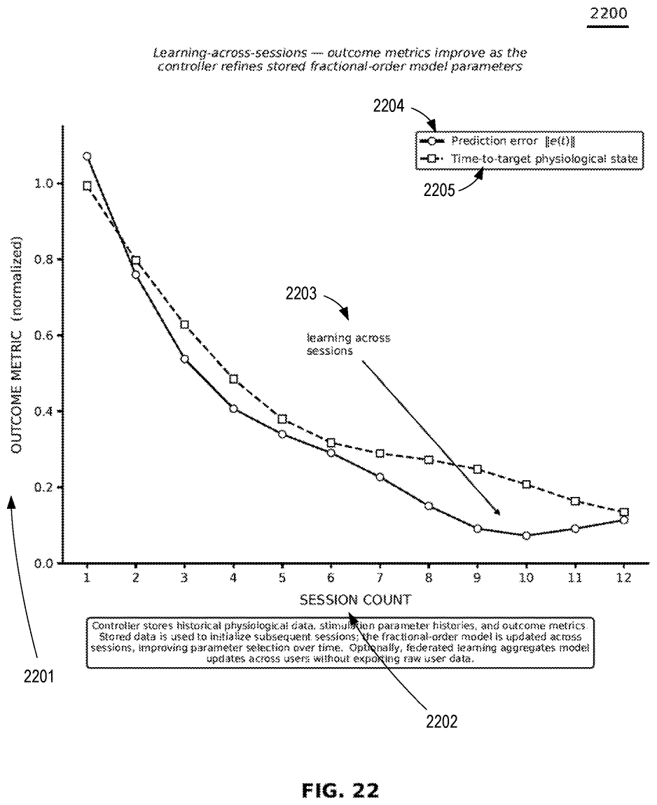

Referring now to FIG. 22, a learning curve 2200 is illustrated, in accordance with various embodiments of the present invention. The learning curve 2200 plots one or more outcome metrics 2201, such as model prediction error 2204, time-to-target physiological state 2205, parameter-update magnitude, or a normalized therapeutic response score, against a session count 2202 (for example, sessions 1 through N) for a given user. As the controller accumulates historical physiological data and updates the fractional-order model of FIG. 18, the outcome metric improves (for example, the prediction error decreases, time-to-target decreases, or therapeutic response score increases), illustrating learning across sessions 2203.

In some embodiments, the controller is configured to (a) store historical physiological data, including time-aligned EEG/QEEG, HRV, and SpO082 data, stimulation parameter histories, and outcome metrics, in a memory; (b) use the stored historical data to initialize subsequent sessions; and (c) improve parameter selection across sessions, including by updating the fractional-order model parameters 3b8 and the fractional order 3b1 in response to inter-session data.

In some embodiments, learning across sessions is performed using federated learning across multiple users, wherein model updates are aggregated across users without exporting raw user data, thereby preserving user privacy while improving population-level model parameters.

Differentiation From Prior Photobiomodulation and Neurostimulation Systems

The present invention differs from prior photobiomodulation systems, including but not limited to systems marketed by Vielight Inc., in that the present system: (a) is adaptive rather than fixed, in that stimulation parameters are iteratively adjusted during the session based on measured physiological response; (b) is multi-site, including coordinated cranial, intranasal, and peripheral (including abdominal) stimulation; (c) is multi-input, integrating EEG/QEEG, HRV, and SpO082 in real time; (d) integrates metabolic substrate optimization (including SCFAs such as butyrate) as a co-regulator of mitochondrial function; and (e) employs a fractional-order, memory-dependent adaptive control engine. The present invention is, accordingly, a platform for adaptive energetic regulation of the nervous system rather than a fixed-protocol stimulation device.

The stimulation parameters of the present invention are not selected solely from a predefined program, are not fixed prior to the session, and are driven by measured physiological response during the session. The parameters may vary continuously during the session.

### Illustrative Example 013 Adaptive Multi-Site Session

In an illustrative example of operation, a user dons the head-mounted apparatus 100, 200, or 300 and inserts the intranasal stimulation device 1900 into a nostril. An abdominal PBM applicator is positioned on the user019s abdomen. The controller begins a session by retrieving the user019s stored fractional-order model from memory and initializing the stimulation parameters. The sensor subsystem 1608 begins acquiring EEG, HRV, and SpO082 signals. The feature extraction block 1704 extracts dominant alpha-band frequency, RMSSD, and SpO082 dynamic response. The adaptive control engine 1706 selects a starting parameter set comprising 10 Hz pulsed PBM at the prefrontal portals of the cranial module 1602, 40 Hz pulsed PBM at the intranasal module 1604 at approximately 810 nm and approximately 80 mW, and continuous-wave abdominal PBM at the peripheral module 1606. During the session, the user019's HRV coherence decreases briefly; the adaptive control engine 1706 detects the change, the fractional-order model predicts that a transition to 432 Hz at the prefrontal portals will improve coherence, and the controller updates parameters accordingly. encephalographicAfter 30 minutes, the controller terminates the session and updates the stored model based on the session019s observed data. Outcome metrics, stimulation parameter histories, and time-aligned physiological data are written to memory and optionally transmitted to a clinician dashboard or research server.

In accordance with various embodiments of the present invention, a neurostimulation apparatus may incorporate a plurality of optional and modular features, wherein one or more of these features may be selectively utilized based on the therapy objective, user preference, usage environment, or system configuration. It is expressly contemplated that the apparatus is not limited to a configuration requiring simultaneous activation or use of all disclosed components and systems. Rather, the invention enables flexible deployment and adaptive use, where distinct hardware and software subsystems may be engaged independently or in logical combinations to support varying therapeutic goals and user needs.

In some embodiments, the neurostimulation apparatus may be used in its most basic operational configuration comprising only the wearable stimulation device with embedded energy emitter portals and internal memory to store predefined stimulation routines. In such embodiments, the apparatus may function without real-time sensor feedback, user interaction via interface devices, or external communication. A user may initiate therapy by pressing a physical button on the apparatus, thereby activating a default stimulation profile stored internally.

In another embodiment, the neurostimulation apparatus may be paired with a user device, such as a smartphone or tablet, operatively connected via a wired or wireless interface. In this case, the user device may be employed to customize therapy parameters, view historical performance logs, or receive real-time feedback. The user device may display graphical representations of current therapy metrics, provide options for selecting predefined routines (e.g., for sleep, attention, relaxation), or receive remote recommendations from medical personnel. This feature may be used in combination with or independently of other components such as sensing probes or biometric inputs.

In some embodiments, biometric systems may be engaged to personalize and secure access to the apparatus. For example, the apparatus may be configured to recognize a user's voice, fingerprint, or facial image via built-in or peripheral sensors. A microphone in the apparatus may be configured to perform voiceprint authentication or detect user tone to infer emotional state, which can be used to inform therapy selection. Alternatively, a fingerprint scanner located on a paired user device may authenticate access to user-specific settings or initiate profile-based stimulation routines. These biometric features may be used exclusively for user authentication or may be further leveraged for adaptive emotional state analysis.

The apparatus may also be optionally used with a modular goggle accessory designed to deliver stimulation to ocular and periorbital regions. In some embodiments, the goggle may include a plurality of light emitters surrounding the visual field, configured to emit light without projecting directly into the eyes. The goggle may be removably attachable to the helmet-based apparatus via electrical connectors or wireless control. In an alternate embodiment, the goggle may be operated independently of the helmet for vision-specific therapies, such as photobiomodulation, circadian rhythm regulation, or ocular strain reduction.

In another embodiment, one or more peripheral sensing probes may be connected to the apparatus or user device via detachable leads. These probes may be positioned on the chest, arms, legs, or other regions of the body, and may comprise sensors for measuring heart rate variability (HRV), galvanic skin response, skin temperature, blood oxygen saturation, or motion. In certain configurations, only one sensor probe may be engaged—such as a chest sensor for HRV monitoring—while in others, multiple probes may be concurrently deployed. The apparatus may analyze data from these sensors to determine autonomic or metabolic states and update therapy parameters accordingly.

In some implementations, the apparatus may comprise a microphone and/or speaker to enable audio interaction. The microphone may be configured to receive verbal commands, log voice-based feedback, or perform vocal tone analysis to estimate stress, fatigue, or alertness. The speaker may be used to deliver guided meditation, verbal cues, or therapy sounds (e.g., binaural beats, nature ambience). It is contemplated that the speaker and microphone functions may be optionally enabled or disabled based on user preference or regulatory requirements.

It is also contemplated that the various emitter portals and therapy stimulation routines may be engaged regardless of whether sensing probes, biometric authentication, or a user device is present. For example, in environments where wireless communication is prohibited or discouraged—such as within medical or aerospace facilities—the apparatus may operate in a local mode, storing and executing therapy plans without requiring live sensor input or internet connectivity.

In another example, the apparatus may be used by a clinic where different patients are administered therapy in succession. In this case, user authentication via biometric or manual profile switching may be utilized to retrieve personalized settings, while other features like emotion detection or speech input may remain inactive to maintain focus on preconfigured treatment routines.

Moreover, the AI engine referenced in multiple embodiments may be implemented in the apparatus itself, the user device, or a cloud server. This AI engine may receive input from any of the operational components—sensors, microphones, goggle emitters, user interaction logs—and adapt therapy patterns dynamically. However, use of the AI engine is not mandatory for basic operation. In some embodiments, the apparatus may function using static rule-based logic or factory-programmed profiles, while the AI engine is reserved for users requiring advanced personalization.

Thus, the invention provides a flexible system architecture wherein features such as: (i) connected user devices; (ii) biometric authentication and emotional analysis; (iii) goggle-based emitter arrays; (iv) sensing probes; (v) microphone and speaker input/output; and (vi) embedded or remote AI intelligence, may be selectively used in isolation or in combination based on the user's medical profile, therapy objective, technical environment, or regulatory constraints. This modular, interoperable framework expands the usability of the apparatus across different domains, including personal wellness, clinical therapy, cognitive rehabilitation, remote monitoring, and neuro-augmentation research.

Accordingly, the invention encompasses all such configurations where any one or more features are enabled or disabled dynamically or by design, without departing from the scope and utility of the neurostimulation system described herein.

### CONCLUSION

A number of embodiments of the present disclosure have been described. While this specification contains many specific implementation details, they should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings, and the detailed description thereto, are not intended to limit the present disclosure to the form disclosed, but to the contrary, the present disclosure is to cover all modifications, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” be used in a permissive sense (i.e., meaning having the potential to), rather than the mandatory sense (i.e., meaning must). Similarly, the words “include,” “including,” and “includes” mean including but not limited to. To facilitate understanding, like reference numerals have been used, where possible, to designate like elements common to the figures.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising,” “including,” and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A neurostimulation system comprising:
a cranial photobiomodulation module configured to deliver photobiomodulation energy to a cranial region of a user;
an intranasal stimulation module configured to deliver light energy into a nasal cavity of the user;
an abdominal photobiomodulation module configured to deliver photobiomodulation energy to an abdominal region of the user to modulate a metabolic-substrate input pathway associated with short-chain fatty acid availability;
a sensor subsystem configured to acquire, in real time during a stimulation session, physiological signals comprising an electroencephalographic signal, a heart rate variability signal, and a peripheral oxygen saturation signal; and
a controller operatively coupled to the cranial photobiomodulation module, the intranasal stimulation module, the abdominal photobiomodulation module, and the sensor subsystem, the controller comprising a processor and a memory storing instructions that, when executed by the processor, cause the controller to:
extract, from the physiological signals, a plurality of physiological features comprising an electroencephalographic feature, a heart rate variability feature, and a peripheral oxygen saturation response feature;
maintain a fractional-order dynamic model representing memory-dependent physiological response of the user to stimulation delivered by the cranial photobiomodulation module, the intranasal stimulation module, and the abdominal photobiomodulation module;
generate, using the fractional-order dynamic model, a predicted physiological response of the user to a candidate stimulation input that includes respective stimulation parameters for the cranial photobiomodulation module, the intranasal stimulation module, and the abdominal photobiomodulation module;
compare the predicted physiological response with a measured physiological response derived from the plurality of physiological features to generate an error signal;
select updated stimulation parameters for coordinated operation of the cranial photobiomodulation module, the intranasal stimulation module, and the abdominal photobiomodulation module based on the error signal and the metabolic-substrate input pathway; and
iteratively update the updated stimulation parameters during the stimulation session to converge the measured physiological response toward a target physiological state.

2. The neurostimulation system of claim 1, wherein the fractional-order dynamic model includes a non-integer order differential equation having a fractional differential operator of order α, where α is a non-integer real number greater than zero and less than or equal to two.

3. The neurostimulation system of claim 2, wherein the controller is configured to update α during the stimulation session based on the measured physiological response.

4. The neurostimulation system of claim 1, wherein the fractional-order dynamic model is formulated using a Caputo derivative, a Riemann-Liouville derivative, or a Grunwald-Letnikov derivative.

5. The neurostimulation system of claim 1, wherein the controller is configured to initialize the fractional-order dynamic model at commencement of the stimulation session using model parameters stored in the memory from a prior stimulation session of the user.

6. The neurostimulation system of claim 1, wherein the controller is configured to store time-aligned physiological data, stimulation parameter histories, and outcome metrics in the memory after the stimulation session.

7. The neurostimulation system of claim 1, wherein the controller is configured to reduce a prediction error of the fractional-order dynamic model across successive stimulation sessions of the user using historical physiological data stored in the memory.

8. The neurostimulation system of claim 1, wherein the controller is configured to update one or more model parameters of the fractional-order dynamic model using federated learning across a plurality of users while preserving raw physiological data of the user within the neurostimulation system.

9. The neurostimulation system of claim 1, wherein the intranasal stimulation module includes an insertable probe configured to emit near-infrared light at an emission wavelength selected by the controller between about 810 nm and about 1070 nm during the stimulation session.

10. The neurostimulation system of claim 9, wherein the insertable probe includes an interchangeable distal tip configured to direct the near-infrared light toward an olfactory mucosa, a cribriform plate, a sphenoidal recess, or an ethmoid region of the user.

11. The neurostimulation system of claim 9, wherein the insertable probe includes a thermal sensor configured to provide thermal-sensor data to the controller for reducing an optical output power, modifying a duty cycle, or terminating stimulation in response to a thermal threshold.

12. The neurostimulation system of claim 9, wherein the insertable probe includes a retention or alignment feature configured to position the insertable probe relative to target nasal anatomy of the user.

13. The neurostimulation system of claim 1, wherein the cranial photobiomodulation module includes a wearable housing including a plurality of independently controllable emitter portals positioned to deliver light energy to a frontal region, a parietal region, an occipital region, or a cerebellar region of the user.

14. The neurostimulation system of claim 13, wherein at least one independently controllable emitter portal includes an emitter-sensor pair configured to deliver stimulation energy to a localized cranial region and detect a physiological signal from the localized cranial region.

15. The neurostimulation system of claim 1, wherein the electroencephalographic feature includes a dominant frequency, an amplitude, a variability metric, an inter-channel coherence metric, or a power spectral density across a delta band, a theta band, an alpha band, a beta band, or a gamma band.

16. The neurostimulation system of claim 1, wherein the heart rate variability feature includes an RMSSD value, an SDNN value, an LF power value, an HF power value, an LF/HF ratio, or a sample entropy value.

17. The neurostimulation system of claim 1, wherein the controller is configured to select a pulse frequency, a duty cycle, an intensity, a waveform, a wavelength, a site selection, or a session duration as one of the updated stimulation parameters.

18. The neurostimulation system of claim 1, wherein the controller is configured to maintain a frequency response map linking a photobiomodulation pulse frequency to the measured physiological response.

19. The neurostimulation system of claim 18, wherein the frequency response map includes data points corresponding to about 10 Hz, about 40 Hz, about 48 Hz, or about 432 Hz.

20. The neurostimulation system of claim 1, wherein the controller is configured to synchronize an electromagnetic stimulation input pathway with a photobiomodulation input pathway of the cranial photobiomodulation module, the intranasal stimulation module, or the abdominal photobiomodulation module.
